(19) United States
(12) Patent Application Publication
(10) Pub. No.: US 2013/0084579 A1
(43) Pub. Date: Apr. 4, 2013

(54) DRUG SUSCEPTIBILITY USING RARE CELL DETECTION SYSTEM

(71) Applicant: Battelle Memorial Institute, Columbus, OH (US)

(72) Inventors: Herbert S. Bresler, Bexley, OH (US); Kevin M. Taylor, Columbus, OH (US); John S. Laudo, Hilliard, OH (US); Thomas D. Haubert, Columbus, OH (US)

(73) Assignee: Battelle Memorial Institute, Columbus, OH (US)

(21) Appl. No.: 13/622,740

(22) Filed: Sep. 19, 2012

Related U.S. Application Data
(63) Continuation-in-part of application No. 13/225,074, filed on Sep. 2, 2011.
Application No. 13/225,074 is a continuation of application No. 13/052,151, filed on Mar. 21, 2011, now Pat. No. 8,012,742.
Application No. 13/052,151 is a continuation of application No. 12/029,274, filed on Feb. 11, 2008, now Pat. No. 7,915,029.
Application No. 12/029,274 is a continuation of application No. 11/370,635, filed on Mar. 7, 2006, now Pat. No. 7,329,534.
Application No. 11/370,635 is a division of application No. 10/263,975, filed on Oct. 3, 2002, now Pat. No. 7,074,577.
Application No. 10/263,975 is a continuation-in-part of application No. 12/498,533, filed on Jul. 7, 2009.
Application No. 12/498,533 is a division of application No. 11/519,533, filed on Sep. 12, 2006, now Pat. No. 7,560,277.
Application No. 11/519,533 is a division of application No. 11/261,105, filed on Oct. 27, 2005, now Pat. No. 7,397,601.
Application No. 11/261,105 is a continuation-in-part of application No. 13/371,761, filed on Feb. 13, 2012.
Application No. 13/371,761 is a continuation-in-part of application No. 13/052,172, filed on Mar. 21, 2011, now Pat. No. 8,114,680.
Application No. 13/052,172 is a continuation of application No. 12/614,602, filed on Nov. 9, 2009, now Pat. No. 7,919,049.
Application No. 12/614,602 is a continuation of application No. 12/029,289, filed on Feb. 11, 2008, now Pat. No. 7,629,176.
Application No. 12/029,289 is a continuation of application No. 11/609,186, filed on Dec. 11, 2006, now Pat. No. 7,358,095.
Application No. 11/609,186 is a continuation of application No. 10/263,974, filed on Oct. 3, 2002, now Pat. No. 7,220,593.
Application No. 10/263,974 is a continuation-in-part of application No. PCT/US2011/030414, filed on Mar. 30, 2011.
(60) Provisional application No. 61/536,303, filed on Sep. 19, 2011.
(60) Provisional application No. 60/631,025, filed on Nov. 24, 2004.
(60) Provisional application No. 60/631,026, filed on Nov. 24, 2004.
(60) Provisional application No. 60/631,027, filed on Nov. 24, 2004.

(51) Int. Cl. G01N 33/94

(52) U.S. Cl. CPC G01N 33/94
USPC 435/7.1

(57) ABSTRACT

Methods for determining the efficacy of a given drug for a specific patient with cancer in vitro prior to, or after, the initiation of treatment of the patient are disclosed. Blood from the cancer patient is separated into an assay test tube and a control test tube. The blood in the assay test tube is exposed to a cancer drug. The two test tubes are then visually examined and compared to determine the effect of the cancer drug on cancer cells, other rare cells in the blood, or on normal constituents of the blood of a cancer patient.

### CROSS-REFERENCE TO RELATED APPLICATIONS

This application claims priority to U.S. Provisional Patent Application Ser. No. 61/536,303, filed on Sep. 19, 2011. This application is also a continuation-in-part of U.S. patent application Ser. No. 13/225,074, filed Sep. 2, 2011, which is a continuation of U.S. Pat. No. 8,012,742, filed Mar. 21, 2011, which was a continuation of U.S. Pat. No. 7,915,029, filed Feb. 11, 2008, which was a continuation of U.S. Pat. No. 7,329,534, filed Mar. 7, 2006, which was a divisional of U.S. Pat. No. 7,074,577, filed Oct. 3, 2002. This application is also a continuation-in-part of U.S. patent application Ser. No. 12/498,533, filed Jul. 7, 2009, which is a divisional of U.S. Pat. No. 7,560,277, filed Sep. 12, 2006, which was a divisional application of U.S. Pat. No. 7,397,601, filed Oct. 27, 2005, which claimed priority to three different provisional applications: U.S. Provisional Patent Application Ser. No. 60/631,025, filed Nov. 24, 2004; U.S. Provisional Patent Application Ser. No. 60/631,026, filed Nov. 24, 2004; and U.S. Provisional Patent Application Ser. No. 60/631,027, filed Nov. 24, 2004. This application is also a continuation-in-part of U.S. patent application Ser. No. 13/371,761, filed Feb. 13, 2012, which is a continuation of U.S. Pat. No. 8,114,680, filed on Mar. 21, 2011, which was a continuation of U.S. Pat. No. 7,919,049, filed Nov. 9, 2009, which was a continuation of U.S. Pat. No. 7,629,176, filed Feb. 11, 2008, which was a continuation of U.S. Pat. No. 7,358,095, filed Dec. 11, 2006, which was a continuation of U.S. Pat. No. 7,220,593, filed Oct. 3, 2002. This application is also a continuation-in-part of PCT Application No. PCT/US2011/030414, filed Mar. 30, 2011, which claimed priority to U.S. Provisional Patent Application Ser. No. 61/318,903, filed on Mar. 30, 2010, and to U.S. Provisional Patent Application Ser. No. 61/372,889, filed on Aug. 12, 2010. This application is also a continuation-in-part of PCT Application No. PCT/US2011/030417, filed Mar. 30, 2011, which claimed priority to U.S. Provisional Patent Application Ser. No. 61/318,912, filed on Mar. 30, 2010, and to U.S. Provisional Patent Application Ser. No. 61/372,900, filed on Aug. 12, 2010. This application is also a continuation-in-part of PCT Application No. PCT/US2011/030420, filed Mar. 30, 2011, which claimed priority to U.S. Provisional Patent Application Ser. No. 61/318,929, filed on Mar. 30, 2010, and to U.S. Provisional Patent Application Ser. No. 61/372,905, filed on Aug. 12, 2010. The disclosures of these applications are hereby incorporated by reference in their entirety.

### BACKGROUND

The present disclosure relates to methods for determining the efficacy of a given drug for a specific patient with cancer in vitro prior to the initiation of treatment of the patient or after treatment to determine whether drug resistance has developed. The methods may also be useful in evaluating the toxicity of a given drug, or for quantifying the needed amount of a given drug. Generally, the methods may facilitate personalized medicine, enabling the choice and/or amount of drug to be tailored to the individual patient. The methods may also be useful experimentally as part of the drug development process.

Cancers, or malignant neoplasms, belong to a class of diseases in which a group of cells display uncontrolled growth, invade and destroy adjacent tissues, and metastasize (i.e. spread to other locations in the body). Cancers are one of the leading causes of disease in the world. They attack many different organs in the human body.

Cancers can be treated in many ways. Some drugs can be applied or taken by the cancer patient. Radiation treatment can be used to kill the cancerous tumor cells. Surgery can also be used to remove cancerous tumors, or the organs/body parts infected by such tumors.

In particular, many different types of anti-cancer drugs exist. These types include alkylating agents, antimetabolites, plant alkaloids, inhibitors of various enzymes, and monoclonal antibodies. Many different anti-cancer drugs have been synthesized. These drugs include, for example, vinblastine, vincristine, vinflunine, vindesine, vinorelbine, cabazitaxel, docetaxel, larotaxel, ortataxel, paclitaxel, tesetaxel, ixabepilone, aminopterin, methotrexate, pemetrexed, pralatrexate, raltitrexed, pemetrexed, pentostatin, cladribine, clofarabine, fludarabine, thioguanine, mercaptopurine, fluorouracil, capecitabine, tegafur, carmofur, floxuridine, cytarabine, gemcitabine, azacitidine, decitabine, hydroxycarbamide, camptothecin, topotecan, irinotecan, rubitecan, belotecan, etoposide, teniposide, aclarubicin, daunorubicin, doxorubicin, epirubicin, idarubicin, amrubicin, pirarubicin, valrubicin, zorubicin mitoxantrone, pixantrone, mechlorethamine, ifosfamide, trofosfamide, chlorambucil, melphalan, prednimustine, bendamustine, uramustine, estramustine, carmustine, lomustine, semustine, fotemustine, nimustine, ranimustine, streptozocin, busulfan, mannosulfan, treosulfan, carboquone, thiotepa, triaziquone, triethylenemelamine, carboplatin, cisplatin, nedaplatin, oxaliplatin, triplatin tetranitrate, satraplatin, procarbazine, dacarbazine, temozolomide, altretamine, mitobronitol, actinomycin, bleomycin, mitomycin, plicamycin, aminolevulinic acid/methyl aminolevulinate, efaproxiral, porfimer sodium, talaporfin, temoporfin, verteporfin, tipifamib, alvocidib, seliciclib, bortezomib, anagrelide, tiazofurine, masoprocol, olaparib, vorinostat, romidepsin, atrasentan, bexarotene, testolactone, amsacrine, trabectedin, alitretinoin, tretinoin, arsenic trioxide, asparaginase/pegaspargase, celecoxib, demecolcine, elesclomol, elsamitrucin, etoglucid, lonidamine, lucanthone, mitoguazone, mitotane, oblimersen, omacetaxine mepesuccinate, everolimus, temsirolimus, cetuximab, panitumumab, trastuzumab, catumaxomab, edrecolomab, bevacizumab, ibritumomab, ofatumumab, rituximab, tositumomab, alemtuzumab, gemtuzumab, erlotinib, gefitinib, vandetanib, afatinib, lapatinib, neratinib, axitinib, pazopanib, sunitinib, sorafenib, toceranib, lestaurtinib, axitinib, cediranib, pazopanib, regorafenib, semaxanib, sorafenib, sunitinib, toceranib, vandetanib, dasatinib, imatinib, nilotinib, bosutinib, lestaurtinib, crizotinib, aflibercept, and denileukin diftitox.

Unfortunately, anti-cancer drugs have many adverse side effects. These adverse side effects include immune system depression, infections, fatigue, tendency to bleed easily, nausea, vomiting, diarrhea, constipation, hair loss, damage to certain organs, infertility, pain or paralysis, and impotence. Many of these anti-cancer drugs target biological processes that simply occur in tumor cells at a faster rate than in healthy cells, but healthy cells can be affected as well. Each drug has a different side effect profile, and each drug also works differently between patients. In other words, a drug that works in one patient may be ineffective in another patient.

The conventional way of selecting an anti-cancer drug for use in a particular patient involves prescribing a drug and correcting the prescription based on an observation of the patient's response to the drug. This approach can be time-consuming and cause adverse side effects in the patient. It would be desirable to provide alternative methods or processes for determining what drugs are effective in a particular patient, or put another way which drugs the tumors in a particular patient are susceptible to.

### BRIEF DESCRIPTION

The present disclosure relates to methods and processes for testing drug susceptibility in a cancer patient, or for evaluating the toxicity of a drug, or for quantifying the appropriate dose of a drug for a patient. Briefly, blood from the cancer patient is divided into a control test tube and an assay test tube. The blood in the assay test tube is exposed to a particular drug. The assay test tube and the control test tube are then compared to each other to determine the effect of the drug on cancer cells or other rare cells in the patient's blood. This provides information to a physician on whether the particular drug may be beneficial to the cancer patient or may continue to benefit the patient.

In some embodiments, a method of testing for drug susceptibility in a cancer patient comprises dividing a blood sample of the cancer patient into a control test tube and an assay test tube. A drug is added to the assay test tube. A separator float is introduced into the assay test tube and moved into alignment with the cancer cells to capture the cancer cells in an annular volume. The assay test tube is then visually examined. The effect of the drug on cancer cells in the assay test tube is compared to cancer cells in the control test tube.

The change in the shape of the cancer cells, or the number of intact cancer cells, may be compared between the assay test tube and the control test tube.

The method may further comprise staining the cancer cells prior to visually examining the assay test tube. In addition, the control test tube may be visually examined. For example, the visual examination may detect the quantity of fluorescence in the assay test tube due to the staining (e.g. immunofluorescence).

The visual examination can be performed by introducing a separator float into the assay test tube. The assay test tube is then centrifuged to move the float into alignment with the cancer cells. Subsequently, the rotational speed is reduced or stopped) to capture the cancer cells in a volume between the test tube and the separator float. The cancer cells can then be examined. The visual examination can also be performed using an optical system that generates light having a non-uniform spatial distribution.

In particular embodiments, the separator float comprises a main body portion, a plurality of axially oriented ridges protruding from the main body portion, and does not have end sealing ridges.

In other embodiments, a method of testing for drug susceptibility in a cancer patient, comprises dividing a blood sample from the cancer patient into a control test tube and an assay test tube. A drug is added to the assay test tube. The assay test tube is visually examined to determine the effect of the drug on cancer cells in the blood. The cancer cells in the assay test tube are then compared with the cancer cells in the control test tube.

Another method of testing for drug susceptibility in a cancer patient comprises receiving a first test tube and a second test tube, each tube containing the blood of the cancer patient. A drug is added to the first test tube to make an assay test tube. The assay test tube is visually examined. The effect of the drug on cancer cells in the assay test tube is compared with cancer cells in the control test tube.

Still another method of testing for drug susceptibility in a cancer patient, comprises receiving a blood sample of the cancer patient and dividing the blood sample into a control test tube and an assay test tube. The blood in the assay test tube is mixed with a drug. The assay test tube is visually examined to determine the effect of the drug on a cell type in the blood. The effect on the cell type in the assay test tube is compared with the cell type in the control test tube.

Yet another method of testing for drug susceptibility in a cancer patient comprises dividing a blood sample of the cancer patient into a control test tube and an assay test tube. The blood in the assay test tube is exposed to a drug. The assay test tube is visually examined to determine the effect of the drug on a cell type in the blood. The effect on the cell type in the assay test tube is compared with the cell type in the control test tube.

Another method of testing for drug susceptibility in a cancer patient comprises dividing a blood sample of the cancer patient into a control test tube and a series of assay test tubes. The blood in the assay test tubes is exposed to a drug, with the quantity of the drug varying between assay test tubes. The assay test tubes are visually examined to determine the effect of the amount of the drug on a cell type in the blood. This can help determine the effective dose of the drug, in addition to whether or not the drug is effective.

Sometimes, when the separator float and test tube are used, the cancer cells can be isolated or extracted from the annular volume and further processed.

Disclosed herein are methods of separating and axially expanding buffy coat constitutents in a blood sample; detecting target cells in a blood sample; and capturing or extracting buffy coat constitutents/target cells in a blood sample. Those methods require introducing the blood sample and a rigid volume-occupying float into a flexible sample tube. The rigid float has a specific gravity intermediate that of red blood cells and plasma, and comprises a main body portion spacedly surrounded radially by the sidewall of the sample tube to form an annular volume therebetween; and one or more support members protruding from the main body portion and engaging the sidewall. The sample tube is centrifuged at a rotational speed that causes enlargement of the sidewall to a diameter sufficiently large to permit axial movement of the float, separation of the blood into discrete layers, and movement of the float into alignment with at least the buffy coat constituents of the blood sample. The rotational speed is reduced to cause the sidewall to capture the float and trap buffy coat constituents in the annular volume, which might be divided into one or more analysis areas.

Some methods disclosed herein further comprise welding at least one of the one or more support members to the sidewall.

Disclosed in further embodiments is a sample tube for holding a sample. The sample tube comprises a sidewall, which has a first cross-sectional inner diameter, and interior surface, and an exterior surface. One or more circumferential notches, cuts, or indentations are made on the sidewall of the sample tube to facilitate the breaking, splitting, or separation of the tube at each notch. Usually, the notch is a V-shaped or U-shaped depression in the surface of the sidewall; however, other configurations are also contemplated.

The circumferential notches can be located on the exterior surface or the interior surface of the sidewall of the sample tube. The notches can also be continuous around the circumference, or discontinuous. In particular embodiments, the one or more circumferential notches comprise two sets of notches that divide the tube into three volumes.

In methods using the sample tube with circumferential notches, after centrifugation, the sample tube is broken at at least one of the one or more notches to obtain a broken or isolated section of the tube containing the float and expanded buffy coat constituents.

The one or more circumferential notches can comprise two sets of notches that divide the tube into three volumes. Desirably, one set of notches is above the float and one set of notches is below the float after reducing the rotational speed. No broken notches should be made or be present along the axial length of the float.

Also disclosed in embodiments is a sample tube comprising a cylinder which has a first open end and a second open end. Two closure devices are provided for sealing the two ends.

In methods using the sample tube with two closure devices, the closure devices are removed after centrifugation. This allows red blood cells and plasma to be emptied from the sample tube to isolate the float and expanded buffy coat layer. Examples of such closure devices include removable fitted or screw type cpas, but other closure devices are also contemplated.

Further disclosed are some methods wherein at least a portion of the buffy coat constituents contained in the annular volume are removed through the sidewall of the sample tube using a removal device.

Different float designs are also provided herein. In some embodiments, a volume-occupying separator float has a specific gravity intermediate that of red blood cells and plasma. The float comprises a main body portion having a top end and a bottom end; and one or more support members protruding from the main body portion. The main body portion and the one or more support members define an annular volume. The main body portion also contains a septum for receiving a pitot tube, the septum extending from a top end of the main body portion to the annular volume.

In other embodiments, the separator float includes a pitot tube having a distal end, wherein the pitot tube engages the septum at the top end, and wherein the distal end is located away from the top end of the main body portion.

In methods using such floats having a septum, the septum is engaged with the pitot tube. At least a portion of the buffy coat layer is removed from the annular volume through the pitot tube.

Additionally disclosed are different volume-occupying separator floats. These floats comprise a main body portion; a top support member extending radially from a top end of the main body portion; and a bottom support member extending radially from a bottom end of the main body portion. An annular volume is defined by the main body portion, the top support member, and the bottom support member. One or more intermediate support members extend radially from the main body portion to form a plurality of wells in the annular volume. A plurality of septums is present within the main body portion, each septum allowing access to a particular well from the top end of the main body portion.

In some further embodiments, the one or more intermediate support members consist of a plurality of axially oriented ridges. In others, the one or more intermediate support members consist of a plurality of circumferentially oriented ridges. In still others, the one or more intermediate support members consist of a plurality of axially oriented ridges intersecting with a plurality of circumferentially oriented ridges.

In still other embodiments, a volume-occupying separator float comprises a main body portion having a top end and a bottom end; a bottom support member extending radially from the bottom end of the main body portion; and a plurality of ridges extending radially from the main body portion and extending axially between the top end of the main body portion and the bottom support member to form at least one axially extending flute. The float may consist of one axially extending flute or a plurality of axially extending flutes. At least a portion of the buffy coat constituents can be extracted from such flutes using an extraction device, like a syringe.

Other methods of using a float with a flexible sleeve are also disclosed herein. Generally, the blood sample and float are introduced into the flexible sleeve, then centrifuged. The sleeve is used to seal at least one of the wells to trap the buffy coat constituents in wells in the float.

In some embodiments, the sleeve comprises a sidewall, the sidewall having a polygonal cross-sectional shape with n sides. The float also has n sides. After centrifugation, the sleeve shrinks and attaches to the float, trapping at least a portion of the buffy coat constituents in the n wells. For example, such a sleeve can be triangular in a lateral cross-sectional configuration (i.e. n=3), square (i.e n=4), pentagonal (i.e. n=5), etc.

Some floats described herein comprise a main body portion having a top end, a bottom end, and n sides, wherein n is an integer greater than two. A top support member extends laterally away from the top end of the main body portion, and a bottom support member extends laterally away from the bottom end of the main body portion. A plurality of ridges is also present, each ridge extending laterally away from the main body portion and extending axially from the top support member to the bottom support member to form n axially-oriented wells, each well having an exterior surface. The float is adapted to be unfolded so that the exterior surfaces of the wells can lie substantially in the same plane.

Some volume-occupying floats disclosed herein comprise a main body portion having a top end and a bottom end. One or more support members protrude from the main body portion, and a hollow internal cavity is present within the main body portion. The main body portion and the one or more support members define an annular volume, and one or more one-way valves permit flow from the annular volume to the hollow internal cavity. A plug may be present at the top end of the main body portion for accessing the hollow internal cavity.

Another similar volume-occupying separator float comprises two main body portions. A first main body portion comprises a sidewall that defines a central bore, the central bore being accessible from a top end. A bottom support member extends radially from a bottom end of the sidewall. A first thread is located within the central bore. One or more one-way valves are located in the sidewall and directed to permit entry of fluid into a bottom end of the central bore. The second main body portion comprises a center portion sized to fit within the central bore and a complementary thread located on the center portion for engaging the first thread of the first main body portion. A top support member extends radially from a top end of the second main body portion. This float operates by being unscrewed to increase the volume in the central bore.

This float may further comprise a plug at the top end of the second main body portion for accessing the central bore. This float also generally comprises a keyhole in the second main body portion for unscrewing the second main body portion from the first main body portion.

When the second main body portion is unscrewed, the second main body portion moves upward, reducing the pressure in the central bore, and evacuating at least a portion of the buffy coat constituents into the central bore. A fluid can subsequently be bled into the annular volume.

Another volume-occupying separator float also comprises two main body portions. The first main body portion comprises a cylinder that defines a central bore, the central bore being accessible from a top end. A bottom support member extends radially from a bottom end of the cylinder. One or more one-way valves located in the cylinder are directed to permit entry of fluid into a bottom end of the central bore. The second main body portion comprises a center portion sized to slidably fit within the central bore, and a top support member extending radially from a top end of the second main body portion.

Disclosed in some embodiments is a volume-occupying separator float comprising at least a first piece and a second piece. Each piece has a first end, a second end, an exterior surface, and an interior surface. The interior surfaces of the first and second pieces cooperate to form an open passage extending between the first end and the second end. The first and second pieces can be connected together.

The first and second pieces may have substantially the same three-dimensional shape. In some embodiments, the interior surface of the first piece comprises a semi-cylindrical surface. Put another way, a lateral cross-sectional view of the first piece may have a semi-annular shape. Sometimes, the interior surface of the first piece is substantially planar, i.e. a lateral cross-sectional view of the interior surface of the first piece is substantially a straight line. In some floats, a lateral cross-sectional view of the interior surface of the first piece is substantially a straight line, and a lateral cross-sectional view of the interior surface of the second piece is substantially a straight line with a central indent. In specific embodiments, a lateral cross-sectional view of the open passage may have a rectangular shape or a circular shape.

The exterior surface of the first and second pieces may each substantially conform to an inner surface of the sidewall of the sample tube in which the two-piece float is used. The exterior surface of the first and second pieces may also each comprise at least one support member for engaging an inner surface of the sidewall.

The first and second pieces can be joined together using clips, clamps, and other joining mechanisms or devices.

Sometimes, the first and second piece each further comprise at least one side surface. The first and second pieces can be connected together on the at least one side surface.

The two-piece or multiple-piece float can be used in conjunction with a flexible bag to capture buffy coat constituents in a blood sample. A blood sample is introduced into a flexible bag, and a float is placed around the flexible bag, then the bag and float are placed in a flexible sample tube. During centrifugation, the float moves into alignment with at least the buffy coat constituents. Upon reducing the rotational speed, the sidewall captures the float. The flexible bag can then be sealed at the first end and the second end of the float to capture the buffy coat constituents. The flexible bag can be sealed by welding the first end and the second end of the float. The welding may be performed ultrasonically. Other sealing and/or enclosure devices or mechanisms are also contemplated.

Disclosed in other embodiments is a volume-occupying separator float comprising a main body portion. The main body portion has a first end and a second end and at least one pressure seal. A buffy coat passage extends from the second end to the first end and has a centrifugation valve oriented to open during centrifugation, the valve being located at the second end. In some specific embodiments, there is a first pressure seal around the first end and a second pressure seal around the second end of the main body portion. In additional embodiments, a pressure relief passage extends from the second end to the first end and has a pressure relief valve oriented to open when pressure at the second end is greater than pressure at the first end by a specified value. In other embodiments, there is a pressure relief valve but not a buffy coat passage in the separator float.

During centrifugation, the pressure seal(s) substantially prevent the blood sample from traveling between the float and the inner surface of the sample tube. The buffy coat constituents are trapped in the buffy coat passage, and the float can then be removed from the sample tube to obtain the buffy coat constituents.

Alternatively, a volume-occupying separator float comprises a main body portion. The main body portion has a first end and a second end. One or more centrifugation valves are circumferentially disposed about the main body portion.

Disclosed in still other embodiments is a volume-occupying separator float comprising a first piece and a second piece. The first and second pieces cooperate to form a rectangular passage between a first end and a second end of the float, and wherein the first and second pieces are joined at the first end of the float. A slide can be placed or located in the rectangular passage.

During use, this two-piece float containing a slide is placed in a sample tube and centrifuged. This causes the slide to be coated with the buffy coat constituents of the blood sample. The float can then be removed from the sample tube; and the slide can be extracted from the float. This float can also be used to surround a flexible bag, as described above.

In other embodiments, a two-piece float comprises a top float and a bottom float. The top float has a density intermediate that of plasma and the buffy coat constituents. The top float comprises (i) a lower support member having an upper surface and a lower surface, and (ii) a pitot tube extending axially from the lower surface of the top float through the upper surface and having a top end located distally from the upper surface. The bottom float has a density intermediate that of the buffy coat constituents and red blood cells. The top float lower support member lower surface and the bottom float are complementarily shaped.

The two-piece float may be designed to relieve pressure below the bottom float. The top float further comprises a second passage extending from the lower surface to the upper surface of the lower support member. The bottom float comprises a support member having an upper surface and a lower surface. A pressure relief tube extends axially from the lower surface of the bottom float support member through the upper surface and terminates at an upper end. The upper end of the bottom float pressure relief tube extends through the top float second passage.

The top float may further comprise a manipulator extending axially from the upper surface of the lower support member. The manipulator is used to push the top float towards the bottom float. The manipulator can be considered a handle for handling the top float.

During centrifugation, the buffy coat constituents become aligned between the top float and the bottom float. The top float is then pushed toward the bottom float to remove the buffy coat constituents through the pitot tube.

The pitot tube may be integral, or made as a separate piece. When the pitot tube is separate, the top float includes (ii) a first passage from the lower surface of the lower support member to the upper surface of the lower support member. After centrifugation, the pitot tube engages the first passage of the top float, and the top float can then be pushed toward the bottom float to remove the buffy coat constituents through the pitot tube.

Still other embodiments of a two-piece float comprise a top float and a bottom float. The top float has a density intermediate that of plasma and the buffy coat constituents. The top float comprises (i) a lower lateral support member having an upper surface and a lower surface, and (ii) a manipulator extending axially from the upper surface of the lower lateral support member. The bottom float has a density intermediate that of the buffy coat constituents and red blood cells. The bottom float comprises an upper lateral support member having an upper surface and a lower surface. The top float lower lateral support member and the bottom float upper lateral support member are complementarily shaped to form a recess. The recess can be used to trap the buffy coat constituents, and/or can enclose a slide.

The bottom float may further comprise a support member extending axially from the lower surface of the upper lateral support member.

The recess can be substantially formed in the bottom float upper lateral support member, with the top float lower lateral support member covering the recess. Alternatively, the recess can be formed in the top float lower lateral support member and the bottom float upper lateral support member covering the recess.

During use, the buffy coat constituents become aligned between the top float and the bottom float. The top float is then pushed toward the bottom float to push the buffy coat constituents into the recess, and coat the slide. The two-piece float and the sample tube can be separated, and the buffy coat constituents or the slide can then be removed from the two-piece float.

In other embodiments, the top float comprises (i) a lower lateral support member having an upper surface and a lower surface, and (ii) a hollow member open on the lower surface of the lower lateral support member and extending axially from the upper surface of the lower lateral support member. The hollow member can be adapted to receive a slide. The bottom float comprises an upper lateral support member for sealing the hollow member.

Also disclosed in embodiments are additional designs for a volume-occupying separator float. The float comprises a main body portion having a top end and a bottom end. One or more support members protrude laterally from the main body portion. The main body portion and the one or more support members define an annular volume. A pitot tube extends axially from the top end of the main body portion. An internal passage passes through the main body portion and connects the pitot tube to an opening in the annular volume. An upper piece comprises (i) a passageway through which the pitot tube extends, and (ii) a manipulator extending axially away from the main body portion. The upper piece has a lower density than the main body portion. The manipulator acts as a handle for moving the upper piece.

The upper piece passageway can be located inside the manipulator.

The float may further comprise a one-way valve in the opening oriented to permit flow from the annular volume into the internal passage. The main body portion internal passage can also connect the pitot tube to a plurality of openings in the annular volume.

The main body portion may further comprise a pressure relief passage extending from the first end to the second end, the pressure relief passage not intersecting the internal passage.

In particular embodiments, one support member is located on the bottom end of the main body portion, and the opening connecting to the annular volume is located proximally to the one support member.

In other embodiments, the float comprises one support member extending laterally from a bottom end of the main body portion and at least one helical support member located proximal to the top end of the main body portion.

In use, the upper piece is pushed down to force fluid towards the annular volume and push the buffy coat constituents through the pitot tube. These embodiments contemplate the pitot tube being integral with the main body portion.

Sometimes, the pitot tube is separate from the main body portion. Then, only the main body portion is placed in the tube with the blood sample. Centrifugation causes alignment of the annular volume with at least the buffy coat constituents. The pitot tube is then engaged with the internal passage at the top end of the main body portion. The upper piece, comprising a hollow member that extends axially away from the main body portion and surrounds the pitot tube, is threaded around the pitot tube. The upper piece is then pushed down to force fluid towards the annular volume and push the buffy coat constituents through the pitot tube.

In some embodiments, the blood sample and the float are introduced into a flexible sleeve. A compressible material is fed into a sample tube, and the flexible sleeve is placed into the sample tube such that (i) the compressible material is between the sample tube and the flexible sleeve, and (ii) the compressible material applies pressure sufficient to cause the flexible sleeve to engage the float. During centrifugation, the pressure of the compressible material against the flexible sleeve is reduced, permitting movement of the float into alignment with at least the buffy coat constituents of the blood sample. Upon reducing the rotational speed, the compressible material again applies pressure that causes the flexible sleeve to engage the float, trapping the buffy coat constituents in the annular volume.

If desired, the flexible sleeve can be removed from the sample tube. The blood sample present in the annular volume can then be analyzed. Alternatively, at least one support member of the float can be welded to the flexible sleeve.

The compressible material can be water, a slurry, a gel, a foam, or an elastomer. Generally, the compressible material is fed into the sample tube in a volume such that the compressible material is at a level in the sample tube higher than a top end of the main body portion after centrifugation. Desirably, the compressible material has a viscosity low enough so as not to adhere to the flexible sleeve.

In other embodiments, a non-flexible metal sample tube is used. The blood sample and the float are introduced into a flexible sleeve, and the flexible sleeve is placed into the metal sample tube. After centrifugation, the metal sample tube is constricted to capture the float and trap buffy coat constituents in the annular volume.

A kit for separation of buffy coat constituents in a blood sample is also provided. The kit includes a metal sample tube, a flexible sleeve, and a float. The float has a specific gravity intermediate that of red blood cells and plasma. The float has a main body portion and one or more support members protruding from the main body portion.

Disclosed in other embodiments is a flexible volume-occupying separator float. The flexible float comprises a main body portion and one or more support members protruding from the main body portion. Prior to centrifugation, the float has a first cross-sectional diameter. The float is formed from a compressible material such that the float will shrink to a second cross-sectional diameter which is less than the first cross-sectional diameter upon application of a centrifugal force.

In use, the first cross-sectional diameter of the flexible float is sized to engage the sidewall of a sample tube. The sample tube may be flexible or rigid (i.e. non-flexible). The tube is then centrifuged at a rotational speed that causes the float to shrink to the second cross-sectional diameter, which is sufficiently small to permit movement of the float into alignment with at least the buffy coat constituents of the blood sample. Upon reducing the rotational speed, the float enlarges to the first cross-sectional diameter, trapping buffy coat constituents in the annular volume.

Other embodiments of a flexible volume-occupying separator float are also disclosed. There, the float comprises a main body portion made from a flexible sidewall. The flexible sidewall has a first edge and a second edge, the first and second edges overlapping to define an interior volume. The first edge comprises a detent and the second edge comprises a notch. A spring is located within the interior volume, the spring having a first end and a second end. The first end of the spring is attached to an interior surface, and the second end of the spring is attached to the second edge of the flexible sidewall. The spring compresses during centrifugation to reduce the diameter of the float. The detent engages the notch of the second edge when the spring expands after centrifugation.

In use, the spring compresses during centrifugation, reducing the diameter of the float. This shrinkage permits movement of the float into alignment with at least the buffy coat constituents of the blood sample. Upon reducing the rotational speed, the spring expands and the float returns to its original diameter, capturing the buffy coat constituents in the annular volume. The float may be used with a flexible or rigid sample tube.

Other designs for a flexible volume-occupying separator float are also disclosed. The float comprises an inner core, an outer sidewall, and at least one support member connecting the inner core to the outer sidewall. The inner core has a top end and a bottom end. The outer sidewall is formed from an optically clear material. Buffy coat materials become trapped between the inner core and the outer sidewall.

At least one high pressure seal may surround the outer sidewall if desired. In particular embodiments, a top high pressure seal is present around a top end of the outer sidewall, and a bottom high pressure seal is present around a bottom end of the outer sidewall.

The at least one support member may include a plurality of axial ridges that extend axially from the top end of the inner core to the bottom end of the inner core.

In some embodiments, the float also includes a bottom end cap for sealing a bottom end of the float, the bottom end cap having a diameter substantially equal to a diameter of the outer sidewall. The inner core may have an internal passage extending from the bottom end to the top end, so that a manipulator can extend through the internal passage to the bottom end cap for handling the bottom end cap. The bottom end of the inner core and the bottom end cap may comprise a mutual engagement system for connecting the bottom end cap to the inner core.

In other embodiments, the float can include a top end cap for sealing a top end of the float, the top end cap having a diameter substantially equal to a diameter of the outer sidewall. The top end of the inner core and the top end cap may comprise a mutual engagement system for connecting the top end cap to the inner core. The top end cap may have a member extending axially away from the float.

Sometimes, both a top end cap and a bottom end cap are used. In some of these embodiments, the top end cap member is hollow, and the bottom end cap manipulator extends through the top end cap member.

Generally, when the float is used, the buffy coat constituents of the blood sample become located in the annular volume between the outer sidewall and the inner core. The buffy coat constituents can be analyzed through the optically clear outer sidewall of the float.

Alternatively, the bottom end of the float can be sealed with the bottom end cap to capture the buffy coat constituents within the float. The top end of the float can also be sealed with the top end cap.

These and other non-limiting aspects and/or objects of the disclosure are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is a side view of a test tube and a separator float that can be used to visualize rare cells in a blood sample.

FIG. 2 is a diagram of a microscope system that can be used to visualize rare cells in a blood sample.

FIG. 3 is a perspective view of a test tube holder that can be used to with the system of FIG. 2, showing the internal components.

FIG. 4 is a side view of the test tube holder of FIG. 3, showing the internal components.

FIG. 5 is a perspective view of the test tube holder of FIG. 3, showing certain bearings.

FIG. 6 is a top view of the test tube holder of FIG. 3, showing certain internal components.

FIG. 7 is an embodiment of a separator float with end sealing ridges and radial support members.

FIG. 8 is an embodiment of a separator float with radial support members having a rectangular cross-section and no end sealing ridges.

FIG. 9 is an embodiment of a separator float with end sealing ridges and a helical support member having a rectangular cross-section.

FIG. 10 is an embodiment of a separator float with radial support members having a curved cross-section and no end sealing ridges.

FIG. 11 is an embodiment of a separator float with end sealing ridges and a helical support member having a curved cross-section.

FIG. 12 is an embodiment of a separator float with end sealing ridges and axially aligned support members.

FIG. 13 is a cross-sectional view of the float of FIG. 12.

FIG. 14 is an embodiment of a separator float with axially aligned support members and no end sealing ridges.

FIG. 15 is an embodiment of a separator float with end sealing ridges and axially aligned support members.

FIG. 16 is a perspective view of the float of FIG. 15.

FIG. 17 is an embodiment of a separator float with axially aligned support members and no end sealing ridges.

FIG. 18 is an embodiment of a separator float with end sealing ridges, radial ribs, and axially aligned splines.

FIG. 19 is an embodiment of a separator float with end sealing ridges and protrusions as support members, with the protrusions in a staggered pattern.

FIG. 20 is an embodiment of a separator float with protrusions in a staggered pattern and no end sealing ridges.

FIG. 21 is an embodiment of a separator float with end sealing ridges and protrusions as support members, with the protrusions in an aligned pattern.

FIG. 22 is an embodiment of a separator float with protrusions in an aligned pattern and no end sealing ridges.

FIG. 23 is a flowchart of one exemplary embodiment of the methods of the present disclosure.

FIG. 24 is an embodiment of a separator float having conical ends.

FIG. 25 is an embodiment of a separator float having frustoconical ends.

FIG. 26 is an embodiment of a separator float having convex or dome-shaped ends.

FIG. 27 is an embodiment of a separator float having the end sealing ridges offset from the ends of the main body portion.

FIG. 28 is an embodiment of a separator float having faceted protrusions and end sealing ridges.

FIG. 29 is an embodiment of a separator float having faceted protrusions and no end sealing ridges.

FIG. 30 is an embodiment of a separator float having a central bore and conical ends.

FIG. 31 is an embodiment of a separator float having a central bore, conical ends, and radially extending ribs.

FIG. 32 is an embodiment of a separator float having a central bore, conical ends, and axially extending ribs.

FIG. 33 is an exploded perspective view of a two-piece separator float.

FIG. 34 is a cross-sectional view of a two-piece separator float wherein the piston has a flanged end.

FIG. 35 is a cross-sectional view of a two-piece separator float having a flanged end and including tapered ends.

FIG. 36 is a cross-sectional view of a two-piece separator float which includes a central bore and a counterbore having different diameters.

FIG. 37 is a cross-sectional view of a two-piece separator float with a central bore and a counterbore having different diameters, and also having tapered ends.

FIG. 38 is a cross-sectional view of a two-piece separator float having a profiled bore and an enlarged head that interact.

FIG. 39 is a cross-sectional view of a two-piece separator float having a tapered internal passage.

FIG. 40 is a cross-sectional view of a two-piece separator float having an annular seat for the piston.

FIG. 41 is a diagram of a microscope system similar to that of FIG. 2, but with a modified optical system.

FIG. 42 is a diagram of a microscope system similar to that of FIG. 2, but with another modified optical system.

FIG. 43 is a diagram of a microscope system similar to that of FIG. 2, but with yet another modified optical system.

FIG. 44 is a top view of another test tube holder like FIG. 3, but using a test tube with an eccentric cross-section.

FIG. 45 is a top view of another test tube holder like FIG. 3, but using a test tube with an eccentric cross-section.

FIG. 46 is a side view of a portion of a test tube holder employing tilted roller bearings.

FIG. 47 is a side view of a portion of a test tube holder employing tilted roller bearings staggered along a test tube axis, along with a float having helical ridges enables spiral scanning of the test tube.

FIG. 48 is a perspective view of a test tube holder that holds the test tube horizontally and uses the test tube as a bias force.

FIG. 49 is a top view of a test tube holder employing bushing surfaces as alignment bearings and a set of ball bearings as bias bearings.

FIG. 50 diagrammatically depicts certain measurement parameters relevant in performing quantitative buffy coat analysis using a buffy coat sample trapped in an annular gap between an inside test tube wall and an outer surface of a float.

FIG. 51 diagrammatically shows a suitable quantitative buffy coat measurement/analysis approach.

FIG. 52 diagrammatically shows another suitable quantitative buffy coat measurement/analysis approach.

FIG. 53 diagrammatically shows a suitable image processing approach for tagging candidate cells.

FIG. 54 shows a pixel layout for a square filter kernel suitable for use in the matched filtering.

FIG. 55 shows a pixel intensity section A-A of the square filter kernel of FIG. 54.

FIG. 56 diagrammatically shows a suitable user verification process for enabling a human analyst to confirm or reject candidate cells.

FIG. 57 is a diagram illustrating the methods of the present disclosure.

FIG. 58A is a side view of a notched sample tube containing a volume-occupying separator float therein.

FIG. 58B is a perspective view of a continuous notch on a notched sample tube.

FIG. 58C is a perspective view of a discontinuous notch on a notched sample tube.

FIG. 58D is a side view of a rectangular notch on a notched sample tube.

FIG. 58E is a side view of a triangular notch on a notched sample tube.

FIG. 59 is a side view of an exemplary sample tube having two open ends which are each sealed with a closure device and containing a volume-occupying separator float therein.

FIG. 60 is a side view of an apparatus comprising a sample tube, a volume-occupying separator float within the sample tube, and a syringe penetrating the sidewall of the sample tube to access the annular volume.

FIG. 61 is a side view of a sample tube containing a volume-occupying separator float that has a pitot tube extending through the float to access the annular volume.

FIG. 62 is a perspective view of a volume-occupying separator float having axial intermediate support members that form axial wells.

FIG. 63 is a side view of a volume-occupying separator float having circumferential intermediate support members that form circumferential.

FIG. 64 is a perspective view of a volume-occupying separator float having axial intermediate support members and circumferential intermediate support members that intersect to form a plurality of wells.

FIG. 65 is a top perspective view of a volume-occupying separator float having a single axial flute.

FIG. 66 is a top perspective view of a volume-occupying separator float having a plurality of axial flutes.

FIG. 67 is a cross-sectional view of a portion of a flexible sleeve containing a volume-occupying separator float therein.

FIG. 68 is a perspective view of a flexible sleeve containing a volume-occupying separator float with a square cross-section.

FIG. 69 is a perspective view of a volume-occupying separator float which has been unfolded.

FIG. 70 is a side view of a volume-occupying separator float with a one-way valve permitting flow from the annular volume of the float into a hollow internal cavity.

FIG. 71 is a side view of another volume-occupying separator float. The float has two pieces which are threaded together.

FIG. 72 is a side view of another volume-occupying separator float. The float has two pieces which are slidably engaged.

FIG. 73 is a side view of another volume-occupying separator float. The float has sharp support members.

FIG. 74A is a side cross-sectional view of a sample tube containing a separator float surrounding a flexible bag.

FIG. 74B is a top cross-sectional view of a first exemplary embodiment of a two-piece float for surrounding a flexible bag.

FIG. 74C is a top cross-sectional view of a second exemplary embodiment of a two-piece float for surrounding a flexible bag.

FIG. 74D is a top cross-sectional view of a third exemplary embodiment of a two-piece float for surrounding a flexible bag.

FIG. 75A is a side cross-sectional view of a first exemplary embodiment of a separator float containing a buffy coat passage for trapping/catching the buffy coat constituents and using a centrifugation valve to control flow through the buffy coat passage.

FIG. 75B is a side cross-sectional view of a second exemplary embodiment of a separator float containing a buffy coat passage for trapping/catching the buffy coat constituents and using a centrifugation valve to control flow through the buffy coat passage.

FIG. 75C is a side cross-sectional view of a third exemplary embodiment of a separator float using a centrifugation valve to control flow through an annular volume.

FIG. 75D is a side cross-sectional view of another exemplary embodiment of a separator float.

FIG. 76A is a side cross-sectional view of a two-piece separator float containing a slide.

FIG. 76B is a perspective view of the float of FIG. 76A in a closed position.

FIG. 76C is a perspective view of the float of FIG. 76A in an open position.

FIG. 77 is a side cross-sectional view of a two-piece float that traps buffy coat constituents and removes them through a pitot tube.

FIG. 78A is a side cross-sectional view of a first exemplary embodiment of a two-piece float that traps buffy coat constituents in a recess.

FIG. 78B is a side cross-sectional view of a second exemplary embodiment of a two-piece float that traps buffy coat constituents in a recess.

FIG. 78C is a side cross-sectional view of a third exemplary embodiment of a two-piece float that traps buffy coat constituents.

FIG. 79 is a side cross-sectional view of a first exemplary embodiment of a two-piece float for extracting buffy coat constituents.

FIG. 80 is a side cross-sectional view of a second exemplary embodiment of a two-piece float for extracting buffy coat constituents.

FIG. 81 is a side view of a sample tube containing a compressible material, a flexible sleeve, and a separator float.

FIG. 82 is a side view of a metal sample tube containing a flexible sleeve and a volume-occupying separator float therein.

FIG. 83 is a side view of a rigid sample tube containing a flexible or compressible separator float.

FIG. 84 is a top cross-sectional view of a flexible separator float formed from a flexible sidewall.

FIG. 85 is a side view of a notched sample tube containing a separator float having a top end cap and a bottom end cap.

FIG. 86 is a perspective view of the separator float of FIG. 85.

### DETAILED DESCRIPTION

A more complete understanding of the processes and apparatuses disclosed herein can be obtained by reference to the accompanying drawings. These figures are merely schematic representations based on convenience and the ease of demonstrating the existing art and/or the present development, and are, therefore, not intended to indicate relative size and dimensions of the assemblies or components thereof.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The modifier “about” used in connection with a quantity is inclusive of the stated value and has the meaning dictated by the context (for example, it includes at least the degree of error associated with the measurement of the particular quantity). When used in the context of a range, the modifier “about” should also be considered as disclosing the range defined by the absolute values of the two endpoints. For example, the range of “from about 2 to about 10” also discloses the range “from 2 to 10.”

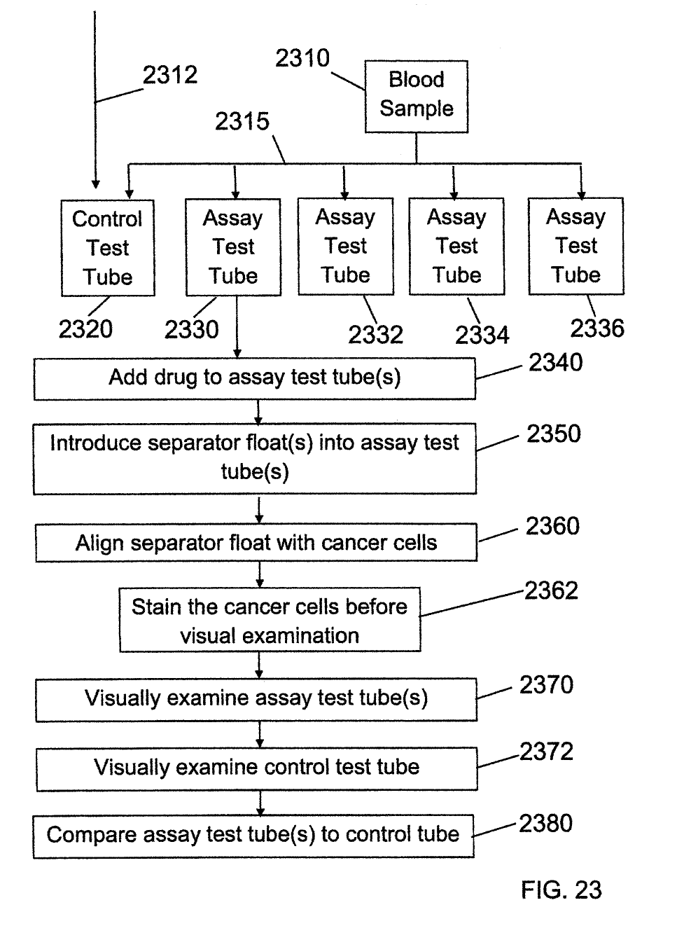

The present disclosure relates to a test for drug susceptibility using at least two test tubes and a rare cell detection system. The test is used to help determine whether a given drug will be useful for treating a cancer patient, and perhaps determining how much drug will be useful, before the given drug is actually administered to the patient. The drug is administered to at least one test tube (“the assay test tube”) and the other test tube is used as a “control test tube”. A series of assay test tubes which vary in the dose or concentration of the drug can also be used. The assay test tube(s) are then visually examined to determine the impact on the circulating cancer cells or other cells in the blood sample. This provides insight into the potential efficacy of the administered drug, and can also indicate an effective concentration or dose, and could also be used during drug research to identify appropriate drug candidates and their potential effective dose(s). FIG. 23 is a flowchart illustrating the test and its various steps. It should be noted that this is only one order in which the steps can be performed, and other orders of these steps are contemplated, as may be described further herein.

Initially, a control test tube and at least one assay test tube are obtained, each test tube containing blood from the cancer patient. Different methods for preparing the two test tubes are contemplated. For example, a blood sample of the cancer patient could be received for testing 2310. The blood sample can be procured from the cancer patient using normal procedures. The blood sample can be received in the form of one large sample that is subsequently divided 2315 into at least two smaller samples, corresponding to the control test tube 2320 and one or more assay test tubes 2330. Alternatively, the blood sample can be received in the form of two or more test tubes (i.e. at least a first test tube and a second test tube), which can be considered as having been divided prior to receipt, and as corresponding to the control test tube and the assay test tube (marked with reference numeral 2312).

Next, a drug 2340 is added to the assay test tube(s). Depending on the methods of the present disclosure, the drug may be one that is already known to have anti-cancer activity, or is a candidate being tested for anti-cancer activity. The assay test tube(s) can be shaken, mixed, or otherwise manipulated to ensure that the drug contacts or reacts with the various types of cells in the blood. In this regard, whole blood contains many different types of cells, including packed red cells, reticulocytes, granulocytes, lymphocytes/monocytes, platelets, and plasma, which can be separated by density. The blood of a cancer patient can also include circulating tumor cells and other rare cells of interest, such as certain epithelial cells which are associated with a specific type of cancer.

The assay test tube(s) are then visually examined 2370. The control test tube is also visually examined 2372, so that the results of the test tubes can be compared to each other 2380. The order in which the test tubes are examined is not important. This visual examination allows the effect of the drug on cancer cells in the blood to be determined. The effects provide information on the potential efficacy (and effective concentration) of the drug. The visual examination can be directed, for example, to the morphology of a given cell type in the blood sample or changes in the shape of the given cell. Alternatively, the visual examination might be for lysis of a given cell type, or put another way the quantity of intact cancer cells. As another example, the blood sample can be stained 2362 with an immunofluorescent agent that identifies specific cells or cell types, and the visual examination is then conducted to locate and examine those cells or cell types. The term “visual” refers to an examination of the test tubes using information gathered by light, rather than other means such as radioactivity or electrical patterns. For example, visual examination can be carried out using the human eye or a microscope to inspect the test tube and the cells contained within.

In this regard, immunofluorescence is a technique that uses the specificity of antibodies to their antigen to attach fluorescent dyes to specific biomolecule targets within a cell, and therefore allows visualisation of the target molecules in the sample. Primary, or direct, immunofluorescence uses a single antibody that is chemically linked to a fluorophore. The antibody recognizes the target molecule and binds to it, and the fluorophore it carries can be detected via microscope. Secondary, or indirect, immunofluorescence uses two antibodies. The first or primary antibody recognises the target molecule and binds to it. The second or secondary antibody carries the fluorophore and binds to the primary antibody. Alternatively, the drug can be a fluorescently labeled drug, whose presence in the cell could then be visualized. Methods of fluorescent labeling a drug and such labeled drugs are known in the art. The quantity of fluorescence could be detected and/or measured using visual examination. The fluorescence can be measured in bulk or locally (e.g. between layers).

In this regard, it is known, for example, that a drug having an amine functional group can be reacted with dansyl chloride to produce stable fluorescent sulfonamide adducts. Known fluorophores include rhodamine, coumarin, fluorescein, and cyanine, and derivatives thereof. These fluorophores can be modified to label a drug. For example, a fluorescent dye iodoacetamide can be used to label a drug having a thiol functional group.

It is particularly contemplated that the visual examination of the assay test tube can be enhanced by using a separator float system. Briefly, this includes introducing a separator float 2350 into the assay test tube(s), and moving the separator float into alignment with the cancer cells 2360 in the test tube to capture those cancer cells in an annular volume, usually by centrifugal spinning of the test tube(s). This makes it easier to visualize the cancer cells. See FIG. 23.

It should be noted that the staining to enhance the visual examination (e.g. with a immunofluorescent agent) can be performed prior to introducing the separator float, or can be performed after the spinning of the test tube(s). For example, in FIG. 23, step 2362 may occur before step 2350 if desired. Similarly, the addition of the drug to the assay test tube(s) (step 2340) can be performed before or after introducing the separator float (step 2350), though the drug is usually added before the spinning of the tube/alignment of the separator float,

Specific embodiments are contemplated wherein more than one assay test tube is used. Different quantities (amount or concentration) of the drug can be added between different assay test tubes. This would permit a quantitative determination of the effective dose (if any) of the drug, rather than just a qualitative determination (works or does not work). As an example, four assay test tubes 2330, 2332, 2334, 2336 could be used, containing 1 mg/ml, 2 mg/ml, 4 mg/ml, and 8 mg/ml of the drug, respectively (see FIG. 23).

The testing conditions may vary, depending on the drug being tested. For example, the temperature of the test tubes can vary between room temperature (23-25° C.) to 37° C. The time for which the assay test tube(s) is exposed to the drug before visual examination may vary from minutes to hours. The pH of the liquid in the test tubes is likely to be maintained close to physiological (pH=7.4), but could vary from pH 7.1 to 7.6, or more ideally from pH 7.2 to 7.55.

Some specific systems are contemplated for use in visual examination of the two test tubes used in the methods of the present disclosure. One system is a buffy coat separator float system as described in U.S. patent application Ser. No. 10/263,974, the entirety of which is hereby incorporated by reference herein.

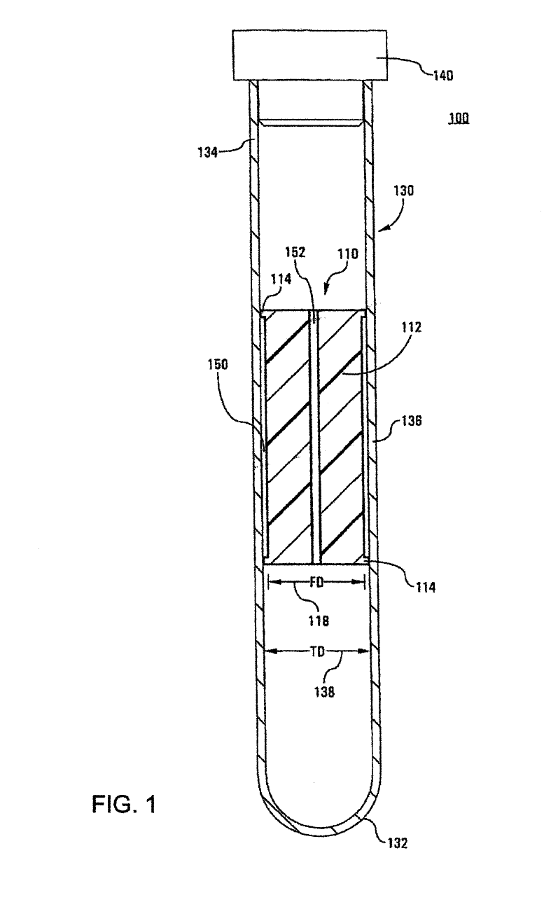

FIG. 1 shows a blood separation tube and float assembly 100, including a test tube 130 having a separator float 110, which can be used in the methods of the present disclosure. The test tube 130 is generally cylindrical. However, the tube 130 may be minimally tapered, slightly enlarging toward the open end 134, particularly when manufactured by an injection molding process. This taper or draft angle is generally desirable for ease of removal of the tube from the injection-molding tool. The test tube 130 includes a first, closed end 132 and a second open end 134 receiving a stopper or cap 140. Other closure means are also contemplated, such as parafilm or the like. Sample tubes having polygonal and other geometrical cross-sectional shapes are also contemplated. In other words, the sample tube may have a cross-section that is a polygon having n sides. For example, when n=3, the sample tube has a triangular cross-section. In particular, the sample tube may have a regular polygonal cross-section (i.e. the lengths of each side are substantially equal).

The tube 130 is formed of a transparent or semi-transparent material sufficient for visual examination. The sidewall 136 of the tube 130 is sufficiently flexible or deformable such that it expands in the radial direction during centrifugation, e.g., due to the resultant hydrostatic pressure of the sample under centrifugal load. As the centrifugal force is removed, the tube sidewall 136 substantially returns to its original size and shape.

The tube may be formed of any transparent or semi-transparent, flexible material. Preferably, the tube material is transparent. However, the tube does not necessarily have to be clear, as long as the receiving instrument that is looking for the cells or items of interest in the sample specimen can “see” or detect those items in the tube.

Preferably, the tube 130 is sized to accommodate the float 110 plus at least about five milliliters of blood or sample fluid, more preferably at least about eight milliliters of blood or fluid, and most preferably at least about ten milliliters of blood or fluid. In an especially preferred embodiment, the tube 130 has an inner diameter 138 of about 1.5 cm and accommodates a volume of from about two milliliters to about ten milliliters of blood in addition to the float 110.

The float 110 includes a main body portion 112 and one or more support members 114. In the embodiment shown here, the support members are seen as two sealing rings or flanges disposed at opposite axial ends (i.e. first and second ends, or top and bottom ends) of the float 110. The float 110 is formed of one or more generally rigid organic or inorganic materials, preferably a rigid plastic material. In this regard, the use of materials and/or additives that interfere with the visual examination method should be avoided. For example, if fluorescence is used, the material utilized to construct the float 110 should not have much “background” fluorescence at the wavelength of interest.

The main body portion 112 has an outer diameter 118 which is less than the inner diameter 138 of the test tube 130, under pressure or centrifugation. The support members 114 of the float generally have a diameter corresponding to the inner diameter 138 of the test tube 130. The main body portion 112 of the float, the support members 114, and the sidewall 136 of the tube 130 thereby define an annular channel or gap 150. The main body portion 112 occupies much of the cross-sectional area of the tube, the annular gap 150 being large enough to contain a specified portion of the blood sample. Preferably, the dimensions 118 and 138 are such that the annular gap 150 has a radial thickness ranging from about 25-250 microns, most preferably about 50 microns. It should be noted that the term “annular” is used to refer to the ring-like shape formed by the float within the tube, and should not be construed as requiring the shape to be defined by two concentric circles. Rather, the tube and the float may each have different shapes and “annular” refers to the shape formed between them. The number of support members 114 may also vary, as will be seen further herein.

An optional bore or channel 152 may extend axially through the float 110. In this regard, the tube/float system can be centrifuged to separate the blood components by density. During centrifugation, the tube expands, freeing the float in the blood sample. As centrifugation is slowed, the float is captured by the wall 136 of the tube as it returns to its original diameter. As the tube continues to contract, pressure may build up in the blood fraction trapped below the float, primarily red blood cells. This pressure may cause cells to be forced into the annular channel 150 containing the captured blood components, thus making imaging of the contents of the annular channel more difficult. The bore 152 allows for any excessive fluid flow or any resultant pressure in the dense fractions trapped below the float 110 to be relieved. The excessive fluid flows into the bore 152, thus preventing degradation of the captured blood components. The bore extends completely from one end of the float to the other. In the preferred embodiment, the bore 152 is centrally located and extends axially.

While in some instances the outer diameter 118 of the main body portion 112 of the float 110 may be less than the inner diameter 138 of the tube 130, this relationship is not required. This is because once the tube 130 is centrifuged (or pressurized), the tube 130 expands and the float 110 moves freely. Once the centrifugation (or pressurization) step is completed, the tube 110 constricts back down on the sealing rings or support ridges 114 to capture the float. The annular volume 150 is then created, and sized by the length of the support ridges or sealing rings 114 (i.e., the depth of the “pool” is equal to the length of the support ridges 114, independent of what the tube diameter is/was).

In desired embodiments, the float dimensions are 3.5 cm tall×1.5 cm in diameter, with a main body portion sized to provide a 50-micron gap for capturing the buffy coat layers of the blood. Thus, the volume available for the capture of the buffy coat layer is approximately 0.08 milliliter. Since the entire buffy coat layer is generally less than about 0.5% of the total blood sample, the preferred float accommodates the entire quantity of buffy layer separated in a two to ten milliliter sample of blood.

As previously stated, the support members 114 are sized to be roughly equal to, or slightly greater than, the inner diameter 138 of the tube. The float 110, being generally rigid, can also provide support to the flexible tube wall 136. The seal formed between the support members 114 of the float and the wall 136 of the tube may be, but is not necessarily, a fluid-tight seal. As used herein, the term “seal” is also intended to encompass near-zero clearance or slight interference between the flanges 114 and the tube wall 136 providing a substantial seal, which is, in most cases, adequate for purposes of the disclosure.

The support members 114 are most preferably continuous ridges, in which case the sample may be centrifuged at lower speeds and slumping of the separated layers is inhibited. However, in alternative embodiments which are discussed further herein, the support members can be discontinuous or segmented bands having one or openings providing a fluid path in and out of the annular gap 150. The support members 114 may be separately formed and attached to the main body portion 112. Preferably, however, the support members 114 and the main body portion 112 form a unitary or integral structure.

In particular embodiments, the overall specific gravity of the separator float 110 should be between that of red blood cells (approximately 1.090) and that of plasma (approximately 1.028). In a preferred embodiment, the specific gravity is in the range of from about 1.089-1.029, more preferably from about 1.070 to about 1.040, and most preferably about 1.05. The overall specific gravity of the float 110 and the volume of the annular gap 150 may be selected so that the annular channel contains the buffy coat layers. The expanded buffy coat region can then be examined, e.g., under illumination and magnification, to identify circulating epithelial cancer or tumor cells or other target analytes.

The float may be formed of multiple materials having different specific gravities, so long as the overall specific gravity of the float is within the desired range. The overall specific gravity of the float 110 and the volume of the annular gap 150 may be selected so that some red cells and/or plasma may be retained within the annular gap, as well as the buffy coat layers. Upon centrifuging, the float 110 occupies the same axial position as the buffy coat layers and target cells and floats on the packed red cell layer. The buffy coat is retained in the narrow annular gap 150 between the float 110 and the inner wall of the tube 130. The expanded buffy coat region can then be examined, under illumination and magnification, to identify circulating epithelial cancer or tumor cells or other target analytes.

In one preferred embodiment, the density of the float 110 is selected to ride in the granulocyte layer of the blood sample. The granulocytes ride in, or just above, the packed red-cell layer and have a specific gravity of about 1.08-1.09. In this preferred embodiment, the specific gravity of the float is in this range of from about 1.08 to about 1.09 such that, upon centrifugation, the float rides in the granulocyte layer. The amount of granulocytes can vary from patient to patient by as much as a factor of about twenty. Therefore, selecting the float density such that the float rides in the granulocyte layer is especially advantageous since loss of any of the lymphocyte/monocyte layer, which rides just above the granulocyte layer, is avoided. During centrifugation, as the granulocyte layer increases in size, the float rides higher in the granulocytes and keeps the lymphocytes and monocytes at essentially the same position with respect to the float. Generally the cells of greatest interest are the “mononuclear cells,” which includes principally monocytes and lymphocytes, as well as other cells of interest, cancer cells and other epithelial cells. The “buffy coat” layer includes all the white cells, including all of the granulocytes, the platelets, and the other leukocytes that are not mononuclear cells.

The float 110 is formed of one or more generally rigid organic or inorganic materials, preferably a rigid plastic material, such as polystyrene, acrylonitrile butadiene styrene (ABS) copolymers, aromatic polycarbonates, aromatic polyesters, carboxymethylcellulose, ethyl cellulose, ethylene vinyl acetate copolymers, nylon, polyacetals, polyacetates, polyacrylonitrile and other nitrile resins, polyacrylonitrile-vinyl chloride copolymer, polyamides, aromatic polyamides (aramids), polyamide-imide, polyarylates, polyarylene oxides, polyarylene sulfides, polyarylsulfones, polybenzimidazole, polybutylene terephthalate, polycarbonates, polyester, polyester imides, polyether sulfones, polyetherimides, polyetherketones, polyetheretherketones, polyethylene terephthalate, polyimides, polymethacrylate, polyolefins (e.g., polyethylene, polypropylene), polyallomers, polyoxadiazole, polyparaxylene, polyphenylene oxides (PPO), modified PPOs, polystyrene, polysulfone, fluorine containing polymer such as polytetrafluoroethylene, polyurethane, polyvinyl acetate, polyvinyl alcohol, polyvinyl halides such as polyvinyl chloride, polyvinyl chloride-vinyl acetate copolymer, polyvinyl pyrrolidone, polyvinylidene chloride, specialty polymers, and so forth, and most preferably polystyrene, polycarbonate, polypropylene, acrylonitrite butadiene-styrene copolymer (“ABS”) and others.

In this regard, it is desirable to avoid the use of materials and/or additives that interfere with the detection or scanning method. For example, if fluorescence is utilized for detection purposes, the material utilized to construct the float 130 should not have interfering or “background” fluorescence at the wavelength of interest.

In some aspects, the compressibility and/or rigidity of the flexible tube and rigid float can be reversed. The float is flexible and designed to shrink in diameter at the higher pressures and moves freely within a rigid tube. The use of a compressible float allows for usage of transparent glass tubes which, in some instances, exhibit enhanced optical properties over polymeric tubes. Furthermore, this aspect generally reduces the tolerance requirements for the glass tubes (since the float would expand up against the tube wall after the pressure decreases), and a full range of float designs is possible.

A fluorescently labeled antibody, which is specific to the target epithelial cells or other analytes of interest, can be added to the blood sample in the assay test tube and incubated. In an exemplary embodiment, the epithelial cells are labeled with anti-EpCAM having a fluorescent tag attached to it. Anti-EpCAM binds to an epithelial cell-specific site that is not expected to be present in any other cell normally found in the blood stream. A stain or colorant, such as acridine orange, may also be added to the sample to cause the various cell types to assume differential coloration for ease of discerning the buffy coat layers under illumination and to highlight or clarify the morphology of epithelial cells during examination of the sample. Alternatively, as previously described above, the drug could be fluorescently labeled itself, so that the location of the drug could be determined during visual examination.

The separator float 110 is combined with the blood in the test tube for centrifugation. The float 110 may be introduced into the tube 130 after the blood sample is introduced into the sample tube 130 or otherwise may be placed therein beforehand. The tube and float assembly 100 containing the sample is then centrifuged. Operations required for centrifuging the blood by means of the subject tube/float system 100 are not expressly different from the conventional case, although, as stated above, reduced centrifuge speeds may be possible and problems of slumping may be reduced. An adaptor may optionally be utilized in the rotor to prevent failure of the flexible tube due to stress.

During centrifugation, the sample tube is spun at a rotational speed sufficient to cause several effects. In particular, the resultant hydrostatic pressure deforms or flexes the wall 136 so as to enlarge the diameter of the tube from a first cross-sectional inner diameter to a second diameter, the second diameter being greater than the first diameter. The second diameter is sufficiently large to permit the blood components and the float 110 to move axially under centrifugal force within the tube 130. The blood sample is separated into six discrete and distinct layers according to density, which are, from bottom to top (most dense to least dense): packed red blood cells, reticulocytes, granulocytes, lymphocytes/monocytes, platelets, and plasma. The epithelial cells sought to be imaged tend to collect by density in the buffy coat layers, i.e., in the granulocyte, lymphocyte/monocyte, and platelet layers. Due to the density of the float, the float occupies the same axial position within the sample tube as the buffy coat layers/constituents which thus occupy the narrow annular volume 150, potentially along with a small amount of the red cell and/or plasma). Put another way, the float moves into alignment with at least the buffy coat constituents of the blood sample.

After centrifugal separation is complete and the centrifugal force is removed, the tube 130 returns to its original diameter to capture or retain the float and the buffy coat layers and target analytes within the annular volume 150. The tube/float system can be transferred to a microscope or optical reader to identify any target analytes in the blood sample. Depending on the subsequent use of the float, the annular volume may be considered to make up one or more analysis areas.

Centrifugation may not be required. Sometimes the application of pressure alone to the inside of the tube, or simply the expansion of the tube (or the compression of the float) is required. For example, such pressure can be produced through the use of a vacuum source on the outside of the tube. Such an application also allows for the top of the sample tube to be kept open and easily accessible. Additionally, the use of a vacuum source may be easier to implement in some situations than the application of a centrifugal force. Additionally, any method of tubular expansion/contraction (or float compression) such as mechanical, electrical, magnetic, etc., can be implemented. Once the tube is expanded (or the float is compressed), the float will move to the proper location due to buoyancy forces created by the density variations within the sample.

In additional embodiments described herein, a removal device, such as a syringe, is then used to extract the buffy coat layers/constituents from the annular volume. The intent here is to extract the target cells of interest, so it is acceptable to remove some of the red blood cells and/or plasma during this process as well. If tags have not yet been added, they may be added now to tag or label the “target” cells of interest. Again, the tags are any kind that an analytical instrument or detector could detect, e.g. fluorescent, radioactive, etc. The tags may be in the removal device itself, or they can be added separately.

The sample is then applied, such as by being “squirted”, through the instrument/detector and the tagged cells are analyzed. It may be sufficient to count the number of tagged cells. However, in further embodiments, the ‘positive’ sample cells are diverted into a holder for further analysis. Means of separating such cells are known in the art and can be similar to those used in flow cytometry, for example by coordinating the timing of the instrument/detector with the holder. The positive sample can then be further analyzed, for example by preparing a slide for further examination. This ‘squirt-n-divert’ method results in a smaller sample volume that is easier to analyze compared to the original blood sample, which was many times larger.

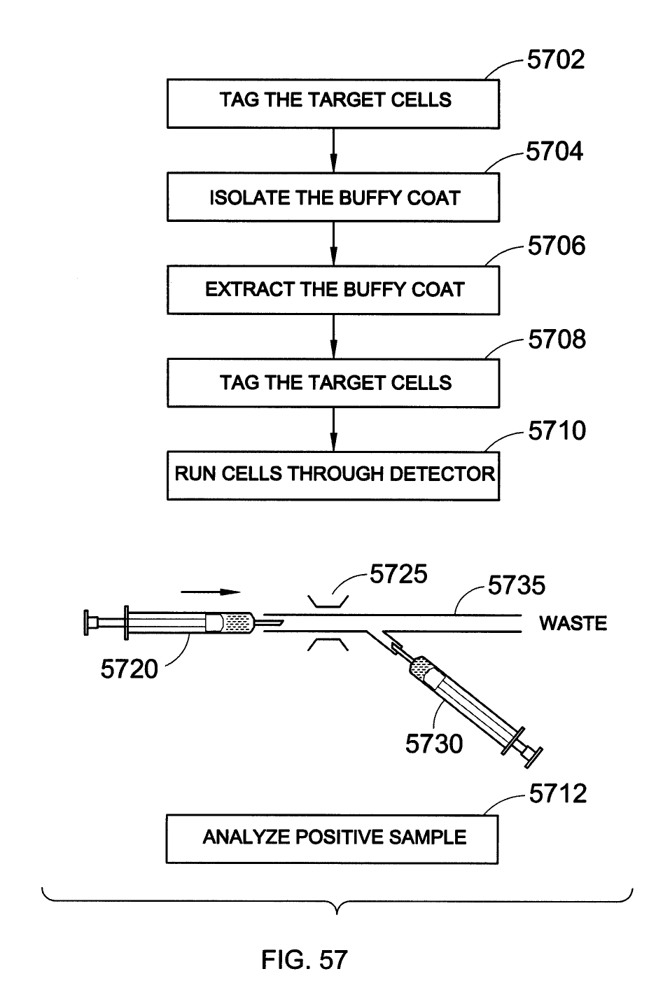

FIG. 57 is a diagram illustrating some of the general methods described above. In step 5702, the target cells in the buffy coat layers of the blood sample can be tagged prior to centrifugation. In step 5704, the buffy coat is isolated, e.g. by centrifugation. In step 5706, the sample containing the buffy coat, and reduced in volume compared to the original blood sample, is extracted from the sample tube. In step 5708, if the target cells were not already tagged, they can be tagged now. Alternatively, they can be tagged using different tags suitable for use with the given instrument/detector. In step 5710, the reduced volume is run through the detector. As illustrated here, the reduced volume with the tagged target cells begin in syringe 5720 and are injected into detector 5725 which separates the ‘positive’ sample (i.e. target cells) and diverts them into holder 5730. The ‘negative’ sample goes to waste 5735, i.e. is disposed of. Finally, in step 5712, the positive sample is further analyzed.

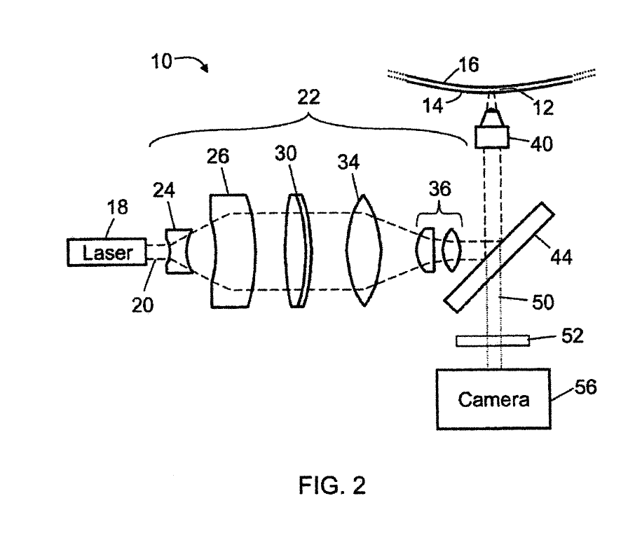

The separator float/tube system of FIG. 1 can be used to isolate the cancer cells or other cells of interest in the assay test tube and the control test tube. FIG. 2 shows a diagnostic system which can be used to visually examine the test tubes and determine the effect of the drug on one or more given cell types in the blood sample. This diagnostic system is described in more detail in U.S. Pat. No. 7,397,601, the entirety of which is hereby incorporated by reference herein.

Referring to FIG. 2, a microscope system 10 images a microscope field of view coinciding with a buffy coat sample disposed in a generally planar portion of an annular gap 12 between a light-transmissive test tube wall 14 and a float wall 16 of a float disposed in the test tube. The microscope field of view is generally planar in spite of the curvatures of the test tube and the float, because the microscope field of view is typically much smaller in size than the radii of curvature of the test tube wall 14 and the float wall 16. Although the field of view is substantially planar, the buffy coat sample disposed between the light-transmissive test tube wall 14 and the float wall 16 may have a thickness that is substantially greater than the depth of view of the microscope system 10. The test tube is mounted in fixed position respective to the microscope system 10 in a manner conducive to scanning the microscope field of view across the annular gap. As will be discussed, suitable mechanisms are preferably provided for effectuating relative rotational and/or translational scanning of the field of view over the annular gap containing the buffy coat sample.

The microscope system 10 may include a laser 18, such as a gas laser, a solid state laser, a semiconductor laser diode, or so forth, that generates source light 20 (indicated in FIG. 2 by dashed lines) in the form of a laser beam having an illumination wavelength and a non-uniform spatial distribution that is typically Gaussian or approximately Gaussian in shape with a highest intensity in a central region of the beam and reduced intensity with increasing distance from the beam center. An optical train 22 is configured to receive the spatially non-uniform source light 20 and to output a corrected spatial distribution.

A beam spreader includes a concave lens 24 that generally diverges the laser beam, and a collimating lens 26 that collimates the spread beam at a larger diameter that substantially matches the diameter of a Gaussian spatial characteristic of a beam homogenizer 30. The beam homogenizer 30 flattens the expanded laser beam by substantially homogenizing the Gaussian or other non-uniform distribution of the source light to produce output light having improved spatial uniformity. Alternatively, a stationary diffuser can be used as component 30. The diffuser may, for example, be a holographic diffuser. Such holographic diffusers employ a hologram providing randomizing non-periodic optical structures that diffuse the light to impart improved spatial uniformity. However, the diffusion of the light also imparts some concomitant beam divergence. Typically, stronger diffusion of the light tend to impart more spatial uniformity, but also tends to produce greater beam divergence. Holographic diffusers are suitably classified according to the full-width-at-half-maximum (FWHM) of the divergence angle, with larger divergence angles typically providing more diffusion and greater light uniformity, but also leading to increased light loss in the microscope system due to increased beam divergence.

A focusing lens 34 and cooperating lenses 36 reduce the expanded and flattened or homogenized laser beam down to a desired beam diameter for input to an objective 40 that is focused on the microscope field of view. A dichroic mirror 44 is selected to substantially reflect light at the wavelength or wavelength range of the laser beam, and to substantially transmit light at the fluorescence wavelength or wavelength range of the fluorescent dye used to tag rare cells in the buffy coat sample.

The optical train 22 including the stationary optical components 24, 26, 30, 34, 36 is configured to output a corrected spatial distribution to the objective 40 that when focused by the objective 40 at the microscope field of view provides substantially uniform static illumination over substantially the entire microscope field of view. The objective 40 focuses the corrected illumination onto the microscope field of view. The objective 40 may include a single objective lens, or may include two or more objective lenses. The focus depth of the microscope system 10 is adjustable, for example by adjusting a distance between the objective 40 and the light-transmissive test tube wall 14. Additionally or alternatively, the focus depth may be adjusted by relatively moving two or more lenses or lensing elements within the objective 40.

The beam homogenizer 30 is designed to output a substantially uniform homogenized beam for a Gaussian input beam of the correct diameter. However, the objective 40 typically introduces some spatial non-uniformity. Accordingly, one or more of the stationary optical components, such as the spreading lens 24, collimating lens 26, focusing lens 34, and/or focusing lenses 36 are optionally configured to introduce spatial non-uniformity into the spatial distribution such that the beam when focused by the objective 40 provides substantially uniform static illumination of the microscope field of view. In some contemplated embodiments, this corrective spatial non-uniformity is introduced by one or more dedicated optical components (not shown) that are included in the optical train 22 for that purpose.

The substantially uniform static illumination of the microscope field of view can be used for fluorescence of any fluorescent dye-tagged epithelial cells disposed within the microscope field of view. Additionally, the fluorescent dye typically imparts a lower-intensity background fluorescence to the buffy coat. The fluorescence is captured by the objective 40, and the captured fluorescence 50 (indicated in FIG. 2 by dotted lines) passes through the dichroic mirror 44, and through an optional filter 52 for removing any stray source light, to be imaged by a camera system 56. The camera system 56 may, for example, include a charge coupled device (CCD) camera for acquiring electronic images that can be stored in a computer, memory card, or other non-volatile memory for subsequent image processing.

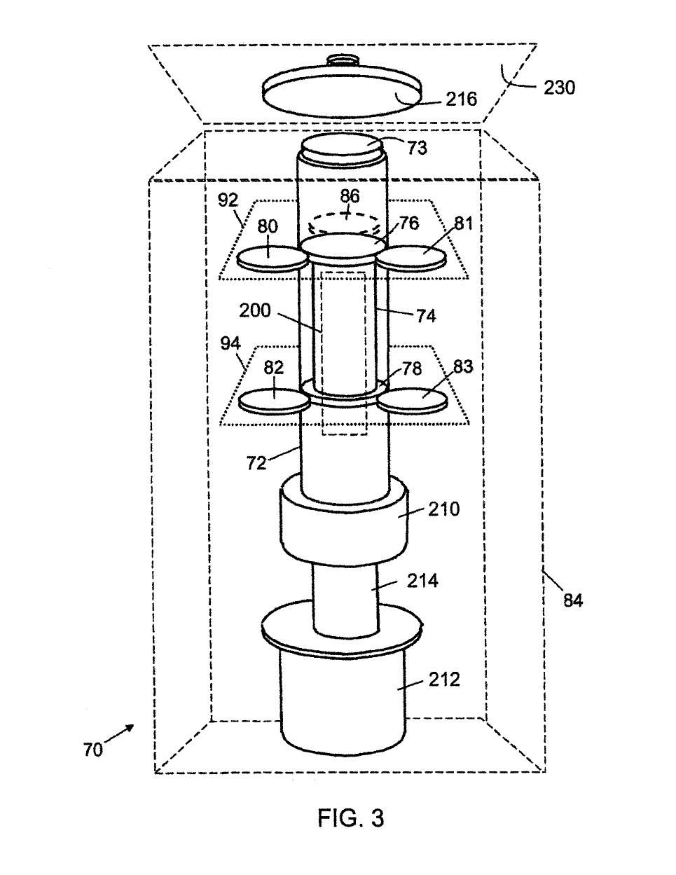

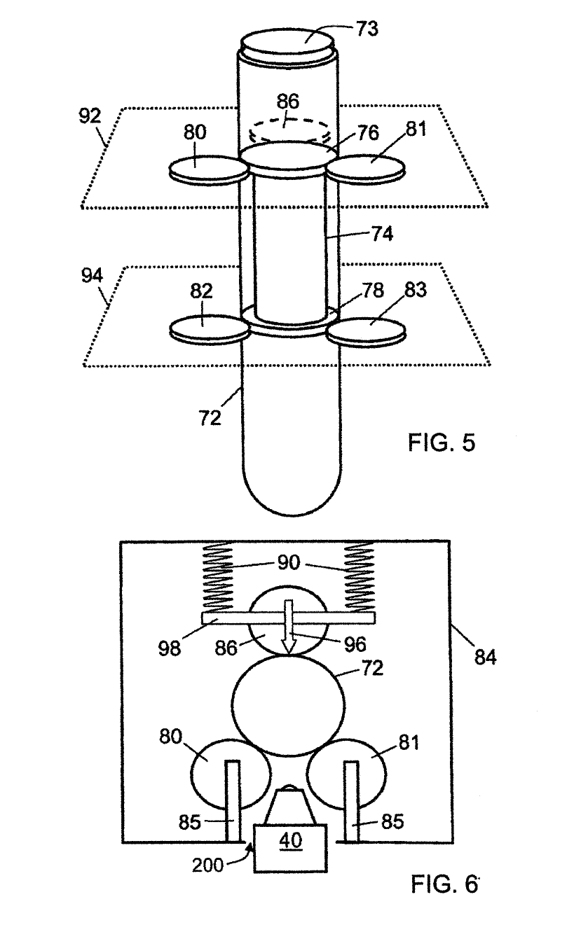

FIGS. 3-6 illustrate a test tube holder to be used with the light system of FIG. 2. A test tube holder 70 has mounted therein a test tube 72 that is sealed by a test tube stopper 73. The sealed test tube 72 contains a float 74 and blood that has been suitably processed and centrifuged to separate out components including red blood cells, plasma, and a buffy coat, as previously described. After centrifuging the float 74 is disposed along the test tube axis 75 (drawn and labeled in FIG. 6). The buffy coat layer may be generally disposed in the annular gap 12 between the test tube wall 14 and the float wall 16. Annular sealing ridges 76, 78 at ends of the float 74 engage an inside surface of the test tube 72 when the test tube is at rest so as to seal the annular gap 12. During centrifuging, however, the test tube 72 expands to provide fluid communication across the ridges 76, 78 so as to enable the buffy coat to substantially collect in the annular gap 12.

At least one first alignment bearing, namely two radially spaced apart first alignment bearings 80, 81 in the example test tube holder 70, are disposed on a first side of the annular sampling region 12. At least one second alignment bearing, namely two second radially spaced apart alignment bearings 82, 83 in the example test tube holder 70, are disposed on a second side of the annular sampling region 12 opposite the first side of the annular sampling region 12 along the test tube axis 75. The alignment bearings 80, 81, 82, 83 are fixed roller bearings fixed to a housing 84 by fastening members 85 (shown only in FIG. 6).

At least one biasing bearing, namely two biasing bearings 86, 87 in the example test tube holder 70, are radially spaced apart from the alignment bearings 80, 81, 82, 83 and are spring biased by springs 90 to press the test tube 72 against the alignment bearings 80, 81, 82, 83 so as to align a side of the annular sampling region 12 proximate to the objective 40 respective to the alignment bearings 80, 81, 82, 83. In the example test tube holder 70, the two first alignment bearings 80, 81 and the first biasing bearing 86 are radially spaced apart by 120° intervals and lie in a first common plane 92 on the first side of the annular sampling region 12. Similarly, the two second alignment bearings 82, 83 and the second biasing bearing 87 are radially spaced apart by 120° intervals and lie in a second common plane 94 on the second side of the annular sampling region 12. The springs 90 are anchored to the housing 84 and connect with the biasing bearings 86, 87 by members 98.

More generally, the bearings 80, 81, 86 and the bearings 82, 83, 87 may have radial spacings other than 120°. For example the biasing bearing 86 may be spaced an equal radial angle away from each of the alignment bearings 80, 81. As a specific example, the biasing bearing 86 may be spaced 135° away from each of the alignment bearings 80, 81, and the two alignment bearings 80, 81 are in this specific example spaced apart by 90°.

Optionally, the first common plane 92 also contains the float ridge 76 so that the bearings 80, 81, 86 press against the test tube 72 at the ridge 76, and similarly the second common plane 94 optionally also contains the float ridge 78 so that the bearings 82, 83, 87 press against the test tube 72 at the ridge 78. This approach reduces a likelihood of distorting the annular sample region 12. The biasing bearings 86, 87 provide a biasing force 96 that biases the test tube 72 against the alignment bearings 80, 81, 82, 83.

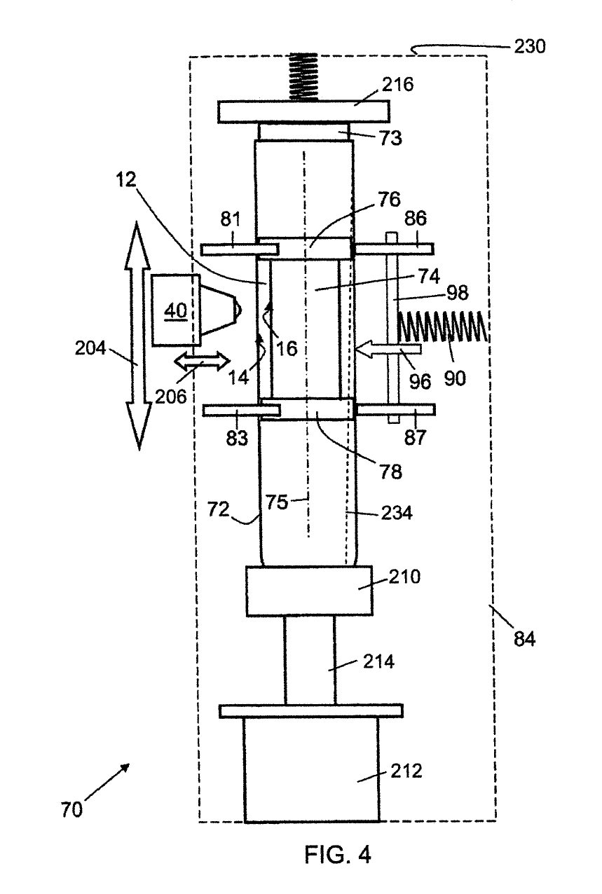

The housing includes a viewing window 200 that is elongated along the tube axis 75. The objective 40 views the side of the annular sample region 12 proximate to the objective 40 through the viewing window 100. In some embodiments, the objective 40 is linearly translatable along the test tube axis 75 as indicated by translation range double-arrow indicator 204. This can be accomplished, for example, by mounting the objective 40 and the optical train 22′ on a common board that is translatable respective to the test tube holder 70. In another approach, the microscope system 10 is stationary, and the tube holder 70 including the housing 84 is translated as a unit to relatively translate the objective 40 across the window 100. In yet other embodiments, the objective 40 translates while the optical train 22 remains stationary, and suitable beam-steering components (not shown) are provided to input the beam to the objective 40. The objective 40 is also focusable, for example by moving the objective 40 toward or away from the test tube 72 over a focusing range 206 (translation range 204 and focusing range 206 indicated only in FIG. 4).

Scanning of the annular sampling region 12 calls for both translation along the test tube axis, and rotation of the test tube 72 about the test tube axis 75. To achieve rotation, a rotational coupling 210 is configured to drive rotation of the test tube 72 about the tube axis 75 responsive to a torque selectively applied by a motor 212 connected with the rotational coupling 210 by a shaft 214. The rotational coupling 210 of the example test tube holder 70 connects with the test tube 72 at an end or base thereof. At an opposite end of the test tube 72, a spring-loaded cap 216 presses against the stopper 73 of the test tube 72 to prevent the rotation from causing concomitant translational slippage of the test tube 72 along the test tube axis 75.

In order to install the test tube 72 in the test tube holder 70, the housing 84 is provided with a hinged lid or door 230 (shown open in FIG. 3 and closed in FIG. 4). When the hinged lid or door 230 is opened, the spring-loaded cap 216 is lifted off of the stopper 73 of the test tube 72. Optionally, the support members 98 that support the biasing bearings 86, 87 include a manual handle or lever (not shown) for manually drawing the biasing bearings 86, 87 away from the test tube 72 against the biasing force of the springs 90 so as to facilitate loading or unloading the test tube 72 from the holder 70.

The test tube holder 70 advantageously can align the illustrated test tube 72 which has straight sides. The test tube holder 70 can also accommodate and align a slightly tapered test tube. The held position of a tapered test tube is indicated in FIG. 4 by a dashed line 234 which indicates the tapered edge of a tapered test tube. The illustrated tapering 234 causes the end of the test tube closest to the rotational coupling 210 to be smaller diameter than the end of the test tube closest to the spring-loaded cap 216. The biasing of the biasing bearings 86, 87 presses the test tube against the alignment bearings 81, 82, 83, 84 to maintain alignment of the portion of the annular sample region 12 proximate to the objective 40 in spite of the tapering 234. It will be appreciated that the holder 70 can similarly accommodate and align a test tube having an opposite taper in which the end closes to the rotational coupling 210 is larger in diameter than the end closest to the spring-loaded cap 216.

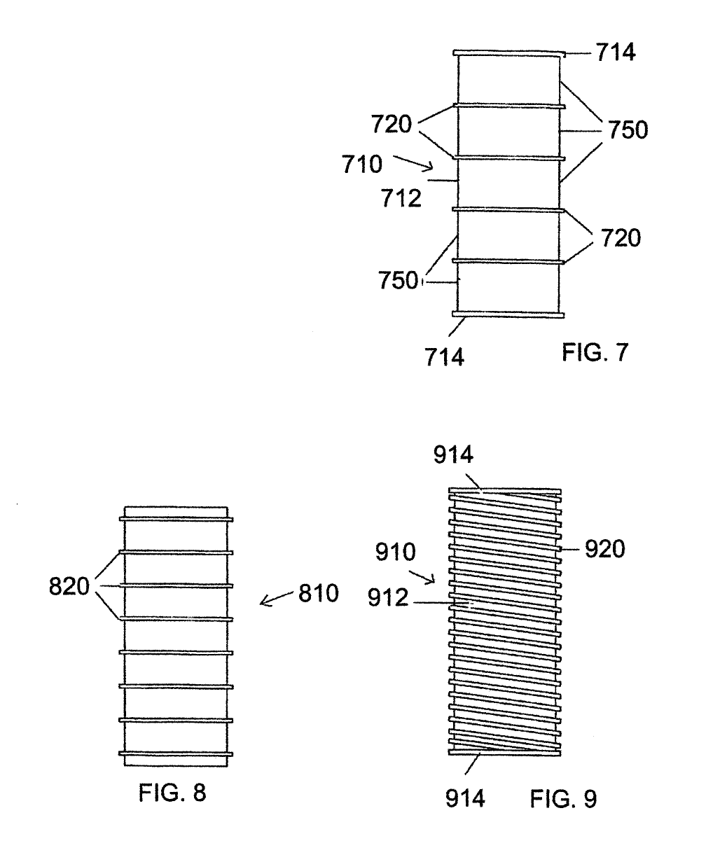

FIGS. 7-22 show various other embodiments of a separator float which can be used in practicing the methods of the present disclosure. These embodiments are also seen in U.S. Pat. No. 7,074,577, the entirety of which is fully incorporated by reference herein.

FIG. 7 illustrates a float 710 according to a further embodiment. The float 710 has a plurality of ribs 720 axially spaced along a central body portion 712, and plural annular channels 750 are defined therebetween. Optional sealing ridges 714 are disposed at opposite ends of the float. Again, the illustrated embodiment depicts continuous ribs, however, it will be recognized that the support ribs may likewise be broken or segmented to provide an enhanced flow path between adjacent annular channels 750.

FIG. 8 illustrates a further float embodiment 810, similar to the embodiment of FIG. 7, the above descriptions of which are equally applicable thereto. However, the float 810 differs in that it lacks sealing ridges at the opposite ends thereof, which may optionally be provided, and the spacing between the ribs 820 is different as well.

FIG. 9 illustrates a further float embodiment 910, wherein a helical support member or ridge 920 is provided. That is, instead of discrete annular bands, multiple turns of the helical ridge 920 provides a series of spaced apart ridges on the main body portion 912, which defines a corresponding helical channel. The helical ridge 920 is illustrated as continuous, however, the helical band may instead be segmented or broken into two or more segments, e.g., to provide path for fluid flow between adjacent turns of the helical buffy coat retention channel. Optional sealing ridges 914 appear at each axial end of the float 910.

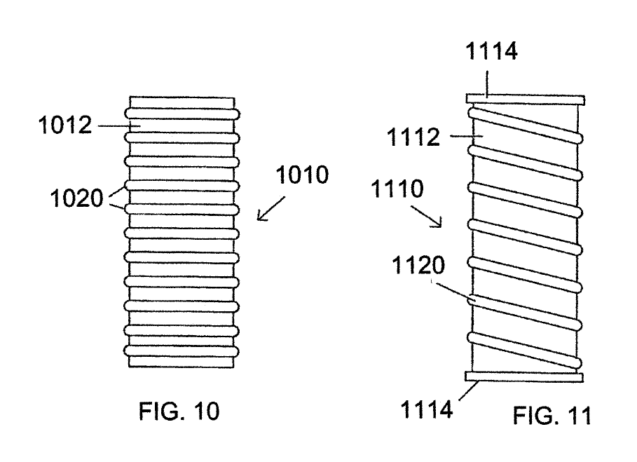

FIG. 10 illustrates another ribbed embodiment 1010. Radial support members 1020 extend radially from the main body portion 1012. The support members 1020 each have a generally curved or rounded cross-sectional profile. Again, the support members 1020 are shown as continuous but may, in alternative embodiments, be discontinuous or segmented. End sealing ridges are not present in FIG. 10, but may optionally be provided.

FIG. 11 illustrates another embodiment of a separator float 1110. Here, the support member 1120 is helical, and extends from main body portion 1112. End sealing ridges 1114 are present, though again they are optional.

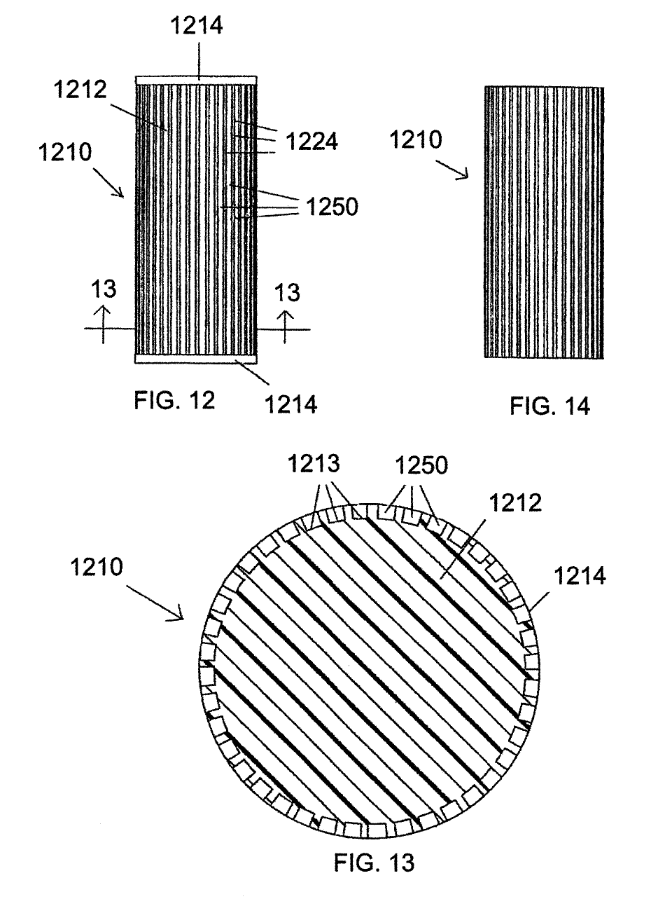

Referring now to FIG. 12 and FIG. 13, there is shown a splined separator float 1210. The float 1210 includes a plurality of axially-oriented splines or ridges 1224 radially spaced about a central body portion 1212. Optional end sealing ridges 1214 are disposed at opposite ends of the float. The splines 1224 and the optional end sealing ridges 1214 protrude from the main body 1212 to engage and provide support for the deformable tube. Where provided, the end sealing ridges 1214 provide a sealing function as described above. The axial protrusions 1224 define fluid retention channels 1250, between the tube inner wall and the main body portion 1212. The surfaces 1213 of the main body portion disposed between the protrusions 1224 may be curved, e.g., when the main body portion is cylindrical, however, flat surfaces 1213 are also contemplated. Although the illustrated embodiment depicts splines 1224 that are continuous along the entire axial length of the float, segmented or discontinuous splines are also contemplated.

FIG. 14 illustrates an embodiment of the float 1210 wherein the end sealing ridges are not provided.

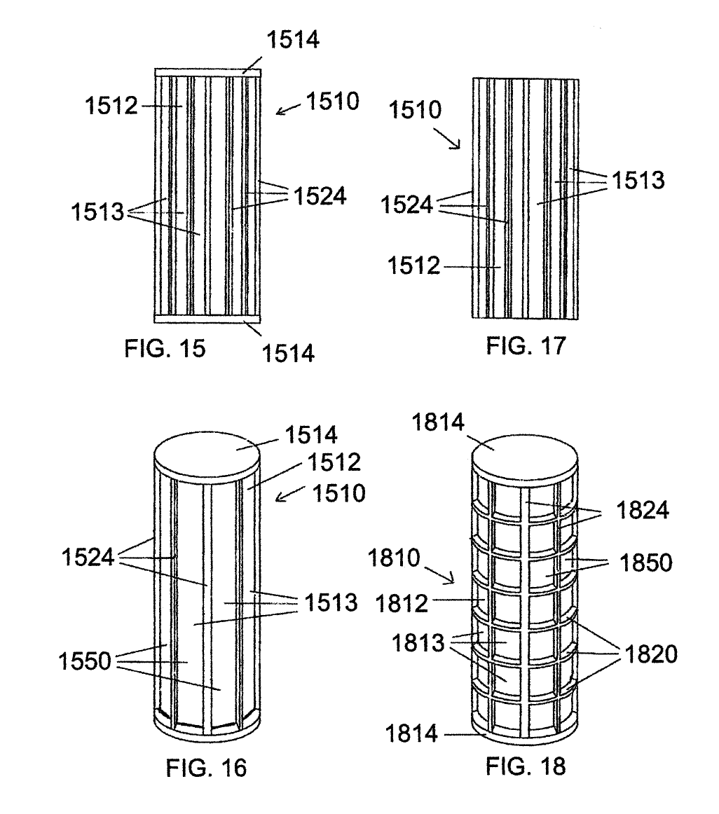

FIG. 15 is a side view of another embodiment of the float 1510, while FIG. 16 is a perspective view of the float. Here, axially aligned ribs or splines 1524 protrude from the main body portion 1512. The float 1510 includes optional end sealing ridges 1514 which are radially aligned and are disposed at opposite ends of the float 1510. Fluid retention channels 1550 formed between adjacent splines 1524 are defined by adjacent splines 1524 and surfaces 1513 on the main body portion 1512. The surfaces 1513 are depicted as generally flat, although curved surfaces are also contemplated. The axial splines 1524 are continuous along the length of the tube; however, segmented or discontinuous splines are also contemplated.

FIG. 17 illustrates an embodiment of the float 1510 wherein the end sealing ridges are not provided.

Referring now to FIG. 18, there is shown yet another embodiment 1810. The support members. The support means 1820 can be described as an intersecting network of annular rings or ribs 1826 and axial splines 1824. Optional end sealing ridges 1814 are disposed at opposite ends of the float. The support members 1820 and the optional sealing ridges 1814 engage and provide support for the deformable tube. Where provided, the end sealing ridges 1814 provide a sealing function as described above. The raised support members 1820 define a plurality of fluid retention windows 1850 formed between the tube inner wall and the main body portion 1812. Surfaces 1813 of the main body portion 1812 corresponding to the windows 1850 may be curved, e.g., when the main body portion is cylindrical, however, flat surfaces are also contemplated. Although the illustrated embodiment depicts the support members 1820 as a network of annular ribs and axial splines which is continuous, breaks may also be includes in the annular and/or axial portions of the network 1820, e.g., to provide a fluid path between two or more of the windows 1850.

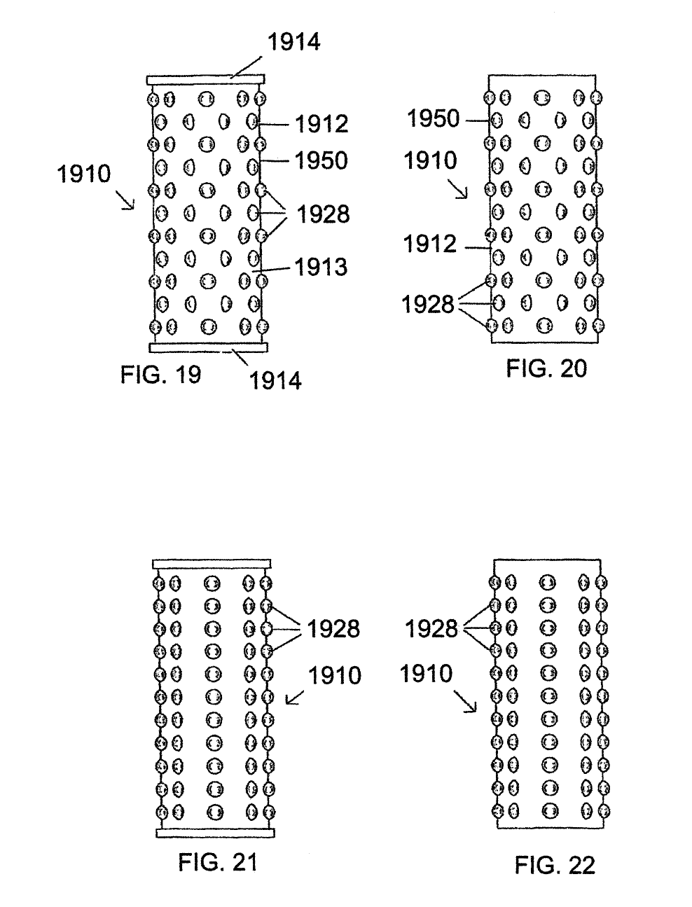

FIGS. 19-22 illustrate several floats having a plurality of protrusions thereon for providing support for the deformable walls of the sample tube.

Referring to FIG. 19, the float 1910 includes multiple rounded protrusions 1928 spaced over the surface 1913 of the central body portion 1912 in a staggered pattern. Optional end sealing ridges 1914 are disposed at opposite ends of the float 1910. The protrusions 1928 and the optional end sealing ridges 1914 radially protrude from the main body 1912 and traverse an annular gap 1950 to engage and provide support for the deformable tube wall. When provided, the end sealing ridges 1914 provide a sealing function as described above. The surface 1913 of the main body portion disposed between the protrusions may be curved, e.g., when the main body portion is cylindrical, or, alternatively, may have flat portions or facets.

FIG. 20 illustrates an embodiment of the float 1910 wherein the end sealing ridges are not provided.

In FIG. 21, the protrusions 1928 are spaced over the surface in an aligned pattern. End sealing ridges 1914 are provided.

In FIG. 22, the protrusions 1928 are spaced over the surface in an aligned pattern, and end sealing ridges are absent.

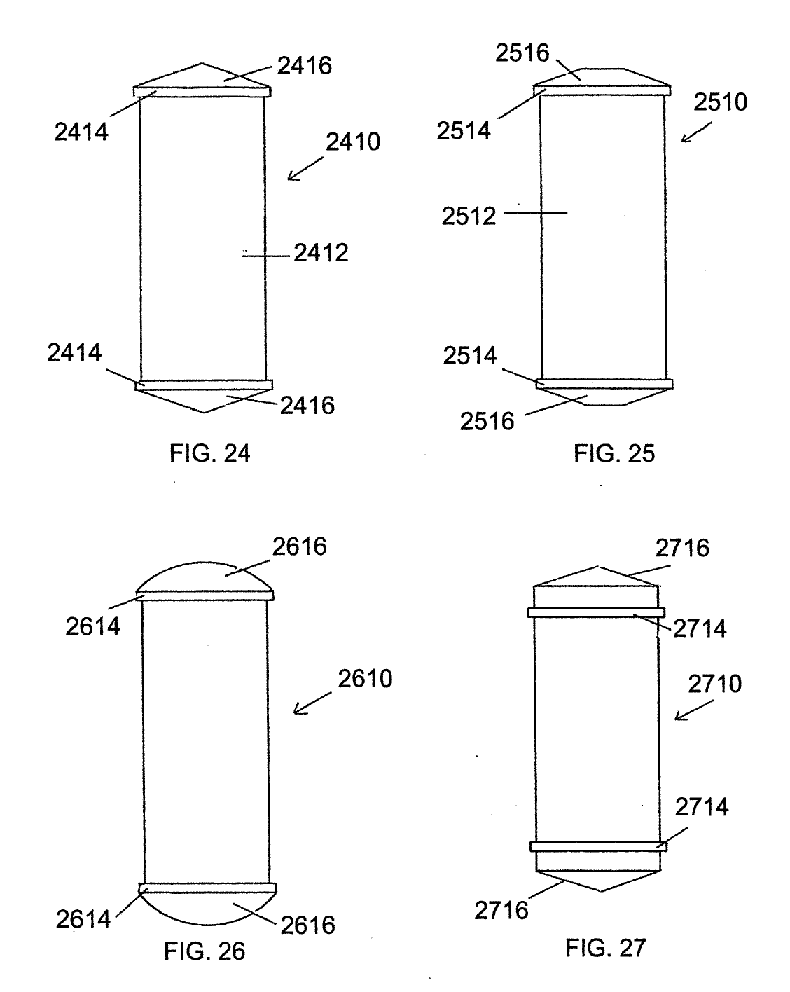

Additional embodiments of separator floats are shown in FIGS. 24-29. These embodiments are also seen in U.S. Pat. No. 7,220,593, the entirety of which is fully incorporated by reference herein.

FIG. 24 illustrates a float 2410 that includes a main body portion 2412 and sealing rings 2414. The ends of the main body portion may be considered as including a tapered or cone-shaped endcap member 2416 disposed at each end. The tapered endcaps 2416 are provided to facilitate and direct the flow of cells past the float 2410 and sealing ridges 2414 during centrifugation.

FIG. 25 illustrates a float 2510 that includes a main body portion 2512 and sealing ridges 2514 similar to FIG. 24. Here, the endcap members 2516, disposed at each end, have a frustoconical shape.

FIG. 26 illustrates a float 2610 having generally convex or dome-shaped endcap members 2616, which cap the sealing ridges 2614. The endcaps 2616 may be hemispherical, hemiellipsoidal, or otherwise similarly sloped, are provided. Again, the sloping ends 2616 are provided to facilitate density-motivated cell and float movement during centrifugation.

The geometrical configurations of the endcap units 2416, 2516, and 2616 illustrated in FIGS. 24-26, respectively, are intended to be exemplary and illustrative only, and many other geometrical shapes (including concave or convex configurations) providing a curved, sloping, and/or tapered surface around which the blood sample may flow during centrifugation. Additional exemplary shapes contemplated include, but are not limited to tectiform and truncated tectiform; three, four, or more sided pyramidal and truncated pyramidal, ogival or truncated ogival; geodesic shapes, and the like.

FIG. 27 illustrates a float 2710 wherein the sealing ridges are 2714 are axially displaced from the ends. Optional endcap members 2716 appear as conical in the illustrated embodiment. However, it will be recognized that the endcaps 2716, if present, any other geometrical configuration which provides a sloped or tapered surface may be used, as described above.

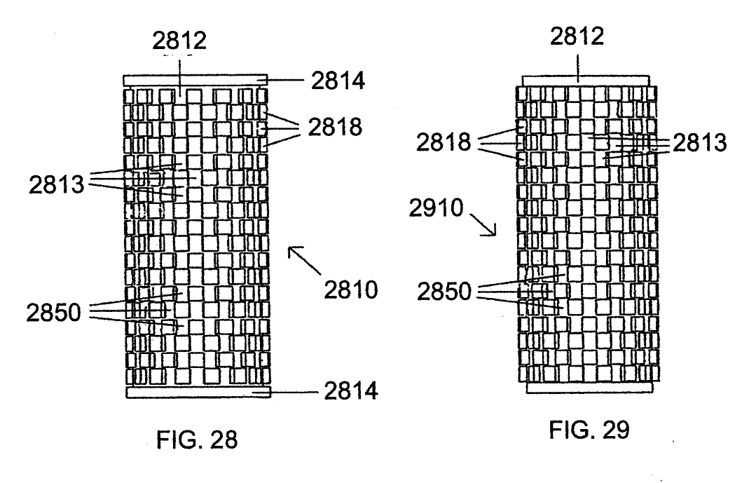

FIG. 28 and FIG. 29 illustrate float embodiments 2810 and 2910, respectively, which include multiple raised facets 2828 spaced over the surface of a main or central body portion 2812. Optional end sealing ridges 2814 are present in FIG. 28, but not FIG. 29. The facets 2828 and the optional end sealing ridges 2814 radially protrude from the main body 2812 and traverse an annular gap to engage and provide support for the test tube sidewall and define a plurality of fluid retention windows 2850. Where provided, the end sealing ridges 2814 provide a sealing function as described above. The surfaces 2813 of the main body portion, disposed between the protrusions 2828 and forming a surface defining the fluid-retention windows 2850, may be curved surfaces, e.g., when the main body portion is cylindrical. Alternatively, the surfaces 2813 may be flat. In alternative embodiments, the size, spacing density, and alignment patterns of the facets 2818 can be modified extensively.

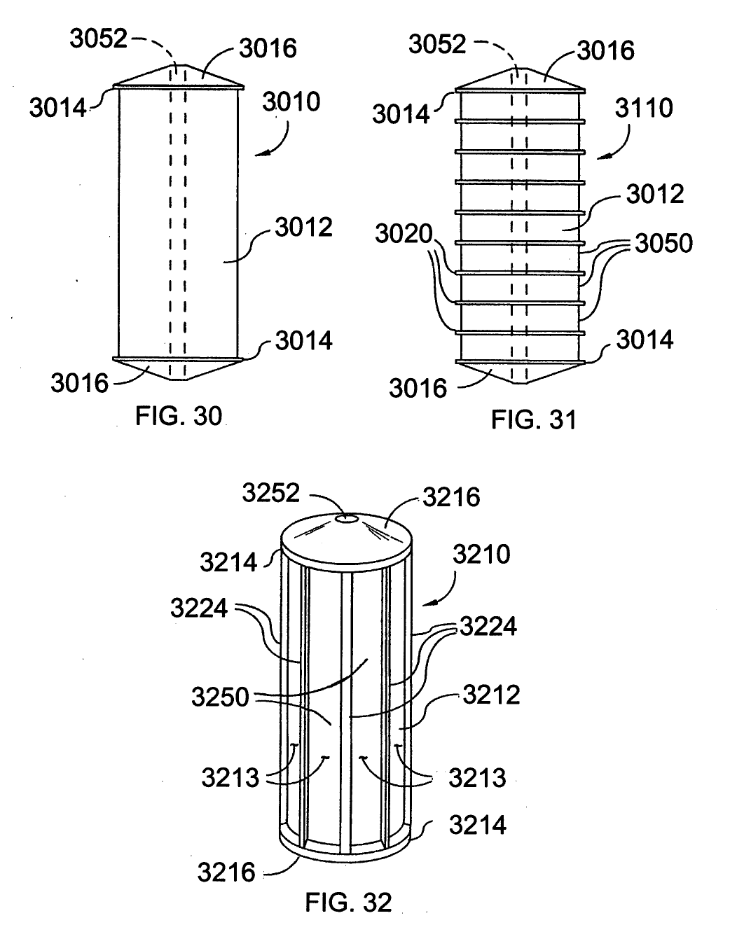

FIG. 30 and FIG. 31 illustrate float embodiments 3010 and 3110, respectively. The main body portion 3012 has a diameter that is smaller than the inner diameter of the test tube. Optionally tapered ends 3016 are provided to facilitate and direct the flow of cells past the float 3010 and sealing ridges 3014 during centrifugation. A central bore 3052, shown in broken lines, provides a pressure relief outlet to alleviate any pressure build up in the lower fluid layers due to the contraction of the tube walls. FIG. 31 includes radially extending ribs 3020 spaced along the axial direction of the main body portion between the two ends of the float. Multiple annular channels 3050 are defined between the main body portion 3012 and the inner tube wall. Although the illustrated embodiment depicts continuous ribs, it will be recognized that the support ribs may likewise be broken or segmented to provide an enhanced flow path between adjacent annular channels 3050.

FIG. 32 shows a splined separator float 3210, including a plurality of axially oriented splines or ridges 3224 which are radially spaced about a central body portion 3212. End sealing ridges 3214 and optionally tapered ends 3216 are provided to facilitate and direct the flow of cells past the float 3210 and sealing ridges 3214 during centrifugation. The splines 3224 and the end sealing ridges 3214 protrude from the main body 3212 to engage and provide support for the deformable tube once centrifugation is completed. The axial protrusions 3224 define fluid retention channels 3250, between the tube inner wall and the main body portion 3212. The surfaces 3213 of the main body portion disposed between the protrusions 3224 may be curved, e.g., when the main body portion 3212 is cylindrical, however, flat surfaces 3213 are also contemplated. Although the illustrated embodiment depicts splines 3224 that are continuous along the entire axial length of the float 3210, segmented or discontinuous splines are also contemplated. A pressure relief bore 3252 extends axially and centrally through the float 3210. In other embodiments, one or more of such pressure relief bores, of similar or different shape, can be included in the main body of the float.

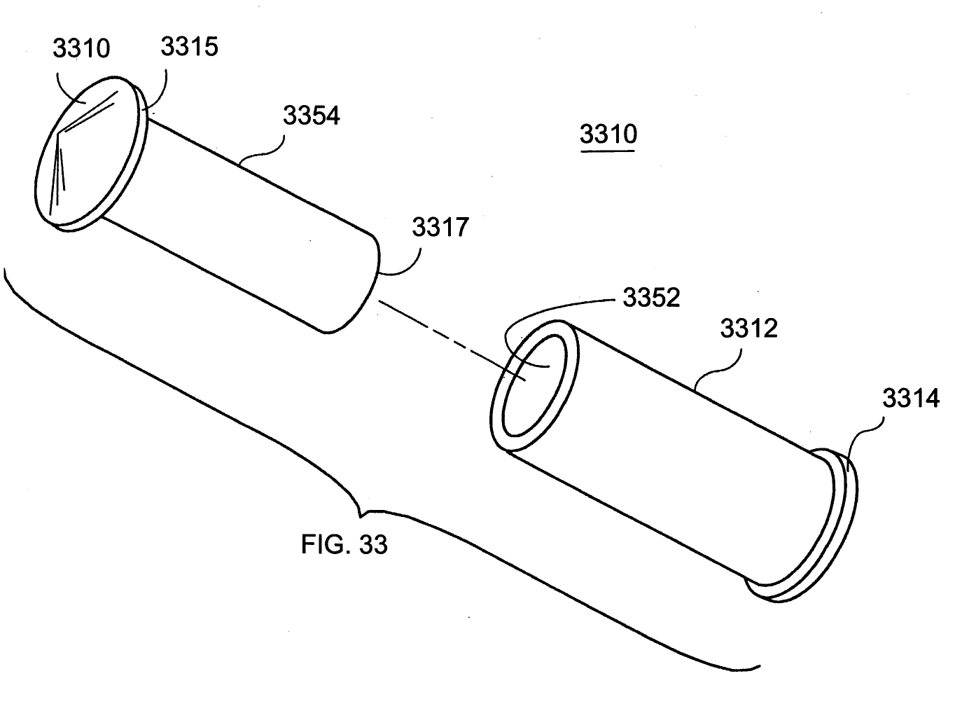

FIG. 33 illustrates a two-piece float 3310 in accordance with a preferred embodiment of the present disclosure, shown in exploded view. A first, main body portion or sleeve 3312 includes a central bore 3352, which is sized to slidably receive a second, piston-like center portion 3354. The outer body member 3312 includes a flange or sealing ring 3314, which is at its lower or bottom end. A sealing ridge or flange 3315 is disposed at the upper end of the piston section 3354 during operation. Optionally tapered ends 3317 are preferably provided at the upper and lower (during operation) ends of the piston portion 3354 to facilitate and direct the flow of cells past the sealing ridges 3314 and 3315 during centrifugation.

In operation, the piston portion 3354 is fully received within the central bore 3352 of the main body member 3312. As stated above, the float 3310 is oriented in the tube so that the sealing ridge 3315 is at the top and the sealing ridge 3314 is toward the bottom of the tube. The two portions may be formed of the same material or different materials, so long as the overall specific gravity of the float 3310 is in a suitable range for buffy coat capture. In an especially preferred embodiment, the central piston portion 3354 is formed of a slightly higher specific gravity material than the outer portion 3312, which insures that the two portions stay together during centrifugation. Alternatively, the two float members are formed of the same material and/or a frictional fit sufficient to keep the float members together during centrifugation is provided.

As the tube containing the blood sample and float 3310 is centrifuged, the two pieces 3312 and 3354 stay together and act in the same manner as a one-piece float to axially expand the buffy coat layers. When separation and layering of the blood components is complete and centrifugation is slowed, pressure may build in the red blood cell fraction trapped below the float, e.g., where contraction of the tube continues after initial capture of the float by the tube wall. Any such pressure in the trapped red blood cell region forces the center piece 3354 upward, thus relieving the pressure, and thereby preventing the red blood cells from breeching the seal between the sealing rings 3314 and the tube wall.

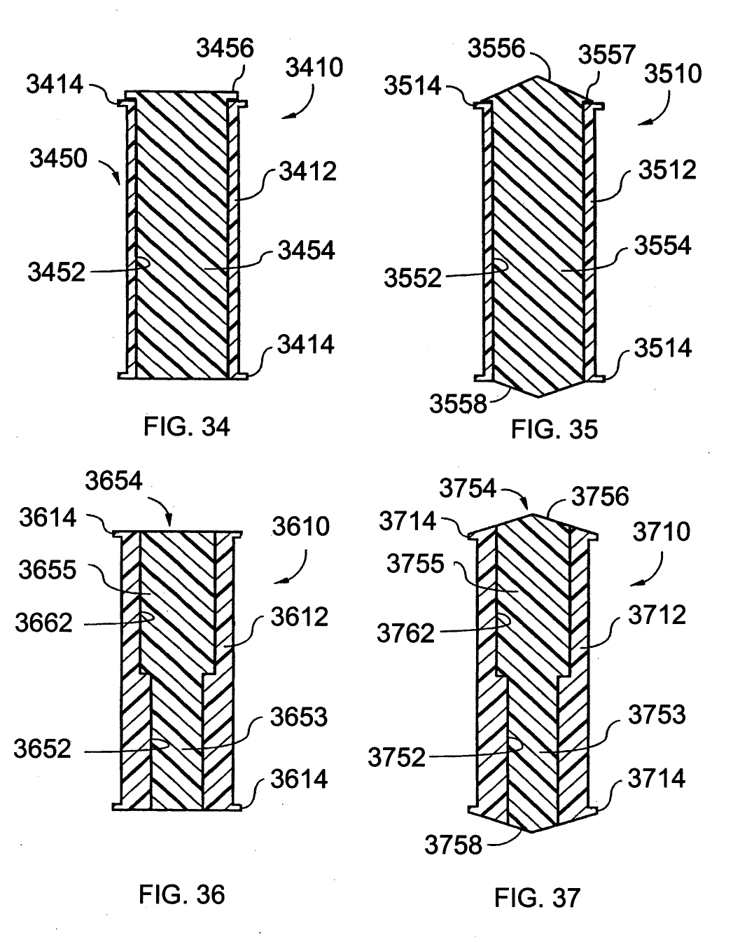

FIGS. 34-40 illustrate further two-piece float embodiments of the present disclosure wherein the sealing rings are disposed at each end of the outer sleeve and pressure relief is provided by an upwardly movable piston member.

FIG. 34 illustrates a two-piece float 3410 including a first, main body portion or sleeve 3412 having a central bore 3452 slidably receiving a second, piston-like center portion 3454. The outer body member 3412 includes a sealing ring or ridge 3414 at each end sized to engage the test tube sidewall, with an annular recess 3450 defined therebetween. The piston 3454 includes a flanged end 3456 that is greater in diameter than the central bore 3452 and less than the diameter of the sealing ridges 3414.

In operation, the piston member 3454 is fully received within the central bore 3452, with the flange 3456 abutting the upper end of the sleeve 3412. In use, the float 3410 is oriented in the tube so that the flange 3456 is located toward the top of the test tube 130, i.e., toward the stopper 140 (FIG. 1). Again, the two portions may be formed of the same material or different materials, so long as the overall specific gravity of the float 3410 is in a suitable range for buffy coat capture. In an especially preferred embodiment, the central portion 3454 is formed of a slightly higher specific gravity material than the outer portion 3412, which insures that the two portions stay together during centrifugation. Alternatively or additionally, a frictional fit is provided between the two float sections. Upon completion of centrifugation, any pressure build up in the trapped red blood cell region is alleviated by forcing the center piece 3454 upwardly.

FIG. 35 illustrates a two-piece float 3510 similar to that shown and described by way of reference to FIG. 34, but further including tapered ends for facilitating blood flow around float 3510 during centrifugation. A first, main body portion or sleeve 3512 has a central bore 3552 slidably receiving a second, piston-like center portion 3554. The outer body member 3512 includes sealing rings or ridges 3514 at opposite ends, as described above. The piston 3554 includes a tapered end 3556 including a flange 3557 sized to abut the sleeve 3512 upon insertion and restrict any further downward passage of the piston 3554. A lower end 3558 of the piston member 3554 is also tapered to facilitate flow. Centrifugal motivation and/or a frictional fit may be used to insure the two sections remain together during centrifugation.

FIG. 36 illustrates a two-piece float 3610 including a first, main body portion or sleeve 3612 having a central bore 3652 and a counterbore 3662, slidably receiving a second, piston-like center portion 3654. The outer body member 3612 includes a sealing ring or ridge 3614 as described above. The piston 3654 includes a first, smaller diameter portion sized to be received within the central bore 3652 and a second, larger diameter portion sized to be received within the counterbore 3662. The axial extent of the small diameter segment 3653 and large diameter segment 3655 may vary widely and are complimentary to that of the bore 3652 and counterbore 3662, respectively. Although the float 3610 is shown with generally flat ends, it will be recognized that the ends of the piston member 3654 and/or sleeve member 3612 may be tapered to facilitate fluid flow around the float during centrifugation.

FIG. 37 illustrates an embodiment similar to that shown in FIG. 36, having tapered ends. A two-piece float 3710 includes a first, main body portion or sleeve 3712 having a central bore 3752 and a counterbore 3762, slidably receiving a second, piston-like center portion 3754. The outer body member 3712 includes a sealing ring or ridge 3714. The piston 3754 includes a first, smaller diameter portion sized to be received within the central bore 3752 and a second, larger diameter portion sized to be received within the counterbore 3762. The tapered ends 3756 and 3758 cooperate with complimentary end ridges to form generally conical ends.

Referring to FIG. 36 and FIG. 37, during centrifugation, the float is oriented in the tube so that the counterbore and larger diameter portion are located toward the top of the test tube. As described above, the two portions may be formed of the same material or different materials and, in the preferred embodiment, the central portion (3654; 3754) is formed of a slightly higher specific gravity material than the outer sleeve (3612; 3712) insuring that the two sections stay together during centrifugation. Upon completion of centrifugation, any pressure built up in the trapped red blood cell region forces the center section (3654; 3754) upwardly.

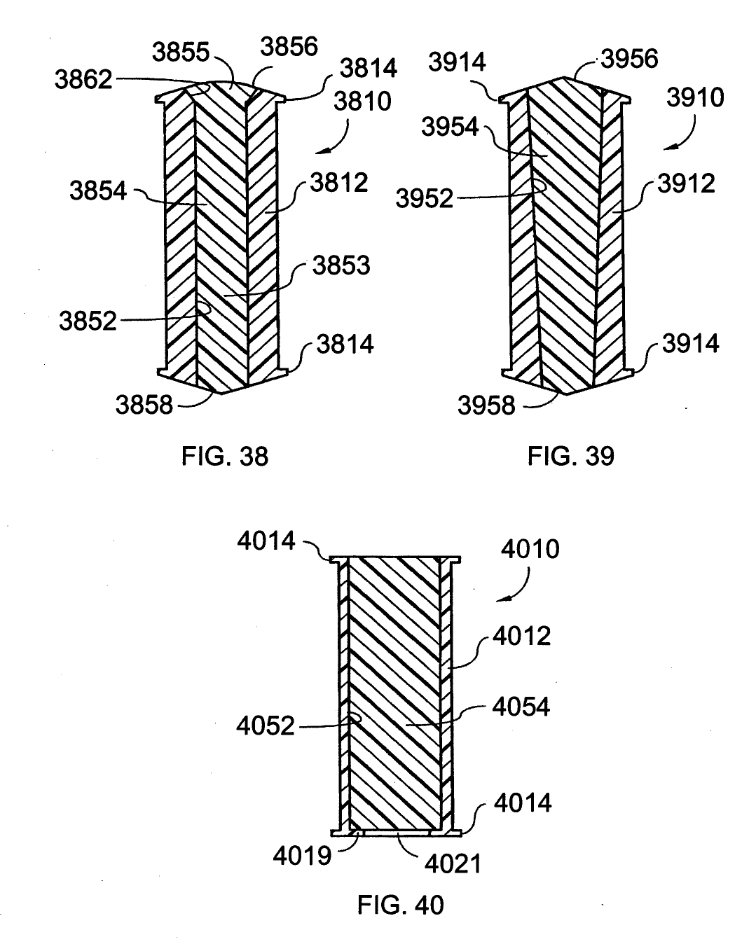

FIG. 38 illustrates yet another two-piece float embodiment 3810 including a first, main body portion or sleeve 3812 having a profiled bore comprising a central bore 3852 and an enlargement or countersink 3862 opening toward the upper end of the tube. A second, piston-like movable member 3854 includes a shaft 3853 and an enlarged head 3855, which are complimentary to and slidably received in the central bore 3852 and the countersink 3862, respectively. The outer sleeve 3812 includes sealing rings or ridges 3814 as described above. The float 3810 is shown with tapered ends 3856 and 3858, however, it will be recognized that the ends of the float 3810 may also be flat. As described above, the two sections 3812 and 3854 may be formed of the same material or different materials and, in the preferred embodiment, the movable member 3854 is formed of a slightly higher specific gravity material than the outer sleeve 3812, insuring that the two sections stay together during centrifugation.

FIG. 39 illustrates a further two-piece separator float embodiment 3910 including a first, main body portion or sleeve 3912 having a tapered internal passage 3952 which widens toward the upper end 3956 of the float. A central, movable member 3954 complimentary to the bore 3952 is slidably received therein. The outer sleeve 3912 includes sealing rings or ridges 3914. The separator float ends 3956 and 3958 are illustrated as tapered, although flat ends are also contemplated. The two sections 3912 and 3954 may be formed of the same material or different materials, again, with the movable member 3954 preferably formed of a slightly higher specific gravity material to keep the float sections together during centrifugation.

FIG. 40 illustrates a further two-piece separator float embodiment 4010 including a first, main body portion or sleeve 4012 having an central passage or bore 4052 which terminates in an annular seat 4019 formed at a lower end of the float 4010 and defining an opening 4021 into the bore 4052. A piston-like movable member 4054 is slidably received within the bore 4052, abutting the annular seat 4019. The outer sleeve 4012 includes sealing rings or ridges 4014. The separator float 4010 is depicted with flat ends, although tapered ends are also contemplated. Optionally, the movable member 4054 may contain a narrow diameter portion (not shown) on the lower end thereof sized to be received in the aperture 4021, e.g., to provide a flush and/or tapered surface to facilitate flow therepast during centrifugation. The two sections 4012 and 4054 may be formed of the same material or different materials; preferably, the movable member 4054 is formed of a slightly higher specific gravity material to keep the float sections together during centrifugation.

Each of the float embodiments of FIGS. 30-40, which have been illustrated with end sealing rings and without additional tube supporting members for ease of demonstration, may be further modified by the further incorporation of any of the tube support features as shown in the earlier figures, such as annular bands, segmented bands, helical bands, axial splines, rounded protrusions, spikes, facets, and combinations thereof. Likewise, the separator float embodiments are depicted herein having either flat or the preferred conical ends; however, many other geometrical shapes providing a curved, sloping, and/or tapered surface to facilitate density-motivated cell and float movement during centrifugation are contemplated. Exemplary modified end shapes include, for example, frustoconical, convex or dome-shaped, and other tapered shapes.

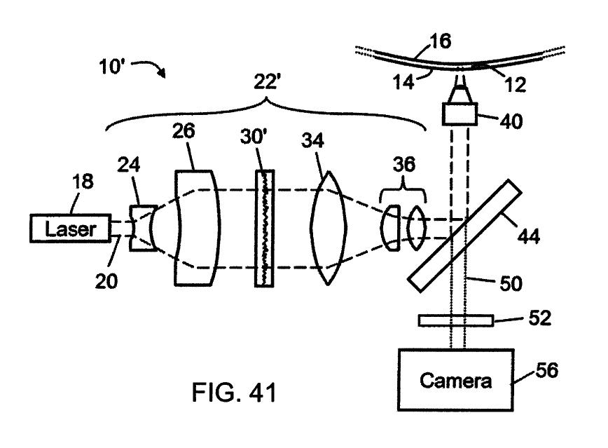

Referring back to FIG. 2, some variations that lead to other suitable microscope systems are described in FIGS. 41-43. FIG. 41 shows a microscope system 10′ that is similar to the microscope system 10 of FIG. 2, except that the optical train 22′ differs in that the stationary beam homogenizer 30 of FIG. 2 is replaced by a stationary diffuser 30′. The diffuser 30′ may, for example, be a holographic diffuser available, for example, from Physical Optics Corporation (Torrance, Calif.). Such holographic diffusers employ a hologram providing randomizing non-periodic optical structures that diffuse the light to impart improved spatial uniformity. However, the diffusion of the light also imparts some concomitant beam divergence. Typically, stronger diffusion of the light tend to impart more spatial uniformity, but also tends to produce greater beam divergence. Holographic diffusers are suitably classified according to the full-width-at-half-maximum (FWHM) of the divergence angle, with larger divergence angles typically providing more diffusion and greater light uniformity, but also leading to increased light loss in the microscope system 10′ due to increased beam divergence.

In some embodiments of the microscope system 10′, the diffuser 30′ is a low-angle diffuser having a FWHM less than or about 10°. Lower angle diffusers are generally preferred to provide less divergence and hence better illumination throughput efficiency; however, if the divergence FWHM is too low, the diffuser will not provide enough light diffusion to impart adequate beam uniformity. Low diffusion reduces the ability of the diffuser 30′ to homogenize the Gaussian distribution, and also reduces the ability of the diffuser 30′ to remove speckle.

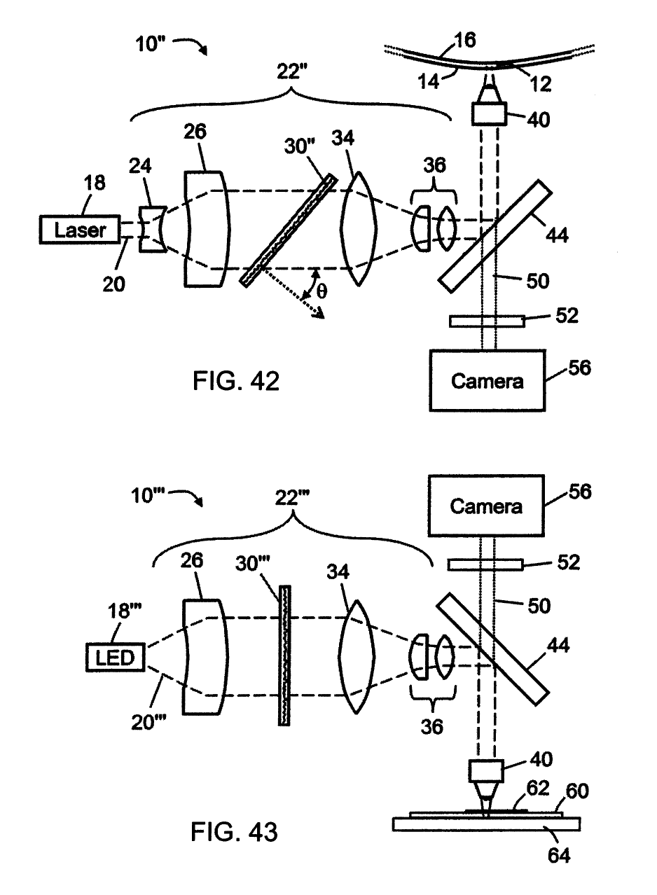

With reference to FIG. 42, another embodiment microscope system 10″ is similar to the microscope system 10′, and includes an optical train 22″ that employs a diffuser 30″ similar to the diffuser 30′ of the microscope system 10′. However, the diffuser 30″ is tilted at an angle θ respective to the optical path of the optical train 22″ so as to substantially reduce a speckle pattern of the source light 20. Without being limited to any particular theory of operation, it is believed that the tilting shifts the speckle pattern to higher spatial frequencies, in effect making the speckle size smaller. The speckle size is spatially shifted by the tilting such that the frequency-shifted speckle is substantially smaller than an imaging pixel size.

In some embodiments, a tilt angle θ of at least about 30° respective to the optical path of the optical train 22″ is employed, which has been found to substantially reduce speckle for diffusers 30″ having a FWHM as low as about 5°. On the other hand, tilt angles θ of greater than about 45° have been found to reduce illumination throughput efficiency due to increased scattering, even for a low-angle diffuser having a FWHM of 5°.

In FIG. 43, a microscope system 10′″ includes a light emitting diode (LED) 18′″ as the light source, rather than the laser 18 used in the previous microscope systems 10, 10′, 10″. Because the LED 18′″ outputs diverging source light 20′″ rather than a collimated laser beam, an optical train 22′″ is modified in that the beam-expanding concave lens 24 is suitably omitted, as shown in FIG. 43. Alternatively, a lens can be included in the position of the lens 24, but selected to provide a suitable divergence angle adjustment for collimation by the collimating lens 26. The optical train 10′″ employs a diffuser 30′″ similar to the diffusers 30′, 30″. The LED 18′ outputs incoherent light, and so speckle is generally not present. However, the output of the LED 18′″ typically does have a non-Gaussian distribution, for example a Lambertian distribution. In view of these characteristics of the source light 20″, the diffuser 30′″ is not tilted, and in some cases the diffuser 30″ can have a smaller divergence angle FWHM than the untilted diffuser 30′ used to impart spatial uniformity to the laser beam source light 20 in the microscope system 10′ of FIG. 41.

The microscope system 10′ of FIG. 43 further differs from the microscope systems 10, 10′, 10″ in that the microscope system 10′″ images a sample disposed on a planar slide 60, which is optionally covered by an optional cover glass 62. The slide 60 is disposed on an x-y planar translation stage 64 to enable scanning across the sample. It will be appreciated that the LED 18″ and optical train 22′″ are also suitable for imaging the buffy coat sample disposed in the annular gap 12 between the light-transmissive test tube wall 14 and float wall 16 shown in FIG. 41 and FIG. 42. Conversely, it will be appreciated that the laser 18 and optical train 22, 22′, 22″ are also suitable for imaging the planar sample on the slide 60 shown in FIG. 43.

The optical trains 22, 22′, 22″, 22′″ have components which are stationary in the sense that the components are not rotated, relatively oscillated, or otherwise relatively moved. It is, however, contemplated to move the optical train and the objective 40 as a whole, and/or to include beam-steering elements, or so forth, to enable relative scanning of the field of view respective to the sample.

Suitable microscope systems for imaging an annular sample contained in or supported by a test tube have been described in FIG. 2 and FIGS. 41-43. The annular gap 12 typically has a thickness that is substantially larger than a depth of view of the microscope objective 40. The test tube wall 12 and float wall 16 are typically not uniform across the entire surface of the test tube or float. While the microscope objective 40 typically has an adjustable depth of focus (adjusted by moving internal optical components and/or by moving the objective 40 toward or away from the test tube wall 12), the range of adjustment is limited. Accordingly, the test tube should be held such that the surface proximate to the objective 40 is at a well-defined distance away from the objective 40 as the test tube is rotated and as the objective 40, or the test tube, is translated along a tube axis.

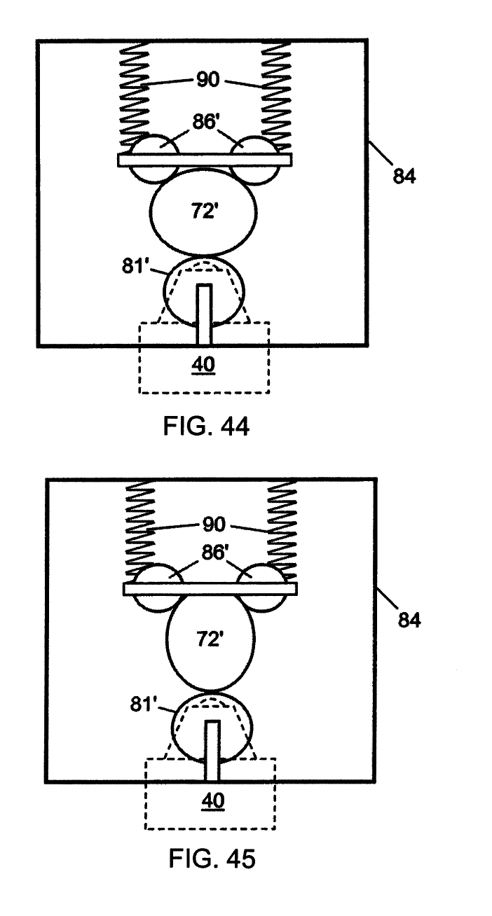

A variation on the test tube holder of FIGS. 3-6 is shown in FIG. 44 and FIG. 45. A modified test tube 72′ having an elliptical cross section is more precisely aligned by employing a set of three bearings per supported float ridge, in which the three bearings include only one alignment bearing 81′ and two or more biasing bearings 86′. The alignment bearing 81′ is at the same radial position as the objective 40 (shown in phantom in FIG. 44 and FIG. 45). As the elliptical test tube 12′ rotates, the imaged side that is biased against the alignment bearings 81′ remains precisely aligned with the radially coincident objective 40 whether the imaged side correspond to the short axis of the elliptical test tube 72′ (FIG. 44), or whether the imaged side correspond to the long axis of the elliptical test tube 72′ (FIG. 45).

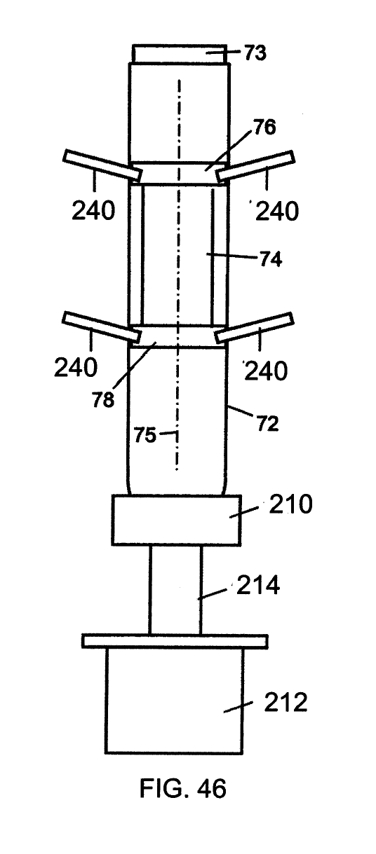

With reference to FIG. 46, in another variation, bearings 240 are tilted respective to the tube axis 75 of the test tube 72 to impart force components parallel with the tube axis 75 to push the test tube 72 into the rotational coupling 210. In this arrangement, the spring loaded cap 216 is optionally omitted, because the tilting of the bearings 240 opposes translational slippage of the test tube 72 during rotation.

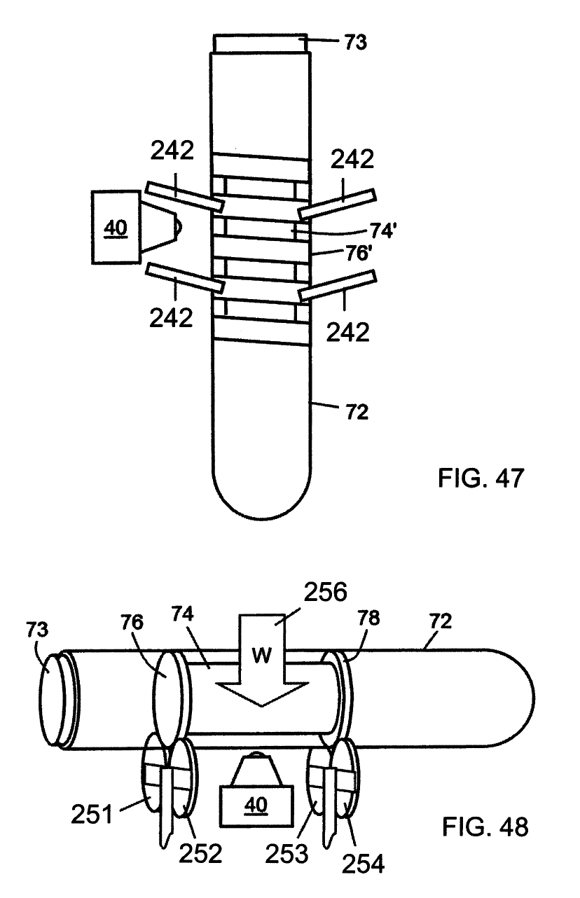

With reference to FIG. 47, in another variation, a modified float 74′ includes spiral ridges 76′, and tilted bearings 242 are spaced along the tube axis 75 in accordance with the spiral pitch to track the spiraling sealing ridges 76′ responsive to rotation of the test tube 72. In this approach, the tilted bearings 242 impart a force that causes the test tube 72 to translate along the tube axis 75, so that the objective 40 can be maintained at a fixed position without translating while scanning annular gap 12′. In this approach, the roller bearings 242 are suitably motorized to generate rotation of the test tube 72. That is, the roller bearings 242 also serve as the rotational coupling.

With reference to FIG. 48, in another variation, the mechanical bias can be provided by a mechanism other than biasing bearings. Here, the test tube 72 is arranged horizontally resting on alignment bearings 251, 252, 253, 254 with the objective 40 mounted beneath the test tube 72. A weight of the test tube 72 including the float 74 (said weight diagrammatically indicated by a downward arrow 256) provides as the mechanical bias pressing the test tube 72 against the alignment bearings 251, 252, 253, 254. In other contemplated embodiments, a vacuum chuck, positive air pressure, magnetic attraction, or other mechanical bias is employed to press the test tube against the alignment bearings. The alignment bearings 251, 252, 253, 254 can be rotated mechanically so that the alignment bearings 251, 252, 253, 254 serve as the rotational coupling, or a separate rotational coupling can be provided.

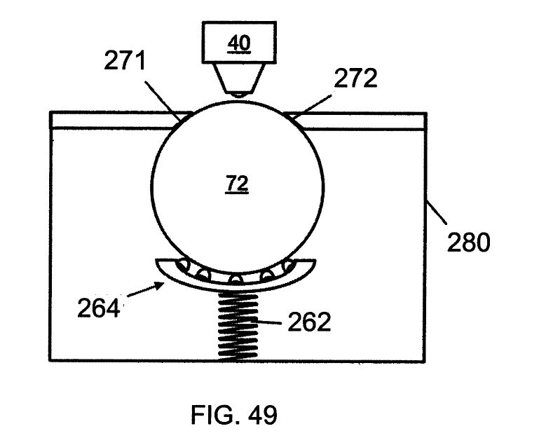

With reference to FIG. 49, the bearings can be other than roller bearings. For example, the bearings can be rollers, ball bearings, or bushing surfaces. In the variant test tube holder shown here, a housing 280 provides an anchor for a spring 262 that presses a set of biasing ball bearings 264 against the test tube 72 to press the test tube 72 against alignment bearings 271, 272 defined by bushing surfaces of the housing 280. Other types of bearings can be used for the biasing and/or alignment bearings that support the test tube as it rotates.

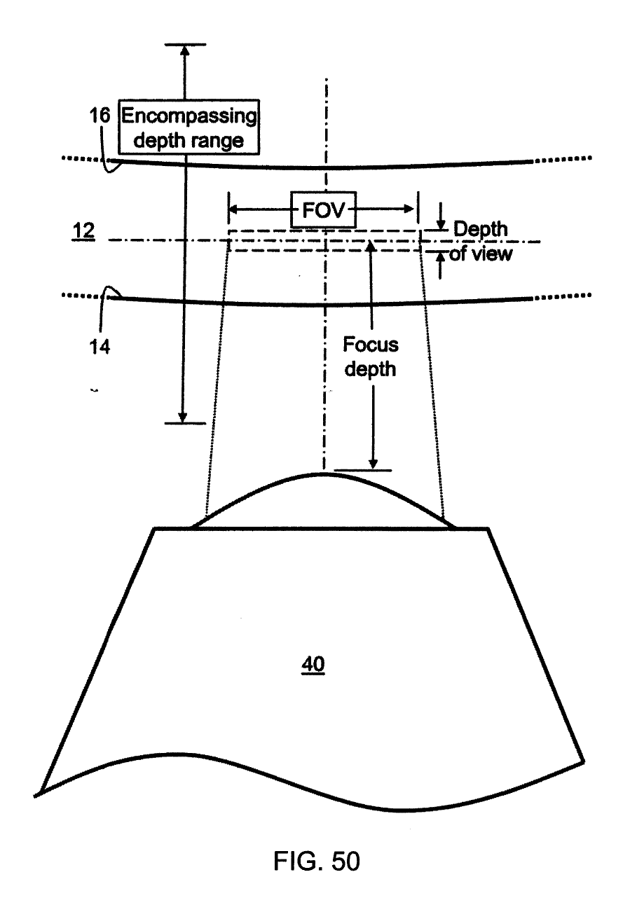

Suitable processing approaches for identifying or quantifying fluorescent dye tagged cells in an annular biological fluid layer are now described in FIGS. 50-56.

With reference to FIG. 50, certain measurement parameters are diagrammatically illustrated. The objective 40 images over a field of view (FOV) and over a depth of view located at a focus depth. Here, the focus depth is indicated respective to the objective 40; however, the focus depth can be denoted respective to another reference. In some embodiments, the depth of view of the objective 40 is about 20 microns, while the annular gap 12 between the test tube wall 14 and the float wall 16 is about 50 microns. However, the depth of focus corresponding to the annular gap 12 can vary substantially due to non-uniformities in the test tube and/or the float or other factors. It is expected that the annular gap 12 is located somewhere within an encompassing depth range. In some embodiments, an encompassing depth range of 300 microns has been found to be suitable. These dimensions are examples, and may be substantially different for specific embodiments depending upon the specific objective 40, light-transmissive test tube, float, the type of centrifuging or other sample processing applied, and so forth.

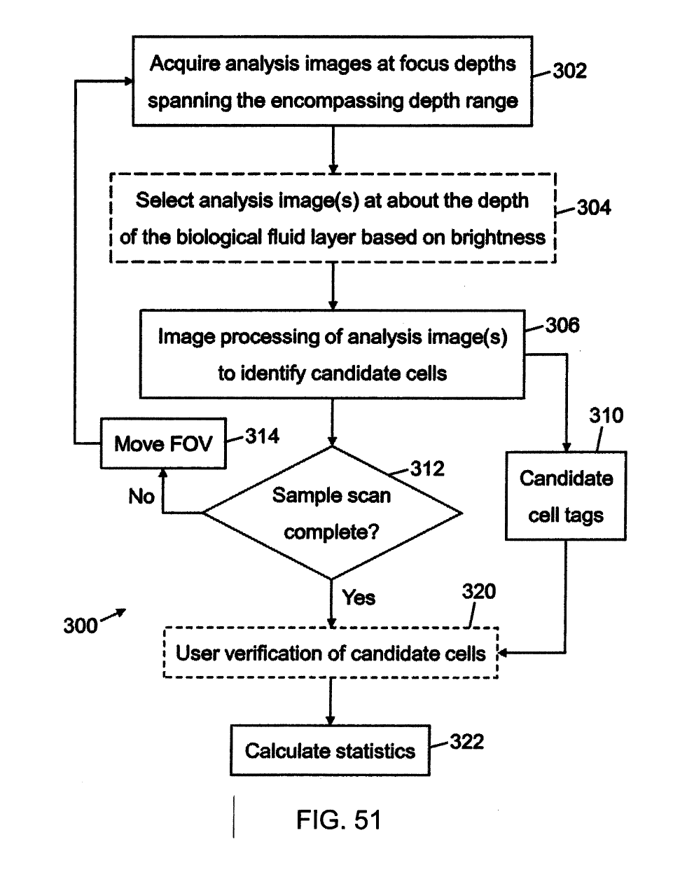

With reference to FIG. 51, one suitable data acquisition approach 300 is diagrammatically shown. In process operation 302, analysis images are acquired at a plurality of focus depths spanning the encompassing depth range. To avoid gaps in the depth direction, the number of analysis images acquired in the operation 302 should correspond to at least the encompassing depth range divided by the depth of view of the objective 40.

In some embodiments, the analysis images are processed in optional operation 304 to identify one or more analysis images at about the depth of the biological fluid layer (such as the buffy layer) based on image brightness. This optional selection takes advantage of the observation that typically the fluorescent dye produces a background fluorescence that is detected in the acquired analysis images as an increased overall image brightness. Image brightness can be estimated in various ways, such as an average pixel intensity, a root-mean-square pixel intensity, or so forth.

In an image processing operation 306, the analysis images, or those one or more analysis images selected in the optional selection operation 304, are processed using suitable techniques such as filtering, thresholding, or so forth, to identify observed features as candidate cells. The density of dye-tagged cells in the biological fluid layer is typically less than about one dye-tagged cell per field of view. Accordingly, the rate of identified candidate cells is typically low. When a candidate cell is identified by the image processing 306, a suitable candidate cell tag is added to a set of candidate cell tags 310. For example, a candidate cell tag may identify the image based on a suitable indexing system and x- and y-coordinates of the candidate cell feature. Although the density of rare cells is typically low, it is contemplated that the image processing 306 may nonetheless on occasion identify two or more candidate cells in a single analysis image. On the other hand, in some analysis images, no candidate cells may be identified.

At a decision point 312, it is determined whether the sample scan is complete. If not, then the field of view is moved in operation 314. For example, the field of view can be relatively scanned across the biological fluid sample in the annular gap 12 by a combination of rotation of the test tube 72 and translation of the objective 40 along the test tube axis 75. Alternatively, using the tube holder of FIG. 47, scanning is performed by moving the test tube 72 spirally. For each new field of view, the process operations 302, 304, 306 are repeated.

Once the decision point 312 indicates that the sample scan is complete, a user verification process 320 is optionally employed to enable a human analyst to confirm or reject each cell candidacy. If the image processing 306 is sufficiently accurate, the user verification process 320 is optionally omitted.

A statistical analysis 322 is performed to calculate suitable statistics of the cells confirmed by the human analyst. For example, if the volume or mass of the biological fluid sample is known, then a density of rare cells per unit volume or per unit weight (e.g., cells/milliliter or cells/gram) can be computed. In another statistical analysis approach, the number of confirmed cells is totaled. This is a suitable metric when a standard buffy sample configuration is employed, such as a standard test tube, standard float, standard whole blood sample quantity, and standardized centrifuging processing. The statistical analysis 322 may also include threshold alarming. For example, if the cell number or density metric is greater than a first threshold, this may indicate a heightened possibility of cancer calling for further clinical investigation, while if the cell number or density exceeds a second, higher threshold this may indicate a high probability of the cancer calling for immediate remedial medical attention.

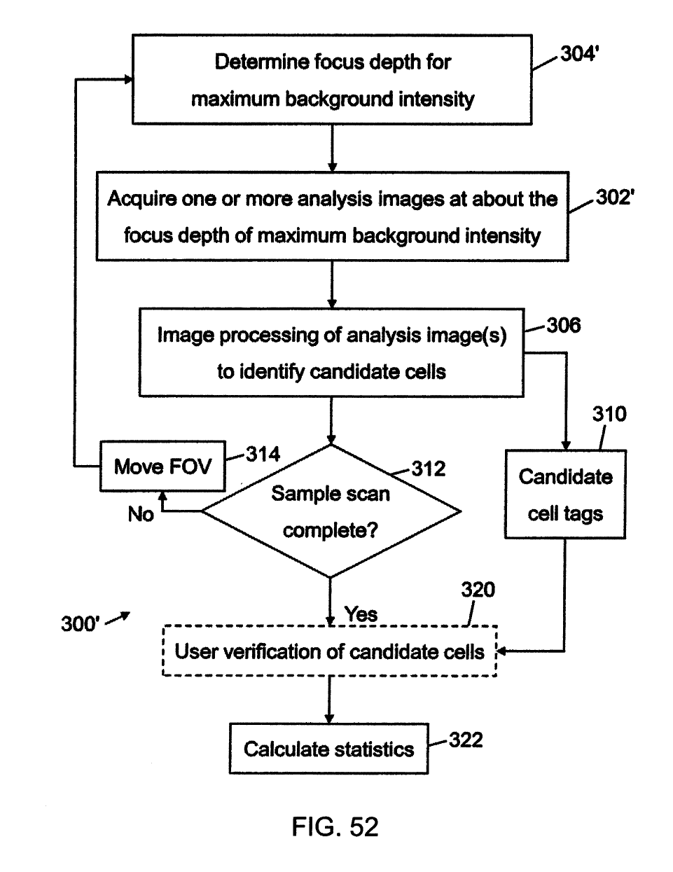

With reference to FIG. 52, a modified acquisition approach 300′ is diagrammatically shown. In modified process operation 304′, the focus depth for maximum background fluorescence intensity is first determined using input other than analysis images, followed by acquisition 302′ of one or a few analysis images at about the focus depth for maximum background fluorescence. For example, the search process 304′ can be performed by acquiring low resolution images at various depths. To avoid gaps in the depth direction, the number of low resolution images acquired in the operation 304′ should correspond to at least the encompassing depth range divided by the depth of view of the objective 40. In another approach, a large-area brightness sensor (not shown) may be coupled to the captured fluorescence 50 (for example, using a partial mirror in the camera 56, or using an intensity meter built into the camera 56) and the focus of the objective 40 swept across the encompassing depth range. The peak signal of the sensor or meter during the sweep indicates the focus providing highest brightness.

With the depth of the biological fluid sample determined by the process operation 304′, the acquisition process 302′ acquires only one or a few analysis images at about the identified focus depth of highest brightness. To ensure full coverage of the biological fluid layer, the number of acquired analysis images should be at least the thickness of the annular gap 12 divided by the depth of view of the objective 40. For example, if the annular gap 12 has a thickness of about 50 microns and the depth of view is about 20 microns, then three analysis images are suitably acquired—one at the focus depth of highest brightness, one at a focus depth that is larger by about 15-25 microns, and one at a focus depth that is smaller by about 15-25 microns.

An advantage of the modified acquisition approach 300′ is that the number of acquired high resolution analysis images is reduced, since the focus depth is determined prior to acquiring the analysis images. It is advantageous to bracket the determined focus depth by acquiring analysis images at the determined focus depth and at slightly larger and slightly smaller focus depths. This approach accounts for the possibility that the rare cell may be best imaged at a depth that deviates from the depth at which the luminescence background is largest.

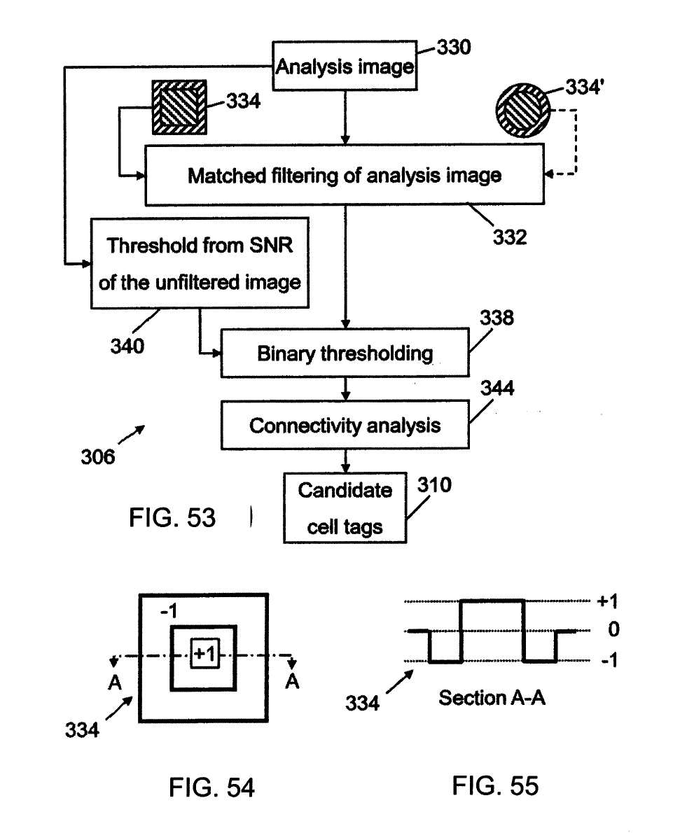

With reference to FIG. 53, a suitable embodiment of the image processing 306 is described, which takes advantage of a priori knowledge of the expected rare cell size to identify any cell candidates in an analysis image 330. In a matched filtering process 332, a suitable filter kernel is convolved with the image. The matched filtering 332 employs a filter kernel having a size comparable with the expected size of an image of a rare cell in the analysis image 330.

With continuing reference to FIG. 53 and with brief further reference to FIG. 54 and FIG. 55, in some embodiments a square filter kernel 334 is employed. The kernel 334 includes a central positive region of pixels each having a value of +1, and an outer negative region of pixels each having a value of −1. The area of the positive region should be about the same size as the area of the negative region. Points outside of either the inner or outer region have pixel values of zero. Optionally, other pixel values besides +1 and −1 can be used for the inner and outer regions, respectively, so as to give the filter a slightly positive or slightly negative response.

With continuing reference to FIG. 53, the matched filtering removes or reduces offsets caused by background illumination, and also improves the signal-to-noise ratio (SNR) for rare cells. The signal is increased by the number of points in the positive match area, while the noise is increased by the number of points in both the positive and negative match areas. The gain in SNR comes from the fact that the signal directly adds, while the noise adds as the root-mean-square (RMS) value or as the square root of the number of samples combined. For a filter with N positive points and N negative points, a gain of N/√(2N) or √(N/2) is obtained.

The square filter kernel 334 is computationally advantageous since its edges align with the x- and y-coordinate directions of the analysis image 330. A round filter kernel 334′ or otherwise-shaped kernel is optionally used in place of the square filter kernel 334. However, the round filter kernel 334′ is more computationally expensive than the square filter kernel 334. Another advantage of the square filter kernel 334 compared with the round filter kernel 334′ is that the total filter edge length of the square filter 334 is reduced from twice the detection size to 1.414 times the detection size. This reduces edge effects, allowing use of data that is closer to the edge of the analysis image 330.

The size of the filter kernel should be selected to substantially match the expected image size of a dye-tagged cell in the analysis image 330 to provide the best SNR improvement. For example, the square filter kernel 334 with a positive (+1) region that is ten pixels across is expected to provide the best SNR improvement for a cell image also having a diameter of about ten pixels. For that matched case, the signal is expected to increase by about a factor of 78 while the noise is expected to increase by about a factor of 14, providing a SNR improvement of about 5.57:1. On the other hand, the SNR improvement for a smaller eight pixel diameter cell using the same square filter is expected to be about 3.59:1. The SNR improvement for a larger fourteen pixel diameter cell using the same square filter is expected to be about 3.29:1.

The matched filter processing 332 can be implemented in various ways. In one approach, each point in the input image is summed into all points in the output image that are in the positive inner region. Then all the points in the output image that are in the outer negative region but not in the inner positive region are subtracted off. Each point in the input image is touched once, while each point in the output image is touched the outer-box pixel area count number of times.

In another suitable approach, for each point in the output image, all points from the input image that are within the positive inner box are read and summed. All points outside the positive inner box but within the negative outer box are then subtracted. While each output image pixel is touched only once, each input image pixel is touched by the outer-box pixel count.

In another suitable approach, two internal values are developed for the current row of the input image: a sum of all points in the row in the negative outer box distance, and a sum of all points in the row in the inner positive box distance. All output image column points at the current row have the input image sum of all points in the outer-box subtracted from them. All the output image column points within the inner positive box get the sum of the input image row points in the inner positive box distance added in twice. The row sums can be updated for the next point in the row by one add and one subtract. This reduces the execution cost to be on the order of the height of the filter box.

In the matched filter processing 332, various edge conditions can be employed. For example, in one approach, no output is produced for any point whose filter overlaps an edge of the analysis image 330. This approach avoids edge artifacts, but produces an output image of reduced usable area. In another suitable example edge condition, a default value (such as zero, or a computed mean level) is used for all points off the edge.

With continuing reference to FIG. 53, binary thresholding processing 338 is applied after the matched filtering 332. A difficulty in performing the thresholding 338 is selection of a suitable threshold value. Threshold selection is complicated by a likelihood that some analysis images will contain no cells, or only a single cell, or only a couple or few cells. In one approach, a the threshold is selected as a value that is a selected percentage below the peak pixel intensity seen in the filtered data. However, this threshold will cause noise to be detected when no cells are present, since in that case the peak pixel value will be in the noise. Another approach is to use a fixed threshold. However, a fixed threshold may be far from optimal if the background intensity varies substantially between analysis images, or if the matched filtering substantially changes the dynamic range of the pixel intensities.

In the illustrated approach, the threshold is determined by processing 340 based on the SNR of the unfiltered analysis image 330. By first determining the standard deviation of the input image, the expected noise at the filter output can be computed. The noise typically rises by the square root of the number of pixels summed, which is the outer-box area in pixel counts. In some embodiments, the threshold is set at approximately 7-sigma of this noise level. As this filter does not have an exact zero DC response, an appropriate mean level is also suitably summed to the threshold.

The thresholding 338 produces a binary image in which pixels that are part of a cell image generally have a first binary value (e.g., “1”) while pixels that are not part of a cell image generally have second binary value (e.g., “0”). Accordingly, connectivity processing 344 is performed to identify a connected group of pixels of the first binary value corresponding to a cell. The connectivity analysis 344 aggregates or associates all first binary value pixels of a connected group as a cell candidate to be examined as a unit. The center of this connected group or unit can be determined and used as the cell location coordinates in the candidate cell tag.

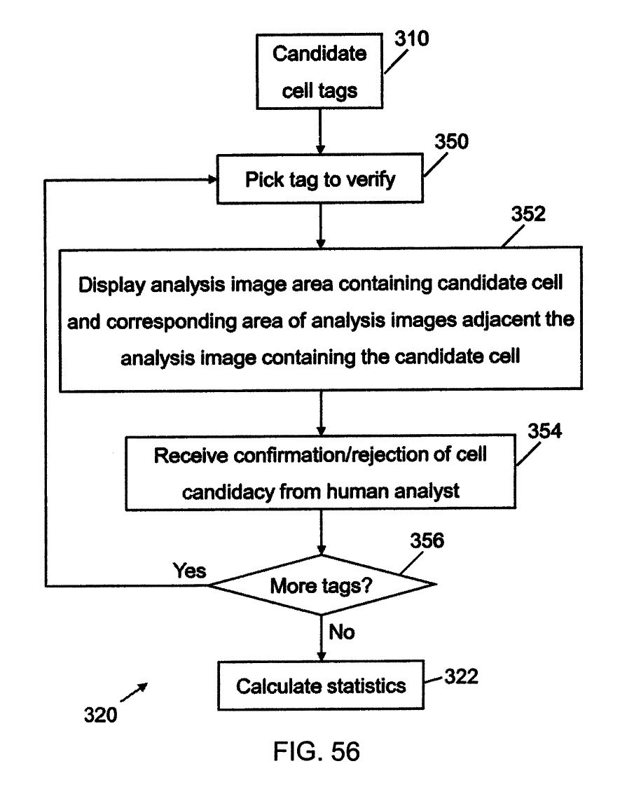

With reference to FIG. 56, a suitable embodiment of the optional user verification processing 320 is described. A tag is selected for verification in a selection operation 350. In a display operation 352, the area of the analysis image containing the candidate cell tag is displayed, optionally along with the corresponding area of analysis images adjacent in depth to the analysis image containing the candidate cell. Displaying the analysis images that are adjacent in depth provides the reviewing human analyst with additional views which may fortuitously include a more recognizable cell image than the analysis image in which the automated processing 306 detected the cell candidate. The human analyst either confirms or rejects the candidacy in operation 354. A loop operation 356 works though all the candidate cell tags to provide review by the human analyst of each candidate cell. The statistical analysis 322 operates on those cell candidate tags that were confirmed by the human analyst.

Several additional different test tubes and separator floats are contemplated for use with the methods of the present disclosure. These floats and test tubes can be used as both the assay test tube or the control test tube.

Referring now to FIG. 1, several different means are possible to remove the buffy coat layers/constituents from the sample tube. In some embodiments, the blood sample and float are introduced into the sample tube, the tube is centrifuged, and the rotational speed is then reduced to trap the buffy coat constituents in the annular volume. Next, at least one support member 114 is welded to the sidewall 136 of the sample tube 130. This weld traps the buffy coat layers within the annular volume 150, which can now be considered an enclosed toroid and in which the buffy coat layers are separated from the plasma and/or red blood cells.

The weld may be continuous about a circumference of the welded member, i.e. the perimeter where the support member contacts the sidewall. The weld may also be discontinuous, i.e. there are gaps in the weld. In particular embodiments, the welding is performed ultrasonically. Ultrasonic welding is an industrial technique commonly used for plastics, whereby high-frequency ultrasonic acoustic vibrations are locally applied to two items being held together to create a solid-state weld between the two items. The term “welding” is used here to indicate the action of joining the float with the sample tube in a specific location, and is synonymous with melting.

In some embodiments, a flexible sleeve is placed in the sample tube, and the float and blood sample are then placed into the flexible sleeve. In these embodiments, the at least one support member 114 may be welded to the sleeve.

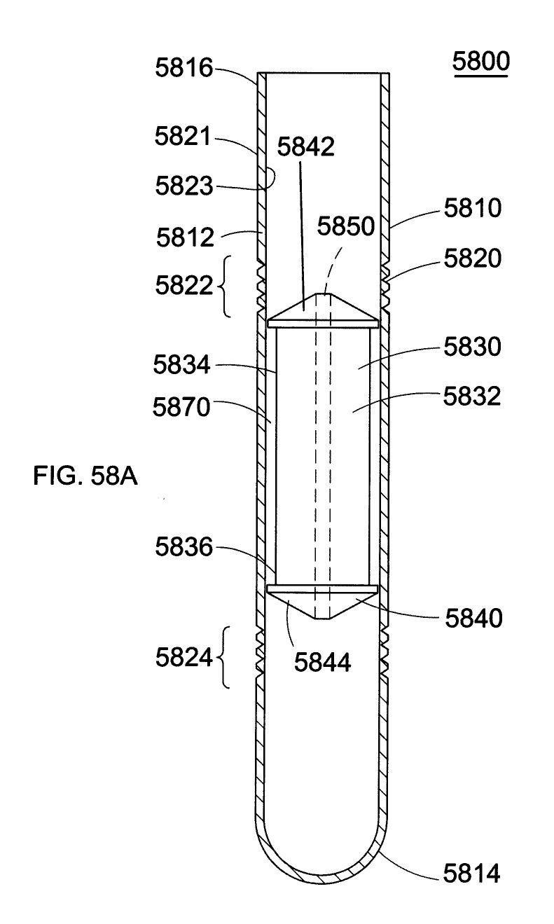

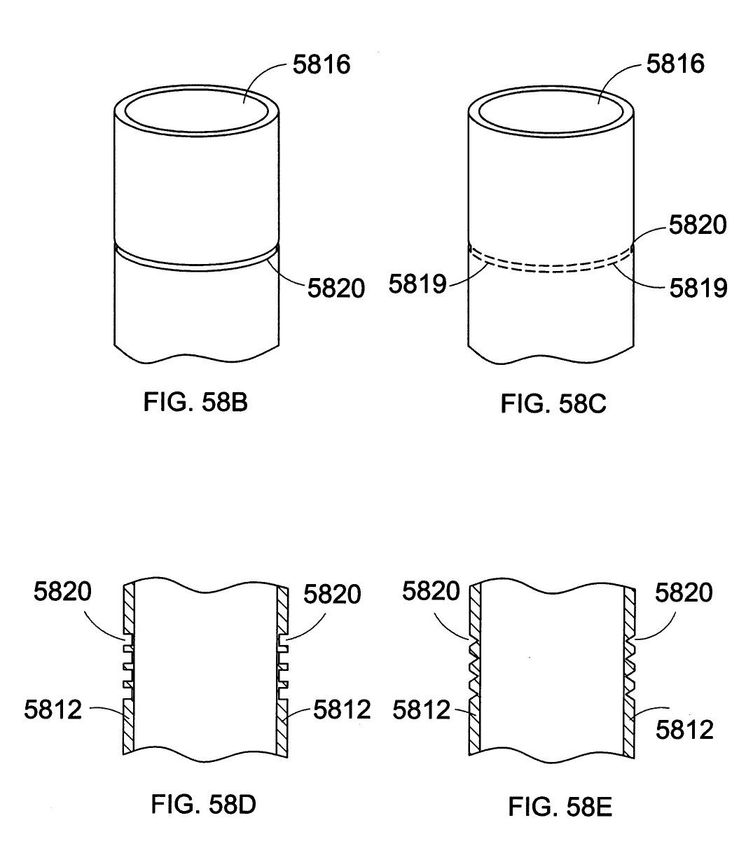

Referring to FIG. 58A, in particular embodiments, the separator float 5830 includes a main body portion 5832 having a top end 5834 and a bottom end 5836. A top support member 5842 extends radially from the top end 5834, and a bottom support member 5844 extends radially from the bottom end 5836. The sidewall 5812, main body portion 5832, top support member 5842, and bottom support member 5844 together define an annular volume 5870. In preferred embodiments, both the top and bottom support members are welded to the sample tube. An axial bore 5850 is present for pressure relief of the red blood cell portion below the buffy coat layers.

The blood separation apparatus 5800 shown in FIG. 58A also shows another exemplary embodiment of a sample tube 5810. The sample tube 5810 includes a sidewall 5812, a first, closed end 5814, a second, open end 5816, and circumferential notches 5820. A circumferential notch is formed by one or more grooves that lie substantially within the same plane, that plane being perpendicular to the sidewall of the tube. A first set 5822 of notches is located above the float 5830 and a second set 5824 of notches is located below the float. Each set is shown here in FIG. 58A with three notches, but this number can vary and is generally between one and four notches in each set. The sample tube is broken along one or more notches to get access to the float and the buffy coat layers trapped in the annular volume 5870.

FIGS. 58B-58E illustrate different variations of the notches. In FIG. 58B, the depicted set has one notch which is formed by one continuous groove 5819, i.e. the notch is continuous around the circumference. In FIG. 58C, the depicted notch is formed by a set of short grooves 5819, i.e. the notch is discontinuous around the circumference. In FIG. 58D, the set has two notches, each of which is rectangularly shaped, while in FIG. 58E, the notch is triangularly shaped. In other words, the notch may have a triangular or rectangular axial cross-section. Other notch shapes, such as U-shaped, are also contemplated. These forms may be useful in directing how the tube breaks. Although the notches 5820 in FIG. 58A are on the exterior surface 5821 of the sample tube 5810, the notches could be located on the interior surface 5823 of the sample tube 5810 and should not interfere with axial movement of the float. The sample tube may only have a single notch in some embodiments. However, in desirable embodiments, the sample tube 5810 comprises a first set 5822 of notches and a second set 5824 of notches, which divide the tube into three volumes (upper, central, lower).

Again, the blood sample and float are introduced into the sample tube 5810, the tube is centrifuged, and the rotational speed is then reduced to trap the buffy coat constituents in the annular volume. Methods using the sample tube 5810 further include breaking the sample tube 5810 at at least one of the one or more notches 5820 to obtain a section of the tube 5810 containing the float 5830 and annular volume 5870 which contains the buffy coat constituents. In certain preferred embodiments, at least one notch in the first set 5822 of notches above the float 5830 and at least one notch in the second set 5824 of notches below the float 5830 are broken. The tube may be broken, for example, by simple twisting or snapping. The annular volume 5870 can be examined to identify target cells either before or after breaking the tube, as desired.

Desirably, the amount of blood introduced into the sample tube is controlled so that after centrifugation, the float 5830 is located in the middle volume 5825 of the tube 5810. As seen in FIG. 58A, no notches are present along the axial length 5831 of the float. This result aids in ensuring that breakage and consequent loss of the buffy coat layers does not occur.

Sealing glass ampules are known that allow the lower bulb, containing a sample, to be sealed off. Typically, such ampules have a constriction to which heat is applied to soften the glass. The glass collapses, forming the seal, and the lower bulb is gently pulled away from the remainder of the tube. Such sealed ampules differ from the sample tube of FIG. 58A in that the glass material of the tube completely surrounds the sample, whereas here the separator float itself provides one or two surfaces that surround the buffy coat sample. In addition, such sealed ampules typically seal their sample in the lower bulb, i.e. the ampule is divided into two volumes. In contrast, the sample tube of FIG. 58A can be divided into three volumes. Finally, the breaking of the sample tube is easier and less time-consuming than heating and sealing the ampule.

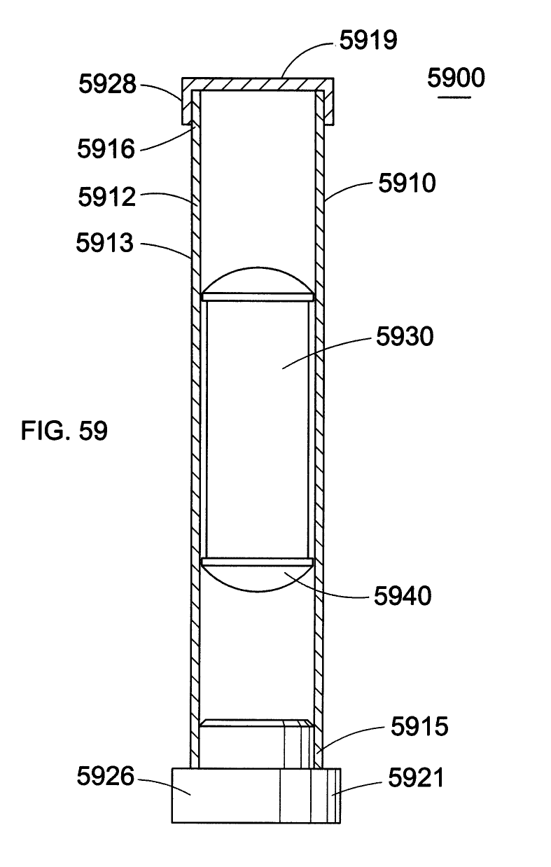

FIG. 59 shows another concept of a blood separation apparatus 5900 including a sample tube 5910 and a separator float 5930. The sample tube 5910 is formed from a sidewall 5912, shown here as a cylinder 5913, though the tube may generally have any lateral cross-sectional shape. The sidewall defines a first open end 5915 and a second open end 5916, which are opposite each other. A first closure device 5926 closes the first end 5915 and a second closure device 5928 closes the second end 5916. The closure device can be an exterior cap, such as cap 5919, that does not penetrate into the cylinder, or an interior cap such as a stopper 5921, that does penetrate into the cylinder, or any combination thereof.

When the apparatus of FIG. 59 is used, the first closure device 5926 seals the first end 5915 during centrifugation. The second end 5916 may be sealed with the second closure device 5928 or left open. At the end of centrifugation, the first closure device 5926 and/or the second closure device 5928 are then removed to access the float and the expanded buffy coat layer. In particular, the two-cap design advantageously allows the plasma and the red blood cells to be drained from the sample tube 5910, leaving the expanded buffy coat layer in the cylinder 5913 to be analyzed. If desired, this concept can also be combined with the notches 5820 described above, so that a sample tube has circumferential notches and two open ends, the tube being broken at the notches after draining the plasma and the red blood cells.

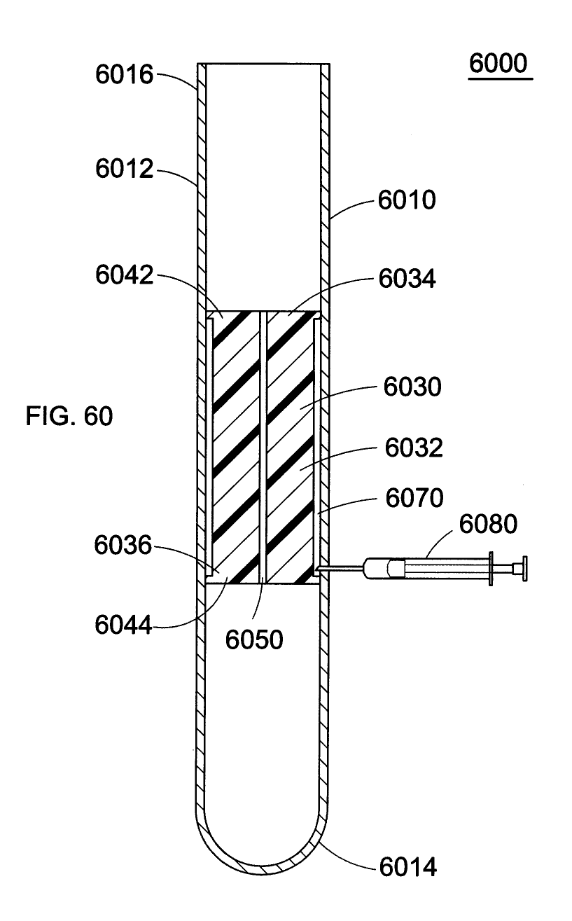

The buffy coat constituents can also be withdrawn from the annular volume by other means. FIG. 60 shows a blood separation apparatus 6000 including a sample tube 6010 and a separator float 6030. The sample tube 6010 has a sidewall 6012, a first end 6014 and a second end 6016. The separator float 6030 as depicted includes a main body portion 6032 having a top end 6034 and a bottom end 6036, top support member 6042 and bottom support member 6044 extending radially from the main body portion 6032, and a pressure relief means, such as axial bore 6050 extending from the top end 6034 through the bottom end 6036. An annular volume 6070 is formed between the main body portion 6032 and sidewall 6012.

When the apparatus of FIG. 60 is used to separate buffy coat constituents, at least a portion of the buffy coat constituents is removed from the annular volume 6070 through the sidewall 6012 using a removal device, such as syringe 6080. In this regard, the sidewall is typically formed of a material that is generally sturdy enough to withstand the forces generated by centrifugation, but that can be penetrated by syringe 6080. Preferably, the material can seal the small hole made in the sidewall by the removal device. The criteria for selecting the material include high clarity, injection molding grade, high flow, medium-low modulus, low shrinkage, and cost. In this regard, suitable materials for forming the sample tube 6010 may include acrylics, polyethylene terephthalate glycol (PETG), polycarbonate, polystyrenes, styrene-butadiene-styrene polymers, and TOPAS polymers (amorphous, transparent copolymers based on cyclic olefins and ethylene).

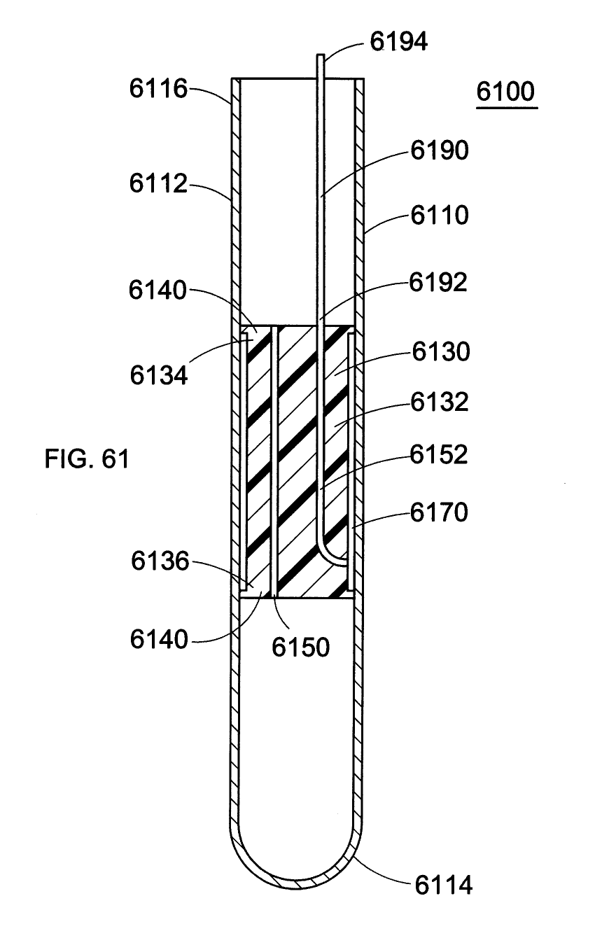

Another concept is illustrated in FIG. 61. Here, a blood separation apparatus 6100 includes a sample tube 6110, a separator float 6130, and a pitot tube 6190. The sample tube 6110 includes a sidewall 6112, a first, closed end 6114, and a second, open end 6116. The separator float 6130 includes a main body portion 6132 having a top end 6134 and a bottom end 6136, and one or more support members 6140 extending radially from the main body portion 6132. A septum 6152 is present in the main body portion 6132, and the septum extends from the top end 6134 to the annular volume 6170. An axial bore 6150 also extends from the top end 6134 through the bottom end 6136.

The pitot tube 6190 has a proximal end 6192 and a distal end 6194. An internal passage 6193 is of course present in the tube, and runs between the proximal and distal ends. The proximal end 6192 engages the septum 6152 at the top end 6134 of the main body portion 6132 and the distal end 6194 is located away from the top end 6134. The separator float 6130 and the pitot tube 6190 may be separate pieces or one integral unit.

When the apparatus of FIG. 61 is used to separate buffy coat constituents, the buffy coat layers/constituents in the annular volume 6170 can be removed through the pitot tube 6190, for example by applying vacuum. In this respect, a pitot tube acts like a straw; fluid flows through the pitot tube when the pressure at the top of the pitot tube is lower than the pressure at the bottom of the pitot tube. When the pitot tube 6190 and septum 6152 are not integral, the pitot tube 6190 engages the septum 6152 prior to removal of the buffy coat layers/constituents.

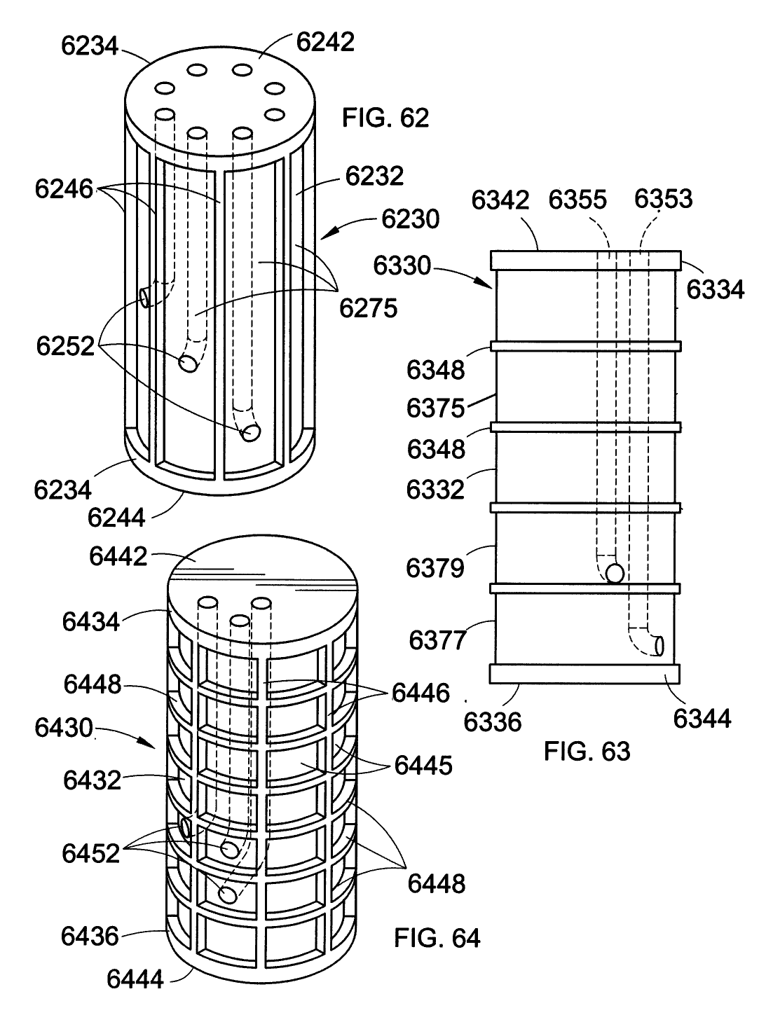

FIGS. 62-64 show different embodiments of a common concept. As seen in FIG. 1, a separator float comprises a main body portion, a top support member, and a bottom support member which define an annular volume in which the buffy coat constituents are trapped. While the float reduces the volume of the blood sample which must be analyzed to locate target cells of interest, it is possible to reduce the volume even further by dividing the annular volume into wells which can be individually accessed. Put another way, the volume within each well can be removed separately from the volume of another well. To accomplish this, the float further includes one or more intermediate support members that form a plurality of wells in the annular volume, i.e. divide the annular volume into a plurality of wells. A plurality of septums is also present within the main body portion, and each septum allows access to, or provides access to, a particular well from the top end of the main body portion. When the buffy coat constituents are removed from a particular well, a fluid, such as air, can be bled into that well to replace the extracted volume. For example, the buffy coat constituents may be drawn out via syringe inserted through the tube sidewall near the bottom of the well while, simultaneously, the sidewall may be pierced at the top of the same well to allow air in to replace the buffy coat constituents. Alternatively, the sample tube adjacent to a particular well may be pierced in two different places to create two ports. Pressure could then be applied to the first port to pump buffy coat constituents out of the second port. A syringe may also be inserted through the float into a well to extract the contents from that well.

In FIG. 62, separator float 6230 includes a main body portion 6232 having a top end 6234 and a bottom end 6236, a top support member 6242 extending radially from the top end 6234, and a bottom support member 6244 extending radially from the bottom end 6236. The intermediate support members 6240 in this embodiment consist of a plurality of axial ridges 6246. The axial ridges extend radially from the main body portion and also extend axially between the top support member 6234 and bottom support member 6244. The axial ridges generally extend radially the same distance from the main body portion as the top support member and the bottom support member. Each axial well 6275 is defined by the main body portion 6232, top support member 6242, bottom support member 6244, and two axial ridges 6246. It is generally contemplated that each axial well 6275 will have the same volume, though this is not a requirement. Each axial well has its own septum 6252, allowing access to the axial well 6275 from the top end 6234 of the main body portion 6232. It may be desirable for the septum to access the axial well near the bottom end 6236 of the main body portion.

In FIG. 63, separator float 6330 includes a main body portion 6332 having a top end 6334 and a bottom end 6336, a top support member 6342 extending radially from the top end 6334, and a bottom support member 6344 extending radially from the bottom end 6336. The intermediate support members in this embodiment consist of a plurality of circumferential ridges 6348. The circumferential ridges extend radially from the main body portion and form a plurality of circumferential wells 6375 in the volume defined by the main body portion 6332, top support member 6342, and bottom support member 6344. Each well 6375 is defined by the main body portion 6332 and at least one circumferential ridge 6348. The circumferential ridges generally extend radially the same distance from the main body portion as the top support member and the bottom support member. It is generally contemplated that each well 6375 will have the same volume, though this is not a requirement. Each well has its own septum 6352, allowing access to the well 6375 from the top end 6334 of the main body portion 6332. In particular embodiments, the septum of each well accesses the well proximal to the support member nearest the bottom end 6336 of the main body portion. For example, septum 6353 accesses well 6377 proximal to bottom support member 6344, while septum 6355 accesses well 6379 proximal to circumferential ridge 6381.

In FIG. 64, separator float 6430 includes a main body portion 6432 having a top end 6434 and a bottom end 6436, a top support member 6442 extending radially from the top end 6434, and a bottom support member 6444 extending radially from the bottom end 6436. The intermediate support members in this embodiment consist of a plurality of axial ridges 6446 and a plurality of circumferential ridges 6448. These ridges 6446, 6448 generally extend radially the same distance from the main body portion as the top support member and the bottom support member. The axial ridges 6446 intersect the circumferential ridges 6448 to form a plurality of wells 6475 in the volume defined by the main body portion 6432, top support member 6442, and bottom support member 6444. It is generally contemplated that each well 6475 will have the same volume, though this is not a requirement. Each well has its own septum 6452, allowing access to a particular well 6475 from the top end 6434 of the main body portion 6432. It may be desirable for the septum to access each well as proximal the bottom end 636 of the main body portion as possible.

When the floats of FIGS. 62-64 are used to separate buffy coat consitutents, the buffy coat layer in a specific well can be extracted using an extraction device such as a syringe or a pitot tube. In particular, the annular volume is first examined to identify the well in which a target cell is located, and only the fluid in that well is extracted for closer analysis.

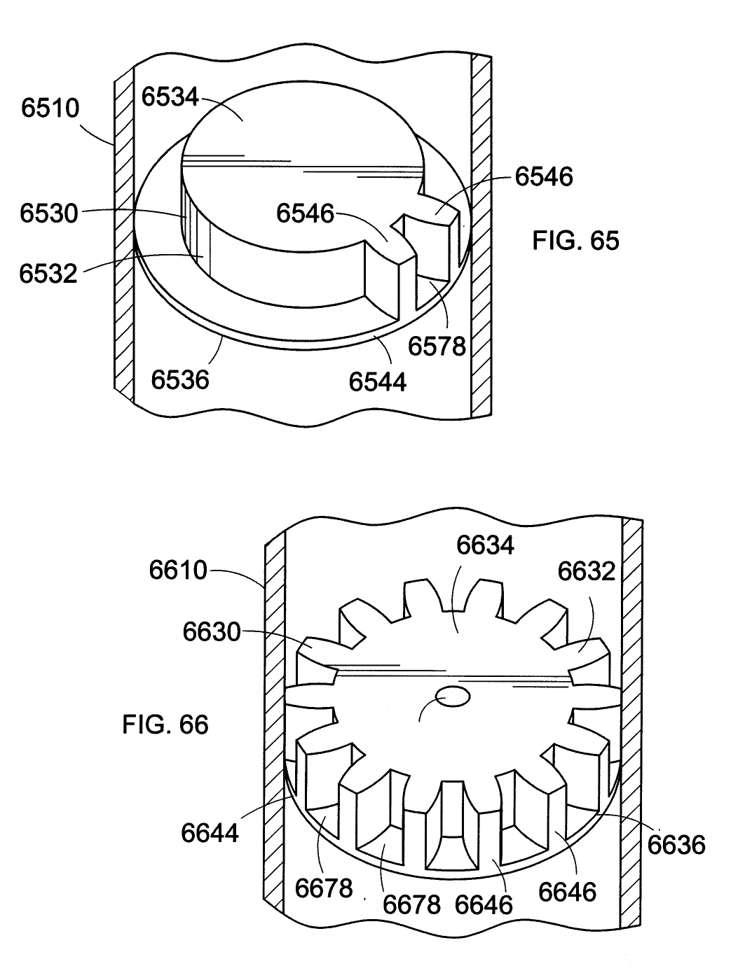

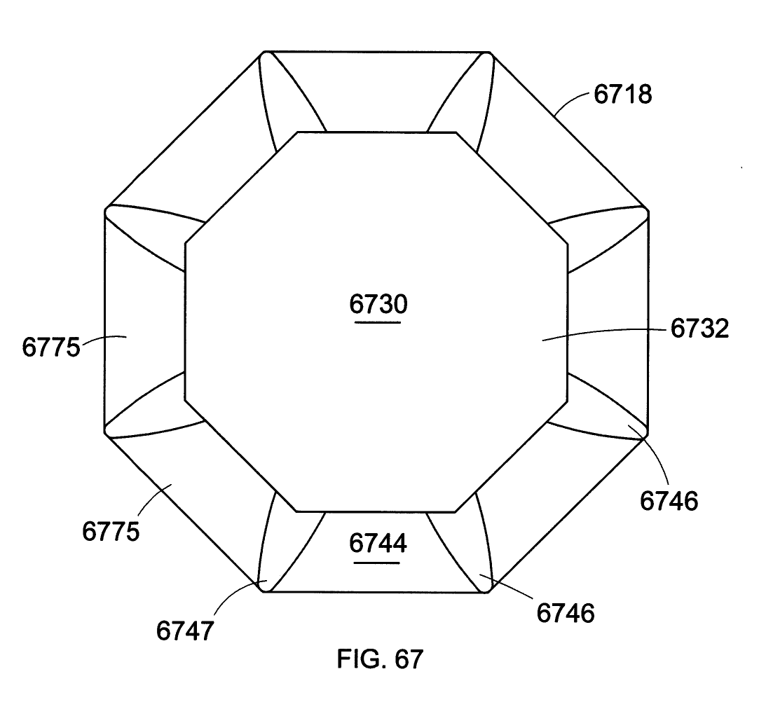

FIG. 65 and FIG. 66 show a related concept. FIG. 65 shows a sample tube 6510 and a separator float 6530. Separator float 6530 includes a main body portion 6532 having a top end 6534 and a bottom end 6536, and a bottom support member 6544 extending radially from the bottom end 6536. A plurality of axial ridges 6546 extend radially from the main body portion 6532 and also extend axially between the top end 6534 and bottom support member 6544. The axial ridges generally extend radially the same distance from the main body portion as the bottom support member. The axial ridges 6546 form an axially extending flute 6578. Here, the liquid in the flute 6578 is accessible from the top end 6534 without the need to include a septum in the main body portion. This may reduce the complexity and cost needed to manufacture the float.

FIG. 66 shows a sample tube 6610 and a separator float 6630. The separator float 6630 includes a main body portion 6632 having a top end 6634 and a bottom end 6636, a bottom support member 6644 extending radially from the bottom end 6636, and a plurality of axial ridges 6646 extending between the top end 6634 and the bottom support member 6644 to form a plurality of flutes 6678 between the axial ridges. An axial bore 6650 is also depicted for relieving pressure differences between the top end 6634 and bottom end 6636.

When the floats of FIG. 65 and FIG. 66 are used, at least a portion of the buffy coat constituents in a specific flute 6578, 6678 is extracted using an extraction device such as a syringe or a pitot tube. In particular, the annular volume is first examined to identify the flute in which a target cell is located, and only the fluid in that flute is extracted for closer analysis.

In additional concepts, a flexible sleeve is used in conjunction with the float. The blood sample and float are placed in the flexible sleeve, which can then be placed into a sample tube. The flexible sleeve itself may be semi-transparent or transparent. After centrifugation, the flexible sleeve is used to seal the buffy coat layers into wells on the float. The sealed wells can then be treated as small slides for analysis.

FIG. 67 shows a top cross-sectional view of a flexible sleeve 6718 and a separator float 6730 exemplifying one concept. The separator float 6730 includes a main body portion 6732 and a plurality of axial ridges 6746. The axial ridges extend radially from the main body portion and also extend axially between the top end (not shown) and the bottom end (not shown) of the main body portion. A bottom support member 6744 is generally present, and a top support member (not seen) may also be present. The ridges generally extend radially the same distance from the main body portion as the bottom support member and the top support member. A plurality of wells 6775 is formed by the ridges. It should be noted that the end 6747 of each ridge 6746 is rounded; this reduces perforation of the flexible sleeve.

The flexible sleeve 6718 generally has a cross-sectional diameter which is less than the diameter of the separator float 6730; this encourages sealing/stretching of the sleeve over the wells 6775. Upon centrifugation, the diameter of the flexible sleeve increases, permitting axial movement of the float so that the float can be aligned with the buffy coat constituents. Upon reducing the rotational speed, the sleeve captures the float. At least one well 6775 is then sealed with the sleeve 6718 to trap a portion of the buffy coat constituents. The sleeve may be held in place by friction, i.e. because of its smaller diameter, or the sleeve can be welded as described above. If no top support member is present, then the buffy coat constituents in a specific well can be removed using a removal device, such as a syringe or a pitot tube, if desired. It should also be noted that if desired, the float can be asymmetrical, i.e. shaped so that the main body portion is not coaxial with the axis of the sample tube or so that different wells have different volumes.

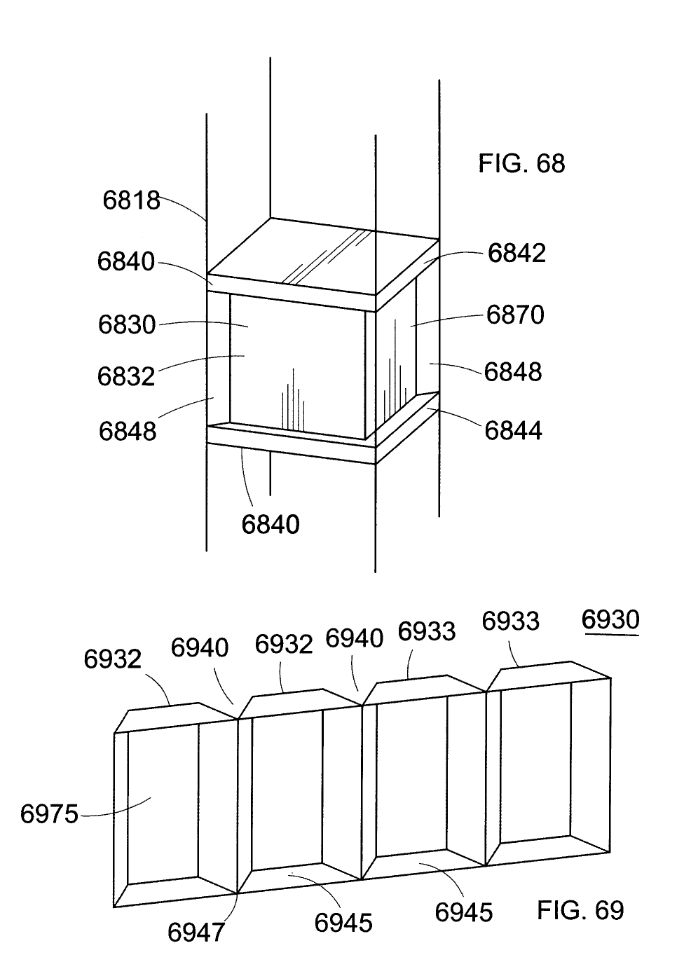

In a related concept, the flexible sleeve has a polygonal cross-sectional shape with n sides, and the float also has n wells. As illustrated in FIG. 68, both flexible sleeve 6818 and a separator float 6830 have a four sided cross-sectional shape. The flexible sleeve has a sidewall 6812 having a four-sided cross-sectional shape. In some embodiments, the lateral cross-section of the sidewall and the float is a regular polygon having n sides. Generally, n is an integer greater than two, and in particular embodiments is three, four, or five (i.e. triangular, square, or pentagon). The float will consequently have n axially-oriented ridges 6848 on corners between the sides to define the wells. It should be noted that again, the wells may be of different volumes. However, generally, all of the wells have the same dimensions and volumes.

The separator float 6830 includes a main body portion 6832 and one or more support members 6840 extending radially from the main body portion 6832. In preferred embodiments, the main body portion 6832 has top support member 6842 extending from top end 6834 and bottom support member 6844 extending from bottom end 6836. Annular volume 6870 is defined by the main body portion 6832 and the sidewall 6812.

Again, the flexible sleeve 6818 generally has a cross-sectional diameter which is less than the diameter of the separator float 6830; this encourages sealing/stretching of the sleeve over the wells 6875. Upon centrifugation, the diameter of the flexible sleeve increases, permitting axial movement of the float so that the float can be aligned with the buffy coat constituents. Upon reducing the rotational speed, the sleeve shrinks and attaches to the float. The wells can be sealed or welded with the sleeve, if desired. Due to the flat surface provided by the sleeve, the wells can then be analyzed like a slide.

In an extension of this concept, the float can be unfolded so that the wells can be analyzed like a stick. FIG. 69 shows a separator float 6930 exemplifying this concept. The separator float 6930 has been unfolded in this depiction. The separator float includes a main body portion 6932 and four axially oriented ridges 6940 extending laterally from the main body portion 6932. A top support member (not shown) and a bottom support member (not shown) also extend laterally from the main body portion. The top support member, bottom support member, and axial ridges generally extend radially the same distance from the main body portion. The float thus defines wells 6975 between the ridges 6940, support members, and main body portion 6932. The main body portion 6932 of the float is adapted to be unfolded so that the exterior surfaces 6945 of the wells can lie substantially in the same plane. Put another way, the main body portion 6932 in this embodiment can be regarded as four different parts 6933, each part providing a surface for each well 6975. The float can be unfolded, for example, by providing thinner material at the end 6947 of each ridge 6940 that is bent, by providing hinges, or other similar methods. Essentially, each ridge acts as a hinge to allow the float to be unfolded. An axial bore can easily be formed by providing that the parts 6933 do not form a solid upon being joined together.

After centrifugation and reduction of the rotational speed, the sleeve shrinks and seals the buffy coat layers/constituents, again by sealing or welding if desired. The float 6930 is unfolded to place the exterior surfaces 6945 of the wells 6975 into substantially the same plane. Another advantage here is that the sleeve may be more easily punctured by a removal device, such as a syringe.

In another set of concepts, the buffy coat constituents are trapped in the annular volume between the sample tube and the float as described above. The buffy coat constituents are then evacuated into a cavity in the main body portion of the float. The buffy coat constituents are then removed from this cavity. If desired, the float containing the buffy coat constituents can be removed from the sample tube, and the buffy coat constituents subsequently removed from the float. Alternatively, the buffy coat constituents can be removed from the float while the float is still in the tube. For example, this concept could be combined with the two-cap sample tube of FIG. 59 to remove the plasma and/or red blood cells prior to accessing the cavity in the float.

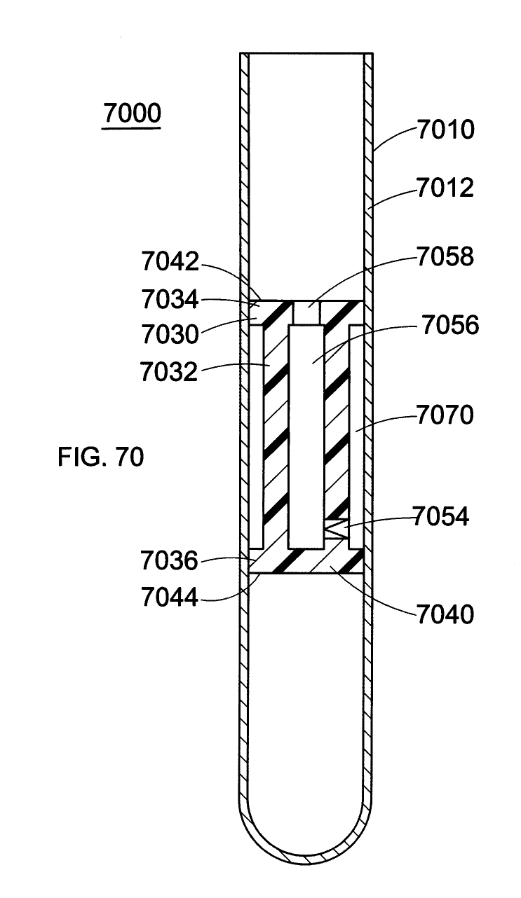

FIG. 70 shows an exemplary embodiment of this concept. Blood separation apparatus 7000 includes a sample tube 7010 and a separator float 7030. The sample tube 7010 includes a sidewall 7012. The separator float includes a main body portion 7032 having a top end 7034, a bottom end 7036. At least one support member 7040 protrudes from the main body portion. The main body portion and the support member 7040 define an annular volume 7070. In particular embodiments, bottom support member 7044 extends radially from the bottom end 7036. In further embodiments, the bottom support member is present, and a top support member 7042 also extends radially from the top end 7034.

A hollow internal cavity 7056 is present within the main body portion. The internal cavity 7056 is connected to the annular volume 7070 by one or more one-way valves 7054 permitting fluid to flow into the hollow internal cavity 7056. Put another way, the one-way valves are oriented to open when the pressure inside the hollow internal cavity is lower than the pressure in the annular volume. In particular embodiments, the one-way valve is proximal to the bottom end 7036 of the main body portion or the bottom support member 7044. A plug 7058, similar to a stopper, may be present at the top end 7034 of the main body portion for accessing the hollow internal cavity 7056. A syringe can be used to penetrate the plug 7058 and access the hollow internal cavity 7056.

The apparatus of FIG. 70 is generally used as described above. During centrifugation, the pressure difference between the annular volume 7070 and the hollow internal cavity 7056 is sufficiently large so as to cause the one-way valve 7054 to open. Buffy coat constituents can then enter the hollow internal cavity 7056 during centrifugation. After centrifugation, the pressure difference is reduced and the one-way valve 7054 closes, trapping buffy coat constituents in the hollow internal cavity 7056. A removal device, such as a pitot tube or syringe, is inserted into the hollow internal cavity 7056 through the plug 7058 to remove buffy coat constituents.

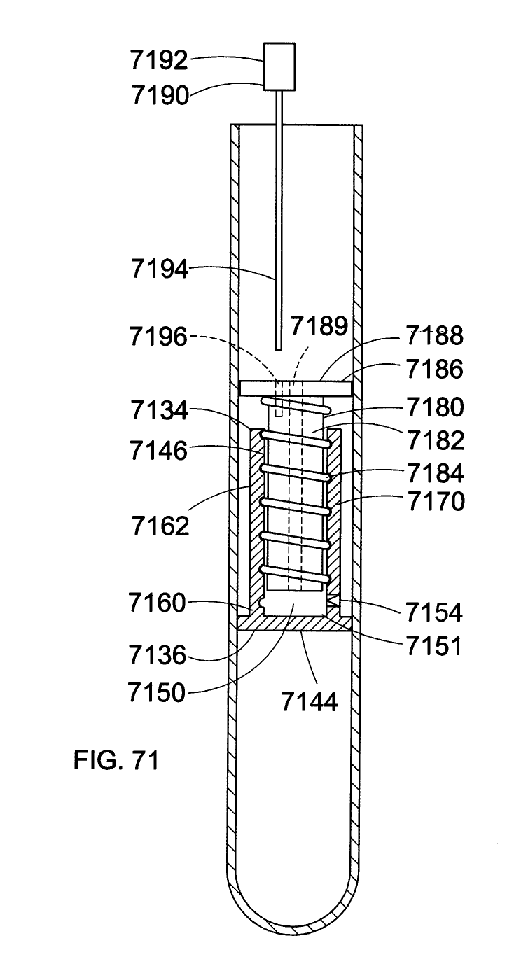

FIG. 71 shows another exemplary embodiment. Separator float 7130 includes a first main body portion 7160 and a second main body portion 7180. The first main body portion 7160 comprises a sidewall 7162 that defines a central bore 7150. The sidewall has a top end 7134 and a bottom end 7136, and the central bore 7150 is accessible from the top end. A bottom support member 7144 extends radially from the bottom end 7136 of the sidewall. A first thread 7146 is located within the central bore. One or more one-way valves 7154 located in the sidewall 7162 permit fluid to flow into the bottom end 7151 of the central bore 7150 from the annular volume 7170. Put another way, the one-way valves are oriented to open when the pressure inside the central bore 7150 is lower than the pressure in the annular volume 7170. The one-way valve is generally located proximal to the bottom support member 7144.

The second main body portion 7180 comprises a center portion 7182 that is sized to fit within the central bore 7150. A complementary thread 7184 is located on the center portion 7182 and engages the first thread 7146. A top support member 7186 extends radially from a top end 7188 of the second main body portion. A plug 7189, similar to a stopper, may be present through the top support member 7186 and the center portion 7182 for accessing the central bore 7150.

The apparatus of FIG. 71 is generally used as described above. In use, the float 7130 is completely threaded so that the central bore 7150 is filled by the center portion 7182. Put another way, the top end 7134 of the first main body portion 7160 contacts the top support member 7186 of the second main body portion 7180. After centrifugation and reduction of the rotational speed, the buffy coat constituents are located in the annular volume 7170. The second main body portion 7180 is then unscrewed from the first main body portion 7160 to increase the internal volume of the central bore 7150. This action reduces the pressure inside the central bore 7150, opening one-way valve 7154 and evacuating the buffy coat constituents into the central bore. A removal device, such as a pitot tube or syringe 7199, can be inserted into the central bore 7150 through the plug 7189 to remove buffy coat constituents. Alternatively, the second main body portion can be partially unscrewed to evacuate the buffy coat constituents. The float is then removed from the sample tube, the second main body portion is completely unscrewed, and the buffy coat constituents can be poured out or otherwise retrieved from the central bore 7150 of the first main body portion.

The second main body portion is unscrewed using a key. As depicted here, the key 7190 comprises a handle 7192 and an interface 7194 that engages a keyhole 7196 present on the top end 7188 of the second main body portion.

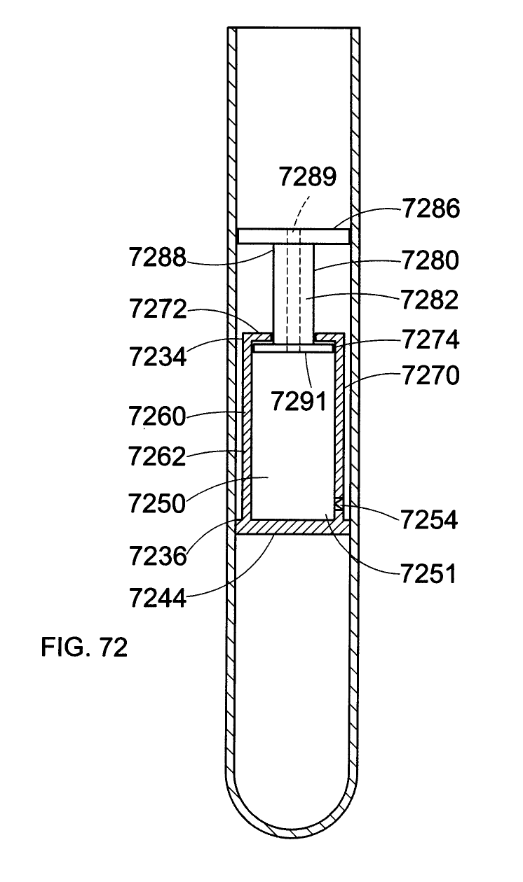

FIG. 72 shows a third exemplary embodiment of a separator float 7230. Separator float 7230 includes a first main body portion 7260 and a second main body portion 7280. The first main body portion 7260 comprises a sidewall 7262 that defines a central bore 7250. The sidewall has a top end 7234 and a bottom end 7236, and the central bore 7250 is accessible from the top end. A bottom support member 7244 extends radially from the bottom end 7236 of the sidewall. One or more one-way valves 7254 located in the sidewall 7262 permit fluid to flow into the bottom end 7251 of the central bore 7250 from the annular volume 7270. Put another way, the one-way valves are oriented to open when the pressure inside the central bore 7250 is lower than the pressure in the annular volume 7270. The one-way valve is generally located proximal to the bottom support member 7244.

The second main body portion 7280 comprises a center portion 7282 that is sized to fit within the central bore 7250. A top support member 7286 extends radially from a top end 7288 of the second main body portion. A plug 7289, similar to a stopper, may be present through the top support member 7286 and the center portion 7282 for accessing the central bore 7250.

The apparatus of FIG. 72 is similar to the apparatus of FIG. 71, but the two main body portions slide apart instead of being unscrewed to increase the internal volume and reduce the pressure in the central bore 7250, thus evacuating buffy coat constituents into the central bore 7250. In this regard, the first main body portion 7260 may include a first lip 7272 at the top end 7234 of sidewall 7262 and the second main body portion 7280 may include a second lip 7274 at the bottom end 7291 of the center portion 7282, the two lips cooperating to form a stop 7276 that ends travel of the main body portions, so the first and second main body portions do not separate.

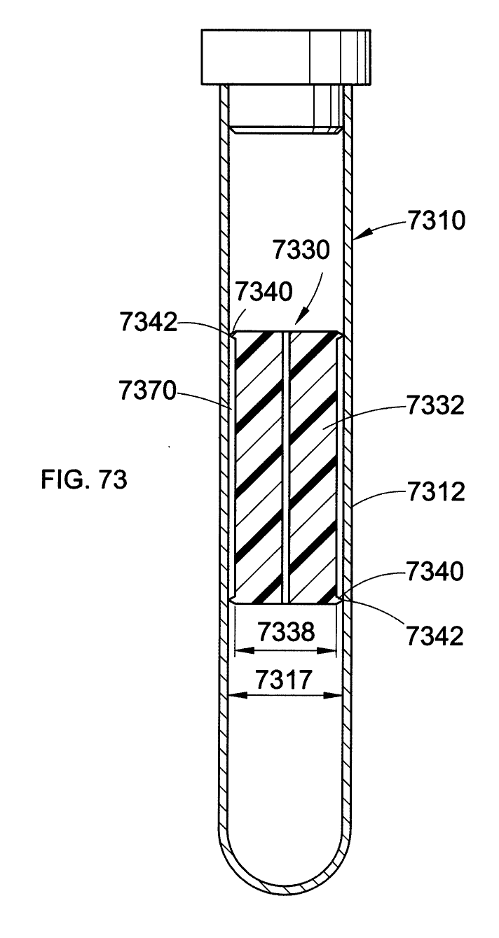

FIG. 73 is another exemplary embodiment of a separator float. The sample tube 7310 is formed from a sidewall 7312. The float 7330 includes a main body portion 7332 and two support members 7340 located at opposite axial ends of the float. The float is sized to have an outer diameter 7317 of the support members 7340 which is greater than the inner diameter 7338 of the main body portion 7332, to form an annular volume 7370. Here, the top and bottom support members 7340 have a sharp circumferential edge 7342. In other words, a pointed perimeter or circumference is provided along the outer diameter 7338 of each support member 7340. After centrifugation, buffy coat constituents are trapped in the annular volume 7370. The sample tube is then compressed against at least one of the top and bottom support members. Under compression, the sharp edge(s) 7342 cut through the tube sidewall 7312, yielding a broken section of the tube containing the float and expanded buffy coat constituents. The tube may be compressed against the float at only the top support member, only the bottom support member, or at both support members. The order in which the tube is compressed against a support member is not believed to be critical. This allows the sample tube to be broken to get access to the float and the buffy coat layers trapped in the annular volume 7370. Of course, the float may also include other intermediate support members, such as the axial ridges or circumferential ridges shown in FIGS. 62-64, helical ridges, or bumps such as those shown in U.S. Pat. No. 7,074,577, the disclosure of which is fully incorporated by reference herein. Such intermediate support members would not have the sharp circumferential edge described in this paragraph.

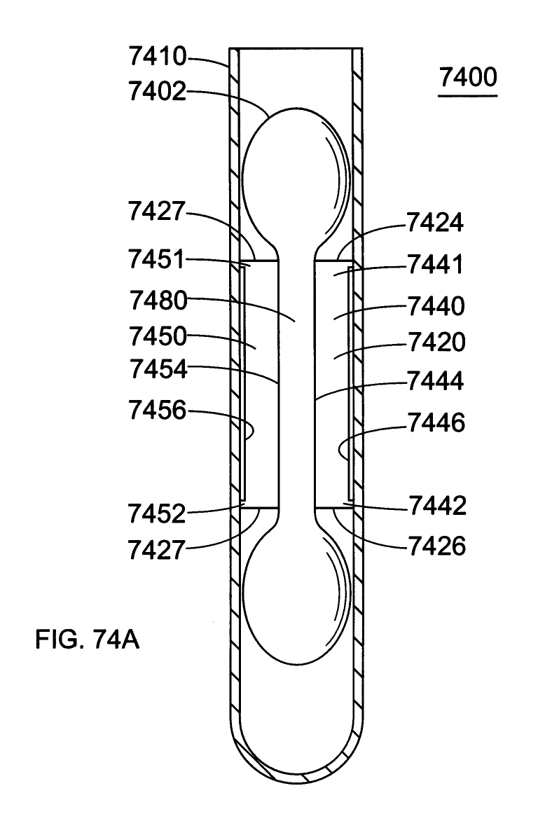

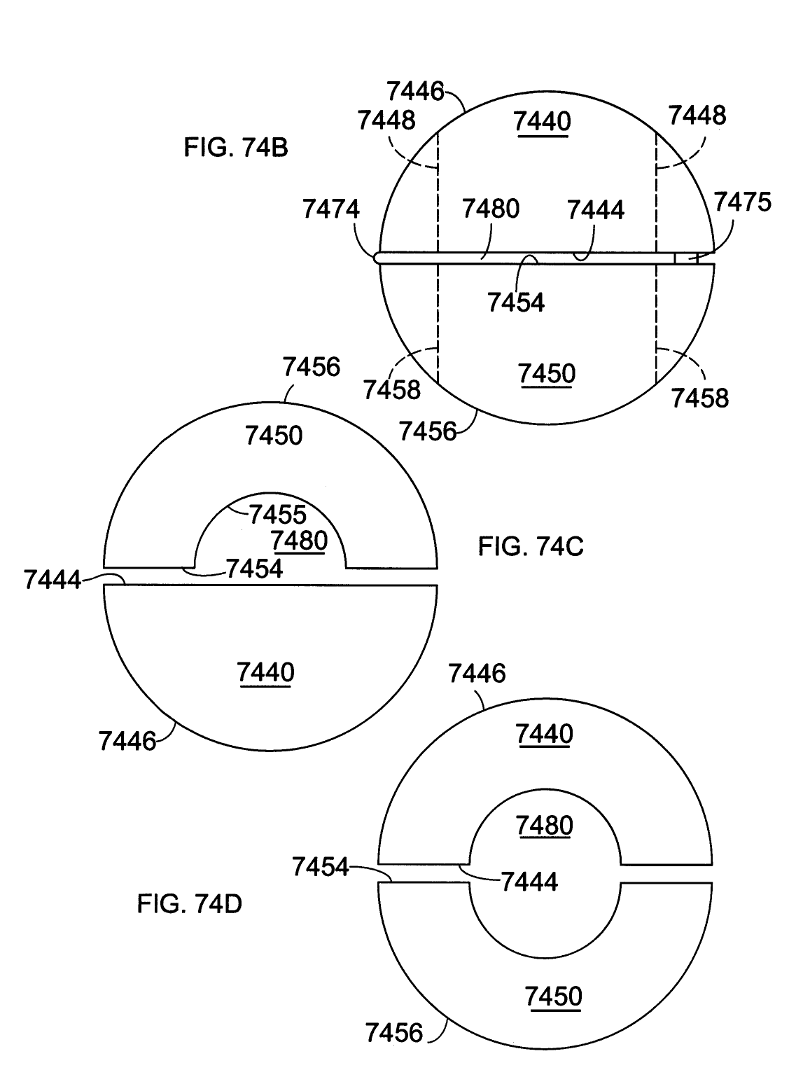

FIGS. 74A-74D illustrate one concept of a blood separation apparatus 7400 where rather than placing the blood sample and float into a sample tube, the blood sample is placed into a flexible bag 7402. FIG. 74A is an axial cross-sectional view. Instead of being placed in the bag and contacting the blood sample, the float 7420 is placed around the bag 7402, i.e. on the exterior of the flexible bag. The flexible bag 7402 and the float 7420 are then placed into a sample tube 7410 and centrifuged. After centrifugation, the buffy coat layers/constituents are in the portion of the bag 7402 located between a first end 7424 of the float and a second end 7426 of the float. The flexible bag 7402 is then sealed at the first end 7424 and the second end 7426 to capture the buffy coat constituents. The sealing may be done, for example, by welding. In particular embodiments, the welding is performed ultrasonically. Ultrasonic welding is an industrial technique commonly used for plastics, whereby high-frequency ultrasonic acoustic vibrations are locally applied to two items being held together to create a solid-state weld between the two items. The term “welding” is used here to indicate the action of closing the bag off in a specific location, and is synonymous with melting.

Separator floats made from two or more pieces are especially suitable for use with this concept. In embodiments, the separator float 7420 comprises a first piece 7440 and a second piece 7450. The first piece 7440 has a first end 7441, a second end 7442, an interior surface 7444, and an exterior surface 7446. In some embodiments, the first piece also includes at least one side surface 7448. The second piece 7450 has a first end 7451, a second end 7452, an interior surface 7454, and an exterior surface 7456. In some embodiments, the second piece also includes at least one side surface 7458. The first piece interior surface 7444 and the second piece interior surface 7454 cooperate to form an open passage 7480 extending between the first end 7424 of the float and the second end 7426 of the float, or in other words between the first end 7441, 7451 and second end 7442, 7452 of each piece 7440, 7450. Each piece 7440, 7450 may also include optional support members 7427 on their exterior surface 7446, 7456 for engaging the sidewall 7412. In some embodiments, the exterior surface of each piece 7440, 7450 comprises at least one support member, and in particular embodiments, the exterior surface of each piece has two support members, i.e. a first support member at the first end and a second support member at the second end.

In particular embodiments, the first and second pieces have substantially the same three-dimensional shape. FIGS. 74B-74D show lateral cross-sectional views of different first and second pieces. In FIG. 74B, the first piece 7440 and second piece 7450 are substantially of the same shape. The interior surface 7444 of the first piece 7440 is substantially planar, i.e. is substantially a straight line in this lateral cross-sectional view. The interior surface 7454 of the second piece 7450 is also substantially planar. The two interior surfaces are substantially parallel to each other, i.e. the open passage 7480 has a rectangular shape. It should be noted that the exterior surface 7446, 7456 is shown contacting the interior surface 7444, 7454 at both ends, and that the exterior surface 7446, 7456 is substantially conforming to an inner surface of the sample tube, for example having a semi-cylindrical surface. In some embodiments, the first piece 7440 and/or second piece 7450 may have at least one side surface 7448, 7458 (shown here as a dotted line). In such embodiments having a side surface, the exterior surface may be described as an arcuate surface, or in a lateral cross-sectional view the exterior surface is an arc. The side surface 7448, 7458 is generally perpendicular to the interior surface 7444, 7454.

The first piece 7440 and second piece 7450 can be connected together using any means. For example, in FIG. 74B, the first piece and second piece are joined on one side by a hinge mechanism 7474, and on the other side by clips 7475. Other connecting mechanisms, such as tongue-and-groove, detent-and-catch, hook-and-loop, etc., can also be used. The connecting mechanism can be located on the interior surface or a side surface.

In FIG. 74C, the interior surface 7444 of the first piece 7440 is substantially planar. The second piece 7450 has a semi-annular cross-sectional shape. Put another way, the interior surface 7454 is substantially a straight line with a central indent. Put yet another way, the interior surface 7454 of the second piece comprises a semi-cylindrical surface 7455.

In FIG. 74D, the first piece 7440 and the second piece 7450 each have a semi-annular cross-sectional shape. The open passage 7480 has a circular shape.

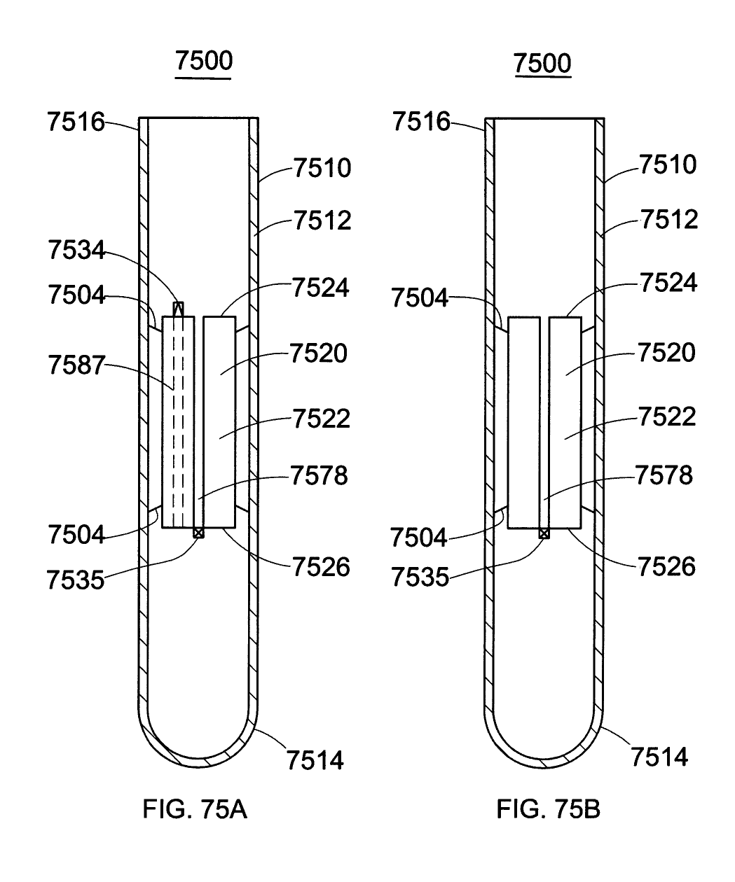

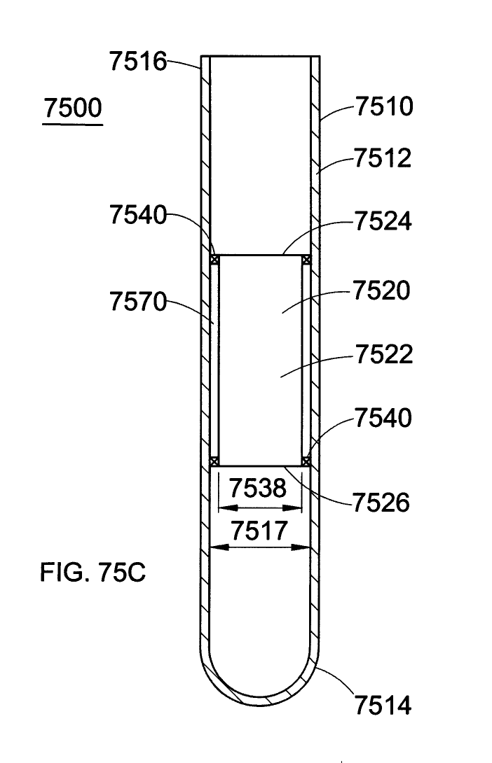

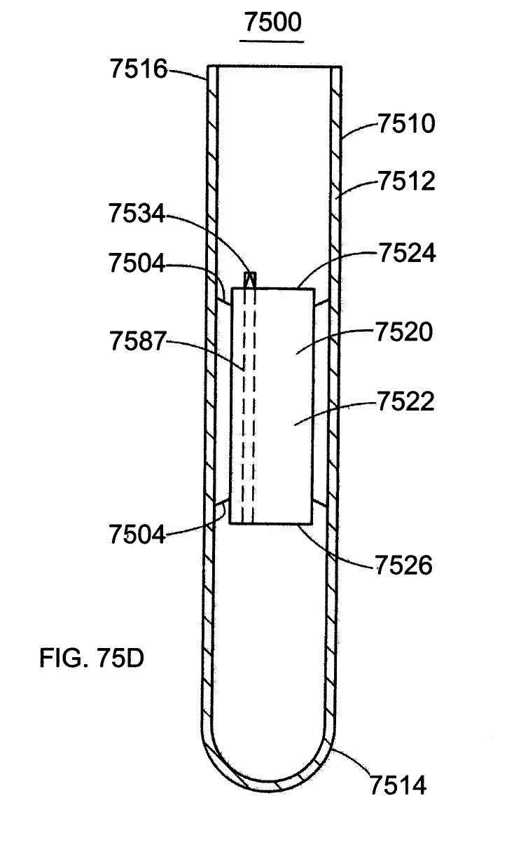

FIG. 75A shows another concept of a blood separation apparatus 7500 including a sample tube 7510 and a separator float 7520. The sample tube 7510 is formed from a sidewall 7512 and has a first, closed end 7514 and a second, open end 7516. The separator float 7520 includes a main body portion 7522 having a first end 7524 and a second end 7526. A pressure relief passage 7587 extends from the second end 7526 to the first end 7524, and has a first one-way pressure relief valve 7534 oriented to open when pressure at the second end 7526 is greater than pressure at the first end 7524 by a specified value. Put another way, the first pressure relief valve 7534 does not open until there is a specified difference between the pressure at the second end 7526 and the pressure at the first end 7524. A buffy coat passage 7578 extends from the second end 7526 to the first end 7524 and has a centrifugation valve 7535 oriented to open during centrifugation. At least one pressure seal wraps around the main body portion. Two pressure seals 7504 are shown here, one extending radially from the first end 7524 and the other from the second end. The pressure seals effectively prevent fluid flow between the main body portion 7522 and the sidewall 7512 of the sample tube.

When the apparatus of FIG. 75A is used, during centrifugation, the centrifugation valve 7535 opens as necessary to allow the blood sample to separate into discrete layers. The centrifugation valve 7535 may be thought of as a weight on a spring. When exposed to higher forces during centrifugation, the valve opens and allows red blood cells, plasma, and buffy coat constituents to flow through the buffy coat passage 7578. As the centrifuge begins to slow down, the valve closes to seal the buffy coat passage. After centrifugation, buffy coat constituents reside in the buffy coat passage 7578. Pressure built up underneath the float 7520 can be relieved through the pressure relief passage 7587 with minimal disturbance to the buffy coat constituents in the buffy coat passage 7578. The float 7520 can be removed from the sample tube 7510 with the buffy coat constituents being present in the buffy coat passage 7578.

FIG. 75B depicts an apparatus similar to the apparatus of FIG. 75A, except that there is no pressure relief passage 7587. It is contemplated here that in the event of a pressure difference between the first end 7524 and the second end 7526, the pressure might cause the float to slide upward along the tube. However, this movement would be acceptable because the centrifugation valve 7535 does not permit the movement of fluid through the buffy coat passage 7587.

FIG. 75C is a cross-sectional view of another exemplary embodiment of a separator float similar to the apparatus of FIG. 75A. Here, the separator float 7520 includes a main body portion 7522 having a first end 7524 and a second end 7526. The main body portion has an outer diameter 7538, while the sample tube 7510 has an inner diameter 7517. Again, there is no pressure relief passage 7587. Rather than pressure seals 7504, at least one support member extends radially outwards from the main body portion towards the sample tube 7510, and has a diameter substantially equivalent to the inner diameter 7517. Here, two support members 7540 are located at the first end 7524 and the second end 7526. The support member(s) can also be described as being circumferentially disposed about the main body portion. The support members are centrifugation valves, as described above. The support member(s) and the main body portion 7522 define an annular volume 7570. The centrifugation valve(s) itself can be considered to have an annular shape when considered in isolation. The centrifugation valves are oriented to allow fluid to flow through the annular volume 7570 from second end 7526 to first end 7524 during centrifugation. When centrifugation ends, the centrifugation valves close, no longer permitting fluid flow through the annular volume. It is again contemplated that any pressure difference between the first end 7524 and the second end 7526 would result only the float sliding upward along the tube.

FIG. 75D is a cross-sectional view of yet another exemplary embodiment of a separator float of the present disclosure. The separator float 7520 is similar to the float of FIG. 75A, except the float 7520 does not include a buffy coat passage 7578 or a centrifugation valve 7535. It is contemplated that the separator float initially begins near the open end 7516 of the sample tube. During centrifugation, the float is pushed downwards into the blood. The resulting pressure on the first one-way pressure relief valve 7534 is sufficient to open the valve, allowing blood components to flow from one end to the other. The various components then settle into layers based on density, with the target cells residing within the pressure relief passage 7587. After centrifugation, the relief valve remains closed, keeping the target cells in the pressure relief passage 7587.

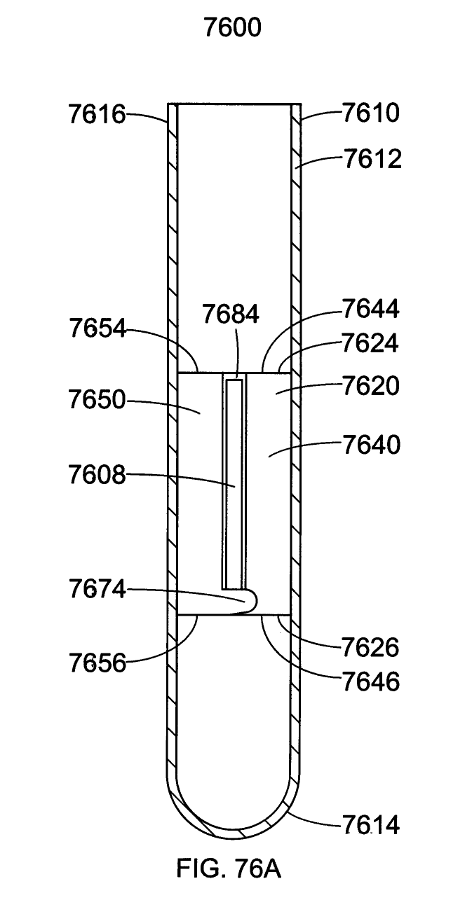

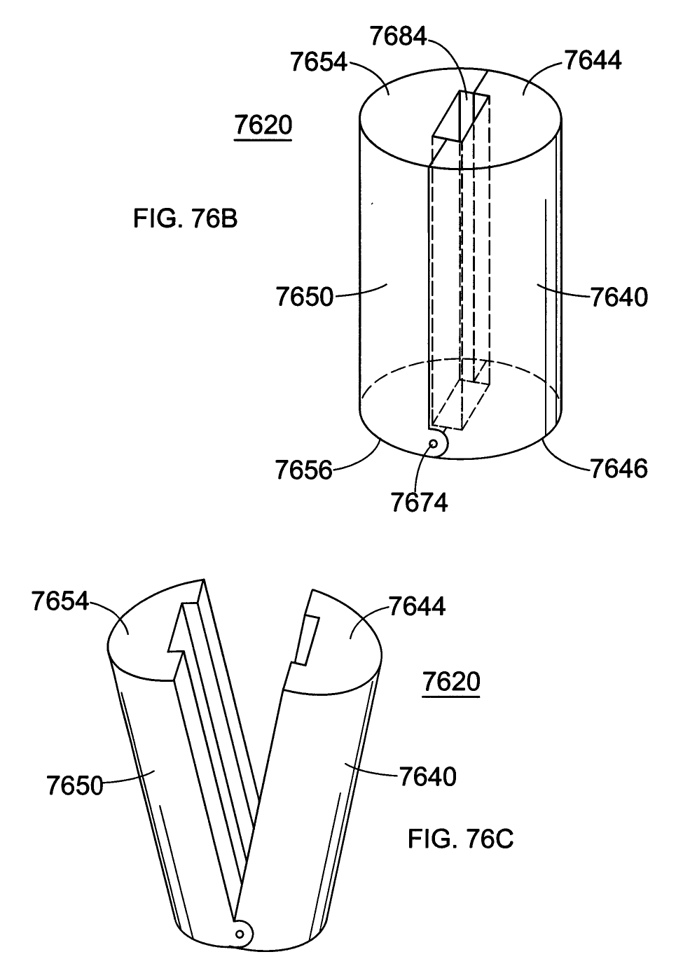

Another concept is illustrated in FIGS. 76A-76C. FIG. 76A is a side view showing a blood separation apparatus 7600 including a sample tube 7610 and a separator float 7620. The sample tube 7610 is formed from a sidewall 7612 and has a first, closed end 7614 and a second, open end 7616.

FIG. 76B is a perspective view of the float in a closed position, and FIG. 76C is a view of the float in an open position. The separator float 7620 is formed from a first piece 7640 and a second piece 7650. The float 7620 has a first end 7626 and a second end 7624. The first piece 7640 has a first end 7646 and a second end 7644. Similarly, the second piece 7650 has a first end 7656 and a second end 7654. The pieces are joined together at their first end 7646, 7656, for example by a hinge 7674. The first piece 7640 and second piece 7650 cooperate to form a rectangular passage 7684 between the first end 7626 and second end 7624 of the float 7620. The float 7620 contains a slide 7608 within the rectangular passage 7684. FIG. 76B shows the float 7620 after removal from the sample tube. FIG. 76C shows the float 7620 after the float 7620 has been opened and the slide has been removed.

In use, the slide 7608 is placed in the float 7620, and the float is centrifugated with the blood sample. Buffy coat constituents present in the rectangular passage 7684 adhere to the slide 7608 within the rectangular passage 7684. After centrifugation, the float 7620 may be removed from the sample tube 7610 and opened at the hinge 7674 to extract the slide 7608. The slide, with the buffy coat constituents already adhered to it, can then be examined.

In other specific embodiments, it is contemplated that the float 7620 can be used with the flexible bag 7402, shown in FIG. 74. In these embodiments, the flexible bag 7402 is placed in the rectangular passage 7684 instead of the slide 7608.

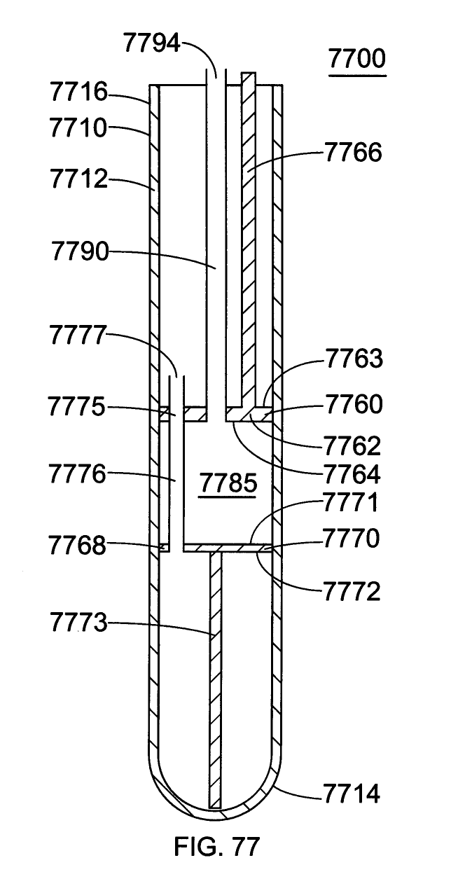

FIG. 77 illustrates still another concept. A side view of a blood separation apparatus 7700 includes a sample tube 7710 and a two-piece float 7720. The sample tube 7710 is formed from a sidewall 7712 and has a first, closed end 7714 and a second, open end 7716.

The two-piece float 7720 includes a top float 7760 and a bottom float 7768. The top float 7760 includes a lower support member 7762 having an upper surface 7763 and a lower surface 7764.

The top float 7760 is formed from a lower support member 7762 and a pitot tube 7790. The lower support member 7762 has an upper surface 7763 and a lower surface 7764. The pitot tube 7790 extends from the lower surface 7764 through the upper surface 7763 and has a top end 7794 located distally from the upper surface 7763. The pitot tube forms a passage from the lower surface 7763 to the top end 7794. In this respect, a pitot tube acts like a straw; fluid flows through the pitot tube when the pressure at the top of the pitot tube is lower than the pressure at the bottom of the pitot tube.

The bottom float 7768 may comprise a support member 7770 having an upper surface 7771 and a lower surface 7772. The top float lower support member lower surface 7764 and the bottom float support member upper surface 7771 are complementarily shaped.

Generally, the density of the top float 7760 and the bottom float 7768 are independently from about 1.029 to about 1.09. The top float 7760 has a density intermediate that of plasma and the buffy coat constituents, or in other words a specific gravity of from about 1.029 to about 1.08. The bottom float 7768 has a density intermediate that of the buffy coat constituents and red blood cells, or in other words a specific gravity of from about 1.08 to about 1.09. Regardless of the value of the density, it is generally contemplated in specific embodiments that the top float has a density which is less than the bottom float.

In use, the apparatus of FIG. 77 traps buffy coat constituents in the volume 7785 between the lower surface 7764 of the lower support member 7762 of the top float 7760 and the upper surface 7771 of the support member 7770 of the bottom float 7768. The top float 7760 is then pushed downwards towards the bottom float 7768 to extract buffy coat constituents through the pitot tube 7790.

In these embodiments, the pitot tube 7790 is integral with the lower support member 7762.

However, it is also contemplated that the pitot tube is made separately from the lower support member. In such embodiments, only the lower support member 7762 of the top float 7760 is centrifuged with the blood sample. The lower support member in this case is made with a first passage from the lower surface 7764 to the upper surface 7763. After centrifugation ends, the pitot tube 7790 is inserted to engage the first passage. The top float 7760 is then pushed down towards the bottom float 7768 to push buffy coat constituents through the pitot tube 7790.

Some additional variations on this two-piece float 7720 are contemplated. The pitot tube 7790 itself is contemplated as being rigid, so as to be suitable for use in pushing the top float 7760 downwards. In some embodiments, however, and as depicted here, the top float 7760 may further comprise a manipulator 7766, such as a handle, extending axially from the upper surface 7763 of the top float 7760, and this manipulator can be used to push the top float downwards. The manipulator is generally made or situated on the upper surface 7763 so that its presence will not affect the final alignment of the lower support member 7762 with the buffy coat constituents.

After centrifugation, excess pressure may form below the bottom float 7768. This pressure may be relieved through a pressure relief tube 7776 which extends axially from the lower surface 7772 of the bottom float support member 7770 through the upper surface 7771 and terminates at an upper end 7777. The top float 7760 includes a second passage 7775 from the lower surface 7764 to the upper surface 7763, and the pressure relief tube 7776 extends through the second passage.

In addition, if desired, an axial support member 7773 may extend axially from the lower surface 7772 of the support member 7770 of the bottom float 7768. It is contemplated that this axial support member 7773 would contact the closed end 7714 of the sample tube 7710 and provide additional resistance when the top float 7760 is pushed towards the bottom float 7768. It should be noted, however, that the length of the axial support member may be uncertain, as the level at which the bottom float support member 7770 rests after centrifugation would depend partially on the size of the blood sample, and the size of the blood sample with which the float is used is not a factor that can be controlled during manufacture of the float.

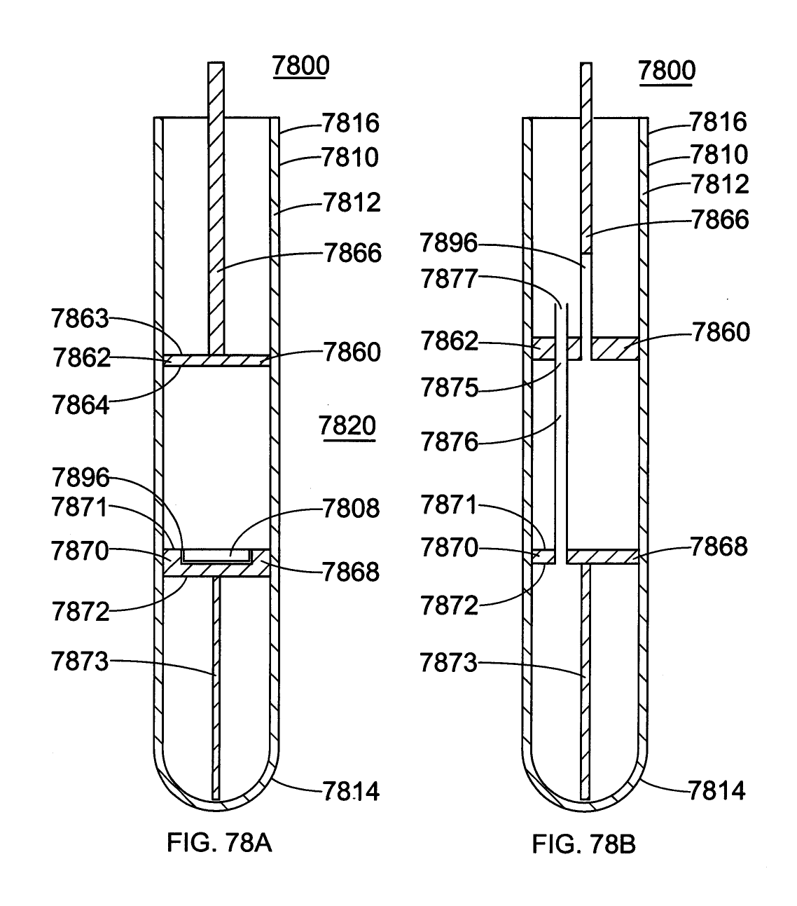

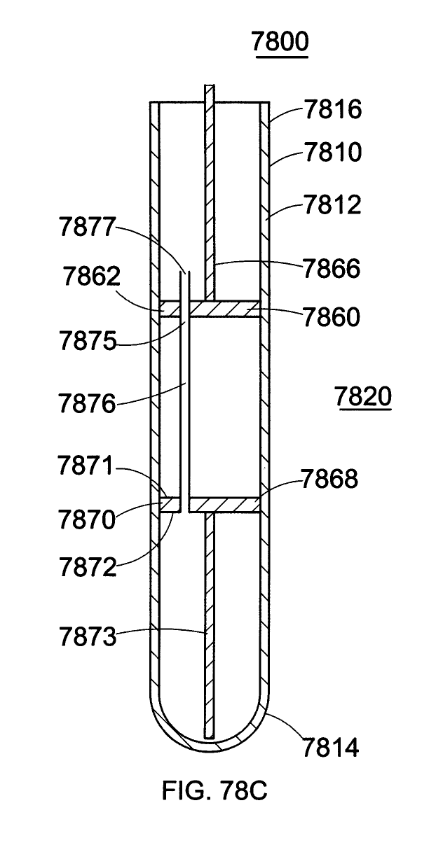

FIGS. 788A-78C show three exemplary embodiments of another concept. Here, a blood separation apparatus 7800 includes a sample tube 7810 and a two piece float 7820. The sample tube 7810 is formed from a sidewall 7812 and has a first, closed end 7814 and a second, open end 7816. Generally speaking, the buffy coat is captured between the two pieces of the float.

The two-piece float can include a recess, in which the buffy coat layer is trapped or contained. The recess is placed in different locations in FIG. 78A and FIG. 78B.

A first exemplary embodiment is shown in FIG. 78A. The two-piece float 7820 comprises a top float 7860 and a bottom float 7868. The top float 7860 includes a lower lateral support member 7862 which has an upper surface 7863 and a lower surface 7864. A member or manipulator 7866 extends axially from the upper surface 7863 of the lower lateral support member 7862. The manipulator 7866 is used to push the top float 7860 towards the bottom float 7868.

The bottom float 7868 comprises an upper lateral support member 7870 having an upper surface 7871 and a lower surface 7872. The top float lower lateral support member 7862 and the bottom float upper lateral support member 7870 are complimentarily shaped to form a recess 7896. For example, FIG. 78A shows five sides of the recess 7896 being formed in the bottom float upper lateral support member 7870, while the top float lower lateral support member 7862 covers the recess. It is contemplated that this arrangement could be reversed, with the top float lower lateral support member 7862 containing the recess and the bottom float upper lateral support member 7870 covering the recess. The recess resides in a lateral plane, in other words perpendicularly to the long axis of the sample tube 7810.

A second exemplary embodiment is shown in FIG. 78B. Here, the recess 7896 is located in the member 7866. The hollow member 7866 is open on the lower surface 7864 of the lower lateral support member 7862, and is adapted to receive the buffy coat layer. The upper lateral support member 7870 of the bottom float 7868 is used to seal the hollow member.

In FIG. 78C, there is no recess. Rather, the buffy coat layers are simply trapped between the top float 7860 and the bottom float 7868.

Generally, the density of the top float 7860 and the bottom float 7868 are independently from about 1.029 to about 1.09. The top float 7860 has a density intermediate that of plasma and the buffy coat constituents, or in other words a specific gravity of from about 1.029 to about 1.08. The bottom float 7868 has a density intermediate that of the buffy coat constituents and red blood cells, or in other words a specific gravity of from about 1.08 to about 1.09. Regardless of the value of the density, it is generally contemplated in specific embodiments that the top float has a density which is less than the bottom float. Preferably, the densities are selected in FIG. 78A and FIG. 78B so that there is little to no additional space between the two floats, so that the buffy coat is located in the recess.

In use, the apparatus of FIGS. 78A-78C traps buffy coat constituents in the volume between the lower surface 7864 of the lower support member 7862 of the top float 7860 and the upper surface 7871 of the support member 7870 of the bottom float 7868. The buffy coat constituents are then removed from the float. In the case of FIG. 78A and FIG. 78B, this may be done by breaking the sample tube to retrieve the float. In the case of FIG. 78C, one end of the sample tube is broken off, and one piece of the two-piece float is then removed so that the buffy coat constituents can be drained out of the remainder of the tube.

When the apparatuses of FIGS. 78A and 78B is used to separate buffy coat constituents, after centrifugation, the buffy coat constituents reside in the volume between the lower surface 7864 of the lower lateral support member 7862 of the top float 7860 and the upper surface 7871 of the upper lateral support member 7870 of the lower float 7868. The member or manipulator 7866 is pushed down to push buffy coat constituents in the recess 7896. The float can then be separated from the sample tube, and the buffy coat can be extracted onto a slide for examination.

In particular embodiments, it is contemplated that a suitable slide 7808 could be placed within the recess 7896, and the buffy coat constituents could adhere to the slide 7808 within the recess 7896. The two-piece float 7820 could then be separated from the sample tube 7810 and the slide 7808 would be removed from the recess 7896 in the two-piece float 7820. The slide, with the buffy coat constituents already adhered to it, could then be examined.

Some additional variations on this two-piece float 7820 are contemplated. After centrifugation, excess pressure may form below the bottom float 7868. This pressure may be relieved through a pressure relief tube 7876 which extends axially from the lower surface 7872 of the bottom float upper lateral support member 7870 through the upper surface 7871 and terminates at an upper end 7877. The top float 7860 includes a second passage 7875 from the lower surface 7864 to the upper surface 7863, and the pressure relief tube 7876 extends through the second passage.

In addition, if desired, an axial support member 7873 may extend axially from the lower surface 7872 of the upper lateral support member 7870 of the bottom float 7868. It is contemplated that this axial support member 7873 would contact the closed end 7814 of the sample tube 7810 and provide additional resistance when the top float 7860 is pushed towards the bottom float 7868. It should be noted, however, that the length of the axial support member may be uncertain, as the level at which the bottom float support member 7870 rests after centrifugation would depend partially on the size of the blood sample, and the size of the blood sample with which the float is used is not a factor that can be controlled during manufacture of the float

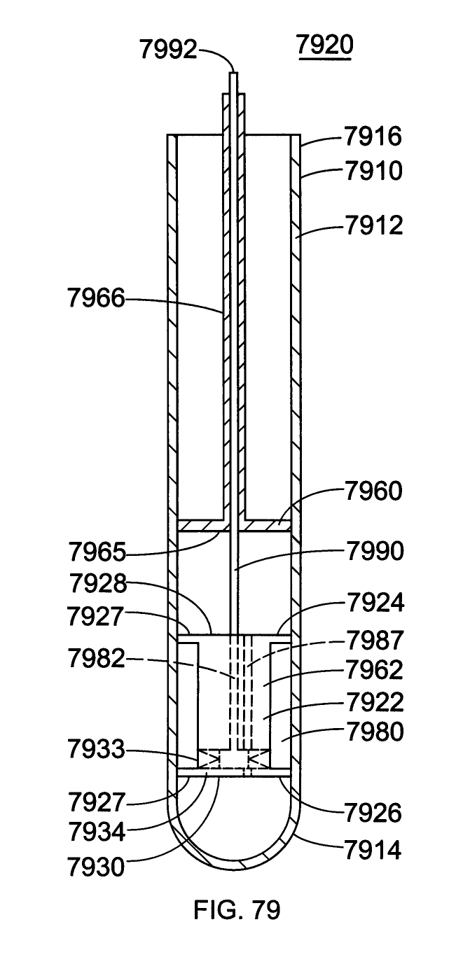

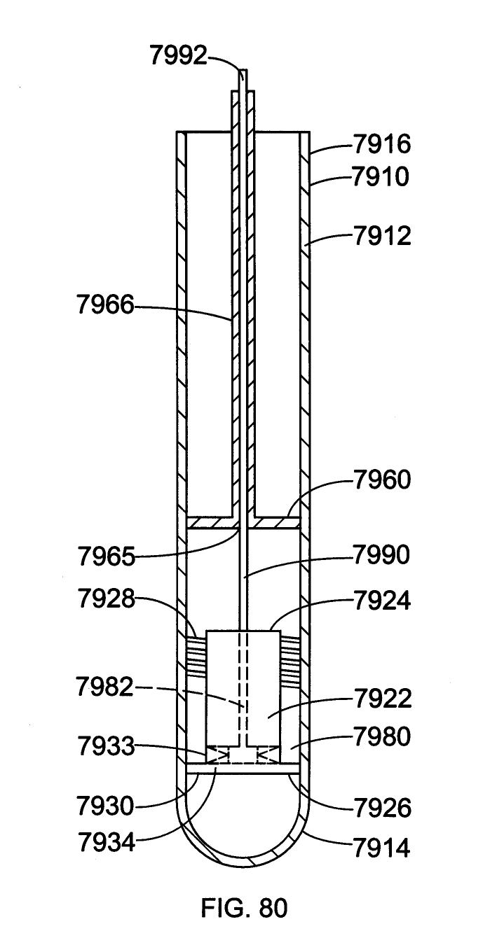

FIG. 79 and FIG. 80 illustrate another concept with two exemplary embodiments. Briefly, the separator is a two-piece float. The upper piece is used to push fluid down towards the lower piece. The lower piece contains a pitot tube through which the buffy coat constituents are extracted from the tube.

FIG. 79 shows a side view of a sample tube 7910 and a separator float 7920. The sample tube 7910 is formed from a sidewall 7912 and has a first, closed end 7914 and a second, open end 7916.

The separator float 7920 includes a lower piece 7962 and an upper piece 7960. The lower piece 7962 is formed from a main body portion 7922. The main body portion 7922 has a top end 7924 and a bottom end 7926. One or more support members 7927 protrude from the main body portion. The main body portion and the support members define an annular volume 7980. A pitot tube 7990 extends axially from the top end 7924 of the main body portion 7922. The main body portion contains an internal passage 7982 that connects a distal end 7992 of the pitot tube 7990 to an opening 7933 in the annular volume 7980. In particular embodiments, the main body portion has a specific gravity of from about 1.029 to about 1.09, including from about 1.08 to about 1.09.

The upper piece 7960 includes a passageway 7965 through which the pitot tube 7990 extends. The upper piece also includes a member 7966 or handle extending axially away from the main body portion 7922. The upper piece 7960 has a lower density than the main body portion 7922, i.e. is less dense. The upper piece and the lower piece both have a diameter sufficient to engage the sidewall 7912 of the sample tube 7910. Put another way, the upper piece 7960 and the lower piece 7962 both have substantially the same diameter.

In some embodiments, the passageway 7965 is located inside the member 7966, i.e. the member is hollow or the member 7966 surrounds the pitot tube 7990. The float 7920 may further include one or more one-way valves 7934 in the opening 7933 of the main body portion which are oriented to permit flow from the annular volume 7980 into the internal passage 7982. Put another way, the one-way valves are oriented to open when the pressure in the annular volume is greater than the pressure in the internal passage. In other embodiments, the internal passage 7982 connects the pitot tube 7990 to a plurality of openings in the annular volume 7980.

When the apparatus of FIG. 79 is used, both the upper piece 7960 and the lower piece 7962 are aligned with each other so that the pitot tube 7990 extends through the passageway 7965, and both pieces are placed into the sample tube with the blood sample prior to centrifugation. During centrifugation, the main body portion 7922 aligns with the buffy coat constituents and traps them in the annular volume 7980 after centrifugation ends. The upper piece 7960, which has a lower density than the main body portion, remains floating and not in contact with the main body portion. The upper piece 7960 is then pushed downwards towards the main body portion to force fluid into the annular volume, which in turn pushes the buffy coat constituents upwards through the pitot tube 7990. The one-way valves 7934 can be used to keep fluid from entering the internal passage 7982, particularly during centrifugation. In these embodiments, the pitot tube 7990 is integral with the main body portion 7922.

However, it is also contemplated that the pitot tube is made separately from the main body portion. In such embodiments, only the main body portion 7922 is centrifuged with the blood sample. The internal passage 7982 connects the opening 7933 in the annular volume 7980 to the top end 7924 of the main body portion. After centrifugation ends, the pitot tube 7990 is inserted to engage the internal passage 7982 at the top end 7924. The upper piece 7960 is then threaded onto the pitot tube, so that the pitot tube extends through the member 7966, and the upper piece 7960 is pushed down towards the main body portion 7922 to force fluid into the annular passage and push buffy coat constituents through the pitot tube 7990. It should be noted that in addition, the upper piece 7960 does not need to have a lower density than the lower piece 7962 because the upper piece 7960 is not centrifuged.

An optional pressure relief passage 7987 may also be present for relieving excessive pressure. The pressure relief passage 7987 is an independent passage between the top end 7924 and the bottom end 7926, and does not intersect the internal passage 7982, any openings 7933, or any one-way valves 7934.

In specific embodiments, the one or more support members 7927 includes a bottom support member 7930 which protrudes from the bottom end 7926 of the main body portion. The opening 7933 may be located proximally to the bottom support member 7930. The one or more support members 7927 may also include a top support member 7928 which protrudes from the top end 7924 of the main body portion, i.e. which is proximal to the top end 7924 of the main body portion.

FIG. 80 is a side view of a second exemplary embodiment similar to FIG. 79. Here, the one or more support members 7927 include a bottom support member 7930 which protrudes from the bottom end 7926 of the main body portion. Here, the top support member 7928 is a helical support member which is proximal to the top end 7924 of the main body portion. One advantage to using a helical support member instead of a flat support member is that when the upper piece 7960 is pushed towards a flat support member as in FIG. 79, fluid is forced into the annular volume 7980 around the entire perimeter of the support member. This can cause mixing of the desired buffy coat constituents with the fluid, increasing the volume which must be extracted through the pitot tube and analyzed later for target cells. However, when the upper piece is pushed towards a helical support member, the fluid enters the annular volume along a smaller periphery and acts like a wave front that “pushes” the buffy coat into the pitot tube. The smaller periphery reduces the volume in which mixing occurs.

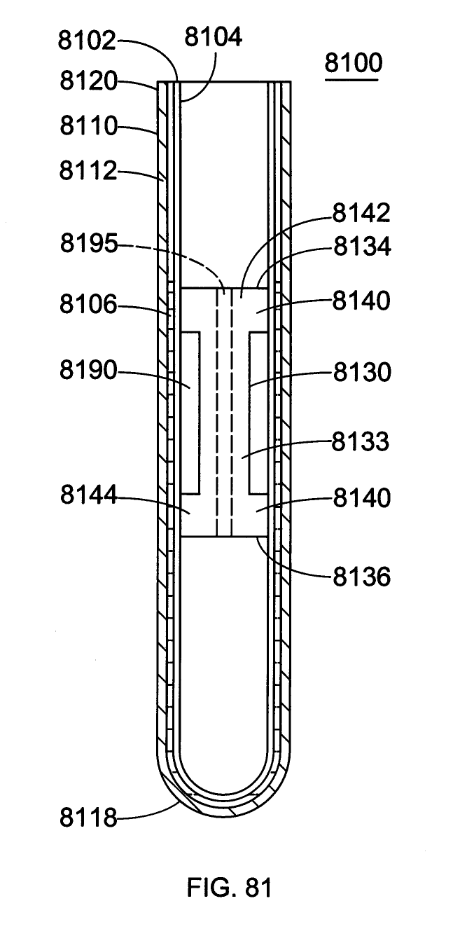

FIG. 81 shows a concept of a blood separation apparatus 8100 including a sample tube 8110, a flexible sleeve 8102, a separator float 8130, and a compressible material 8106 placed between the flexible sleeve 8102 and sample tube 8110. The sample tube 8110 includes a sidewall 8112, a first, closed end 8118, and a second, open end 8120. The flexible sleeve 8102 includes an inner surface 8104 and may be formed of a transparent or semi-transparent material.

The separator float 8130 includes a main body portion 8133 having a top end 8134 and a bottom end 8136. One or more support members 8140 protrude, or extend radially, from the main body portion 8133. The support members 8140 may include a top support member 8142 extending radially from the top end 8134 and a bottom support member 8144 extending radially from the bottom end 8136. A pressure relief means, such as an axial bore 8195, can extend from the top end 8134 through the bottom end 8136 to relieve excessive pressure below the float. The main body portion 8133 and the inner surface 8104 of the flexible sleeve 8102 define an annular volume 8190.

Prior to centrifugation, the compressible material 8106 is between the flexible sleeve 8102 and sample tube 8110. The compressible material applies pressure to the sleeve, causing the inner surface 8104 of the flexible sleeve 8102 to engage the float 8130. The compressible material is usually present in a volume such that the level of the compressible material is above the level of the float, prior to centrifugation. The compressible material 8106 may be water, a slurry, a gel, a foam, or an elastomer. Desirably, the compressible material has a viscosity low enough so that it does not adhere to the sleeve 8102.

Prior to centrifugation, the blood sample and the float 8130 are introduced into the flexible sleeve 8102; the compressible material 8106 is fed to the sample tube 8110; and the flexible sleeve 8102 is placed into the sample tube 8110. The steps of introducing the sample to the sleeve 8102, introducing the float 8130 to the sleeve 8102, feeding compressible material 8106 to the sample tube 8110, and placing the sleeve 8102 into the sample tube 8110 can generally be performed in any order. However, the blood sample and the float are generally introduced into the flexible sleeve prior to placing the flexible sleeve into the sample tube.

During centrifugation, the compressible material 8106 is compressed or moved, so that the pressure on the flexible sleeve 8102 is reduced. This reduction releases the float 8130, allowing the float to align with the buffy coat constituents. When the rotational speed is reduced, the compressible material 8106 returns to its original position or shape, thus applying pressure again and causing the flexible sleeve 8102 to engage the float 8130 and trap the buffy coat constituents in the annular volume 8190. The flexible sleeve 8102 can then be removed from the sample tube 8110 and the blood sample present in the annular volume 8190 can be analyzed. In this regard, desirably the compressible material has a low viscosity, so that the compressible material can drip or otherwise easily be removed from the flexible sleeve to prevent any difficulties in analyzing the blood sample.

In some embodiments, at least one support member 8140 is welded to the flexible sleeve 8102. In particular embodiments, a top support member and a bottom support member are welded to the flexible sleeve. The welding may be performed ultrasonically (i.e. by ultrasonic welding). Again, the compressible material 8106 is generally fed into the sample tube 8110 in a volume such that the compressible material 8106 is at a level in the sample tube 8110 higher than the top end 8134 or the main body portion 8133 of the float 8130 after centrifugation.

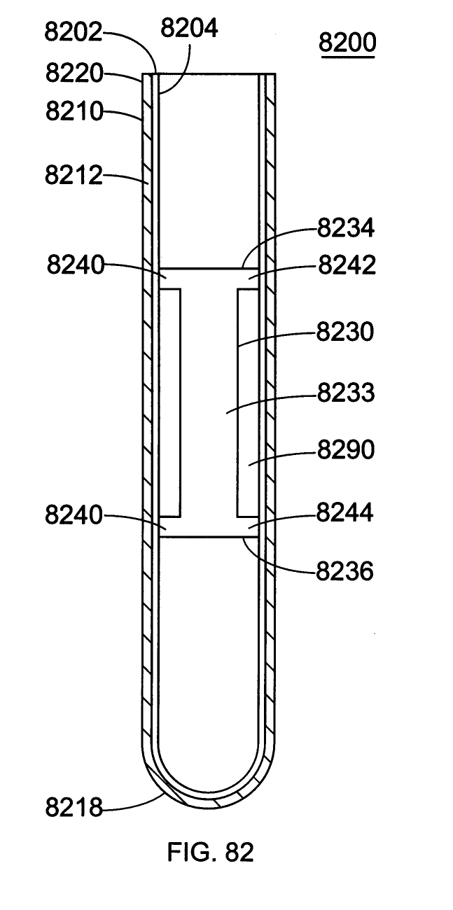

FIG. 82 shows another concept of an apparatus 8200 for separating blood samples. The apparatus 8200 includes a metal sample tube 8210, a flexible sleeve 8202, and a separator float 8230. The sample tube 8210 includes a sidewall 8212, a first, closed end 8218, and a second, open end 8220. The flexible sleeve 8202 includes an inner surface 8204 and may be formed of a transparent or semi-transparent material.

The separator float 8230 includes a main body portion 8233 having a top end 8234 and a bottom end 8236. One or more support members 8240 protrude, or extend radially from the main body portion 8233. The support members 8240 may include a top support member 8242 extending radially from the top end 8234 and a bottom support member 8244 extending radially from the bottom end 8236. A pressure relief means may be present, such as an axial bore (not shown) extending from the top end 8234 through the bottom end 8236. The main body portion 8233 and the inner surface 8204 of the flexible sleeve 8202 define an annular volume 8290.

This apparatus is used as generally described above, with the blood sample and the float being introduced into the flexible sleeve, the flexible sleeve being placed into the metal sample tube, and centrifugation being applied to align the float (and the annular volume 8290) with the buffy coat constituents. As centrifugation ends and the rotational speed is being reduced, here, the metal tube 8210 is constricted or crushed. This causes the metal tube to capture the sleeve 8202 and the float 8230, trapping the buffy coat constituents in the annular volume 8290. The tube 8210 may be constricted before, during, or after the reduction of the rotational speed.

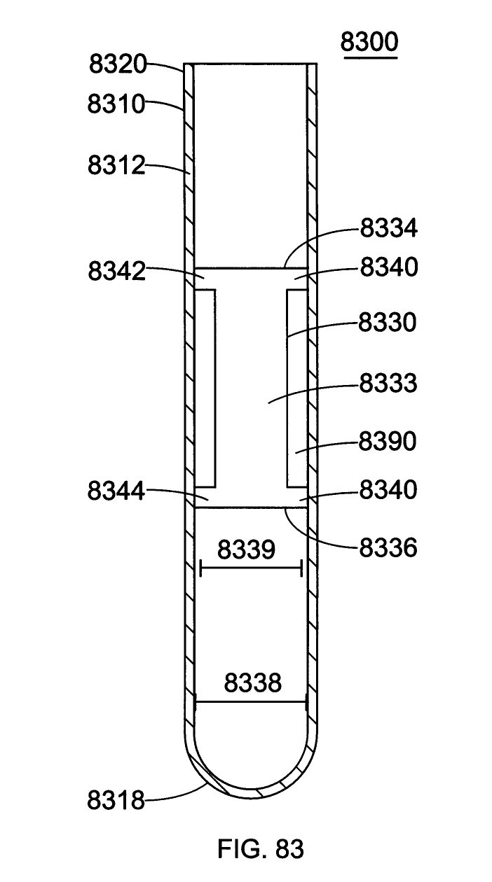

FIG. 83 illustrates an apparatus 8300 where the separator float 8330 is flexible, instead of the sample tube. The sample tube 8310 includes a sidewall 8312, a first, closed end 8318, and a second, open end 8320. The sidewall of the sample tube can be rigid or flexible.

The separator float 8330 includes a main body portion 8333 having a top end 8334 and a bottom end 8336. One or more support members 8340 protrude, or extend radially, from the main body portion 8333. The support members 8340 may include a top support member 8342 extending radially from the top end 8334 and a bottom support member 8344 extending radially from the bottom end 8336. The main body portion 8333 and the sidewall 8312 of the sample tube 8310 together define an annular volume 8390. The float 8330 optionally includes a pressure relief means, such as an axial bore (not shown).

When the float is not under centrifugal pressure, the float 8330 has a first cross-sectional diameter 8338. However, during centrifugation, the diameter of the float 8330 shrinks to a second cross-sectional diameter 8339 which is less than the first cross-sectional diameter 8338 due to the centrifugal force. The second cross-sectional diameter 8339 is sufficiently small that the float can move within the sample tube 8310. This change in diameter permits the float 8330 to align with the buffy coat constituents. The centrifugal force is dependent upon the pressure created during centrifugation—the lower the speed, the lower the centrifugal force generated. The float is generally designed to collapse at a relatively low force, and the degree to which the diameter decreases should be limited. When the rotational speed is reduced, the float 8330 enlarges to the first cross-sectional diameter 8338, trapping the buffy coat constituents in the annular volume 8390.

The flexible float 8330 includes flexible and/or compressible materials. However, the entire float does not need to be made of such materials. For example, the main body portion 8333 may be made from rigid materials, while the support members 8340 are made from a compressible material, or vice versa. In some embodiments, however, the main body portion 8333 and the support members 8340 are made from compressible materials. Suitable flexible and/or compressible materials may include flexible polymers. Exemplary flexible polymers includes urethane, rubber, and silicone polymers.

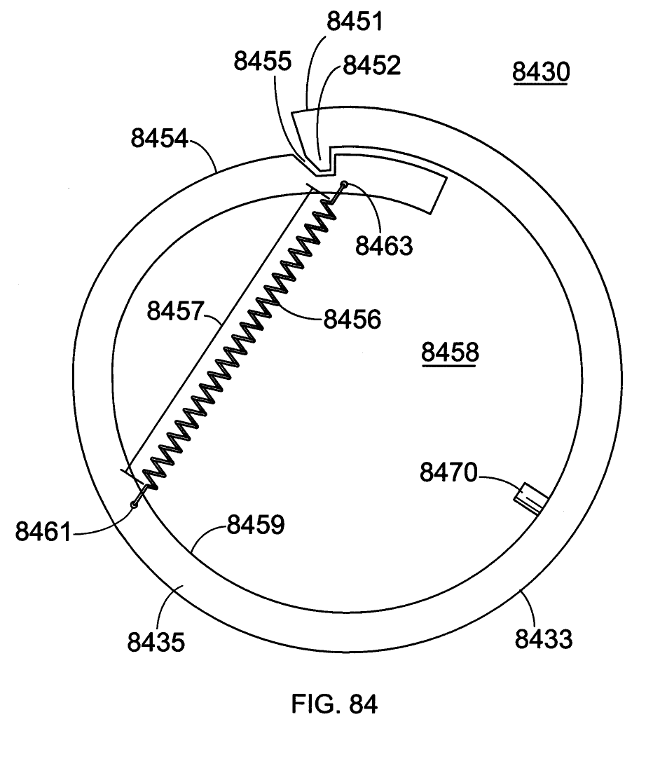

FIG. 84 shows a top, cross-sectional view of another embodiment of a flexible separator float 8430. The separator float 8430 includes a main body portion 8433 which is formed from a flexible sidewall 8435. The flexible sidewall 8435 has a first edge 8451 and a second edge 8454. The term “edge” is used here to refer to an area or volume along one side of the sidewall, and not in the mathematical sense of a one-dimensional line. The first edge 8451 and second edge 8454 overlap to define an interior volume 8458. An interior surface 8459 is present within the interior volume 8458.

A detent 8452 is present along the first edge 8451. The detent 8452 engages a notch 8455 which is present along the second edge 8454. A spring 8456 is also present within the interior volume 8458. The spring has a first end 8461 and a second end 8463. The first end 8461 of the spring is attached to the interior surface 8459 and the second end 8463 of the spring is attached to the second edge 8454 of the flexible sidewall 8435. Put another way, the spring 8456 connects the interior surface 8459 to the second edge 8454. The spring 8456 is constructed so that at rest, the spring has a given length 8457 and can be compressed to a shorter length. The flexible sidewall 8435 may be considered as a sheet that is under some tension and desires to unfold/unroll itself. This bias ensures that the detent 8452 engages the notch 8455 as a default position. It should be noted that FIG. 84 is a cross-sectional view. The flexible sidewall 8435 may be made so that the detent 8452 and notch 8455 are along the entire axial length, or along only a portion of the axial length of the sidewall 8435. There may be more than one spring present as well.

In some embodiments, the float 8430 may further include one or more support members protruding radially from the main body portion 8433 and engaging the sidewall of the sample tube 8410. In particular embodiments, a top support member (not visible) protrudes from the top end 8434 and a bottom support member (not visible) protrudes from the bottom end (not visible) of the main body portion 8433. The main body portion 8433, support members 8440, and tube sidewall define an annular volume 8490. The two ends of the float are sealed and will also reduce in diameter, for example by flexing of the float. In particular embodiments, it is contemplated that the top end and the bottom end of the float are formed from a surface that has a conical shape, the base of the cone forming the top end or bottom end, and the apex of the cone being contained inside the float.

Prior to centrifugation, the detent 8452 on the first edge 8451 engages a notch 8455 which is present along the second edge 8454. When engaged, the flexible sidewall is prevented from further expansion. During centrifugation, the spring 8456 is compressed by the centrifugal force. As the spring shortens, the spring pulls the second edge 8454. This pulling action detaches the detent 8452 from the notch 8455, permitting the detent to travel along the sidewall 8435, thereby reducing the diameter of the float 8430. The reduction in diameter permits the float 8430 to move into alignment with the buffy coat constituents. If desired, a stop 8470 may be present to limit the travel of the second edge 8454 and prevent the float from being damaged due to overstressing the flexible sidewall 8435 during centrifugation. When the rotational speed is reduced, the spring 8456 expands and the diameter of the float 8430 increases until the detent 8452 again engages the notch 8455. The expansion of the float traps the buffy coat constituents in the annular volume 8490.

It is also contemplated that the float could be placed into the sample tube in its reduced diameter. Centrifugation releases the detent, but the centrifugal force keeps the diameter of the float in its reduced state, i.e. smaller than the internal diameter of the sample tube due to the centrifugal force. After centrifugation ends, the diameter of the float would then expand up to the internal diameter of the tube. Again, the sample tube may be either rigid or flexible.

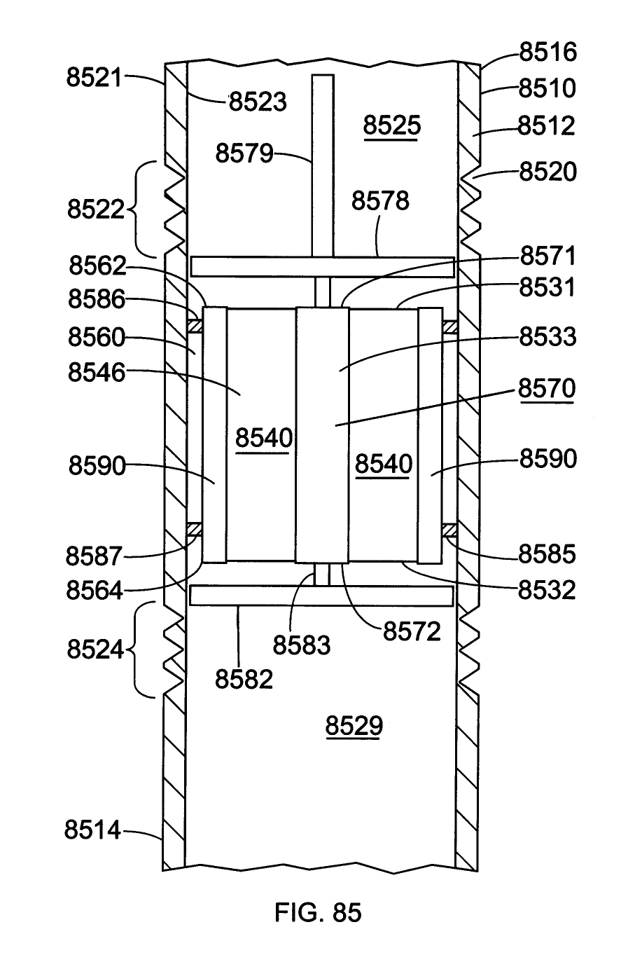

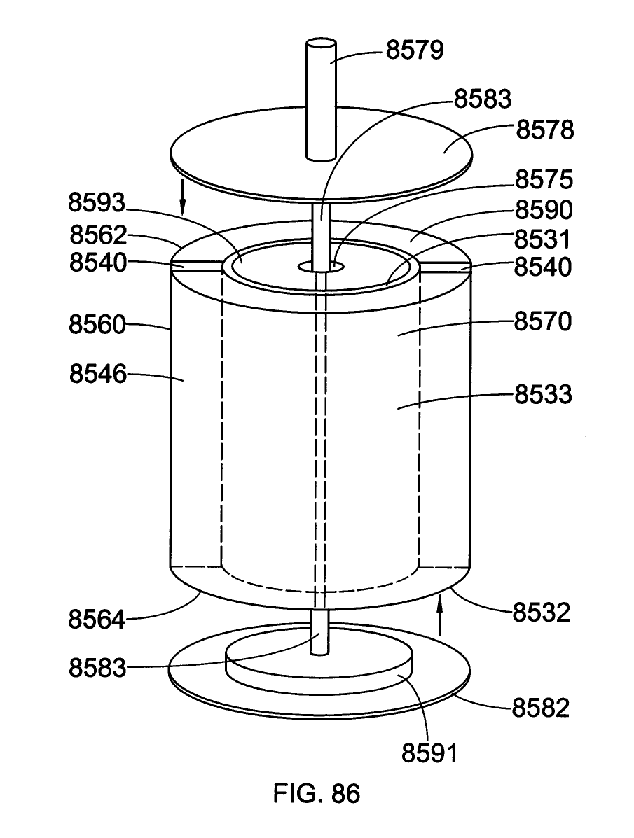

FIG. 85 and FIG. 86 illustrate another concept for a sample tube 8510 and separator float 8530. FIG. 85 is a side view, while FIG. 86 is a perspective view. The sample tube 8510 includes a sidewall 8512, a first, closed end 8514 (closed portion not shown), and a second, open end 8516.

The main body portion 8533 of the separator float 8530 has a top end 8531 and a bottom end 8532. The main body portion is constructed from an inner core 8570 and an optically clear outer sidewall 8560. The inner core 8570 has a top end 8571 and a bottom end 8572. The optically clear outer sidewall 8560 also has a top end 8562 and a bottom end 8564. It is contemplated that the inner core 8570 and the outer sidewall 8560 generally have the same axial length, as shown in FIG. 86. However, in some embodiments, as depicted in FIG. 85, the inner core 8570 has a longer length than the outer sidewall 8560, i.e. the inner core 8570 is longer than the outer sidewall 8560.

At least one support member 8540 extends radially and connects the inner core 8570 to the outer sidewall 8560. Three support members are visible in FIG. 86. As shown there, the support members may comprise a plurality of axial ridges 8546 extending axially from the top end 8571 to the bottom end 8572 of the inner core 8570. An annular volume 8590 is defined between the inner core 8570 and the outer sidewall 8560.

In some embodiments, at least one high pressure seal 8585 surrounds the outer sidewall 8560. As shown here, a top high pressure seal 8586 is present around the top end 8562 of the outer sidewall 8560 and a bottom low pressure seal 8587 is present around the bottom end 8564 of the outer sidewall 8560. The pressure seal(s) effectively prevent(s) fluid flow between the outer sidewall 8560 and the sidewall 8512 of the sample tube. An optional pressure relief passage (not shown) may extend axially from the top end 8571 of the inner core 8570 to the bottom end 8572.

The separator float 8530 is used as generally described above. In particular, when the main body portion 8533 is captured after centrifugation, buffy coat constituents reside in the annular volume 8590. The buffy coat constituents may then be analyzed through the optically clear outer sidewall 8560.

The outer sidewall may be considered “optically clear” if images of the cells in the buffy coat constituents can be taken through the sidewall. In some embodiments, the outer sidewall has a transparency (% T) of more than 90%, or a haze level of 5% or below, when measured according to ASTM D1003.

It may be desirable to be able to retrieve the buffy coat constituents from the sample tube. In some embodiments, the float 8530 may further comprise a bottom end cap 8582 used for sealing the bottom end 8532 of the float 8530. The bottom end cap 8582 may have a diameter substantially equal to the diameter of the outer sidewall 8560. The bottom end cap 8582 and bottom end 8572 of the inner core 8570 may comprise a mutual engagement system for connecting the bottom end cap 8582 to the inner core 8570. Suitable engagement systems include tongue-and-groove, detent-and-catch, hook-and-loop, etc. Alternatively, the cap could be welded to the bottom end 8564 of the outer sidewall 8560.

Similarly, the float 8530 may also further comprise a top end cap 8578 for sealing the top end 8531 of the float 8530. The top end cap 8578 may have a diameter substantially equal to the diameter of the outer sidewall 8560. The top end cap 8578 and top end 8571 of the inner core 8570 may comprise a mutual engagement system for connecting the top end cap 8578 to the inner core 8570.

In FIG. 86, the bottom end cap 8582 is shown as having a tongue 8591 that can be inserted into a groove (not visible), and the top end cap 8578 has a tongue (not visible) that can be inserted into a groove 8593 in the top end 8571 of the inner core 8570. This illustrates one type of mutual engagement system which could be used for sealing the float. The top end cap could also be welded to the top end 8562 of the outer sidewall 8560. In particular embodiments, the float comprises both the bottom end cap and the top end cap.

The top end cap 8578 generally comprises a top end cap member or handle 8579 extending axially away from the float 8530. The bottom end cap 8582 also generally comprises a bottom end cap manipulator or handle 8583 extending axially through an internal passage 8575 in the inner core 8570. The internal passage 8575 extends from the bottom end 8572 through the top end 8571. In some embodiments, the top end cap member 8579 is hollow and the bottom end cap manipulator 8583 extends therethrough. The handles 8579, 8583 may be integral to the respective caps, or separate pieces that can be engaged with their respective cap after centrifugation ends. Pushing the top end cap member 8579 engages the top end cap 8578 with the top end 8571 of the inner core. Pulling the bottom end cap manipulator 8583 engages the bottom end cap 8582 with the bottom end 8572 of the inner core.

When the bottom end cap or top end cap are used with the float 8530, they should be inserted into the sample tube 8510 so that the bottom end cap 8582 is closer to the first end 8514 of the sample tube than the main body portion 8533 and the top end cap 8578 is closer to the second end 8516 of the sample tube than the main body portion 8533. The caps should not hinder flow through the main body portion 8533 during centrifugation. This goal can be achieved by making the bottom end cap 8582 slightly denser than the main body portion 8533, and by making the top end cap 8578 slightly less dense than the main body portion 8533. In embodiments, the bottom end cap 8582 has a specific gravity of greater than about 1.09. In embodiments, the top end cap 8578 has a specific gravity of less than about 1.08. However, the caps should not become located too far away from the main body portion 8533 because any volume between a cap and the main body portion at the end of centrifugation may also become sealed in the annular volume 8590, which would increase the pressure therein and possibly cause one of the caps to fail.

The float 8530 is desirably used in combination with a sample tube 8510 from which the float can be extracted or removed. The sample tube 8510 includes a sidewall 8512, a first, closed end 8514, a second, open end 8516, and circumferential notches 8520. A circumferential notch is formed by one or more grooves that lie substantially within the same plane, that plane being perpendicular to the sidewall of the tube. A first set 8522 of notches is located above the float 8530 and a second set 8524 of notches is located below the float. Each set is shown here in FIG. 85 with three notches, but this number can vary and is generally between one and four notches in each set. The sample tube is broken along one or more notches to get access to the float and the buffy coat layers trapped in the annular volume 8590. See the discussion of FIGS. 58A-58E in this regard.

The present disclosure has been described with reference to exemplary embodiments. Obviously, modifications and alterations will occur to others upon reading and understanding the preceding detailed description. It is intended that the present disclosure be construed as including all such modifications and alterations insofar as they come within the scope of the appended claims or the equivalents thereof.

1. A method of testing for drug susceptibility in a cancer patient, comprising:
obtaining a control test tube (2320) and an assay test tube (2330), each test tube containing blood from the cancer patient;
adding a drug to the assay test tube (2340);
introducing a separator float into the assay test tube (2350);
moving the float into alignment with the cancer cells to capture the cancer cells in an annular volume (2360);
extracting the cancer cells from the annular volume;
visually examining the cancer cells; and
comparing the effect of the drug on cancer cells in the assay test tube to cancer cells in the control test tube (2380).

2. The method of claim 1, wherein the drug is a fluorescently labeled drug.

3. The method of claim 1, wherein a change in the shape of the cancer cells is compared between the assay test tube and the control test tube.

4. The method of claim 1, further comprising staining the cancer cells (2362) prior to visually examining the assay test tube.

5. The method of claim 1, wherein the visual examination is performed by detecting a quantity of fluorescence.

6. The method of claim 1, further comprising visually examining the control test tube (2372).

7. The method of claim 1, wherein the movement of the float is performed by:
centrifuging the assay test tube to move the float into alignment with the cancer cells; and
reducing rotational speed to capture the cancer cells within an annular volume.

8. The method of claim 1, wherein the control test tube and the assay test tube are obtained by receiving a blood sample (2310) from the cancer patient and dividing the blood sample (2315) into the control test tube (2320) and the assay test tube (2330).

9. The method of claim 1, wherein the control test tube and the assay test tube are obtained by receiving two test tubes (2320, 2330), each tube containing the blood of the cancer patient, wherein one test tube is designated as the control test tube and the other test tube is designated as the assay test tube.

10. The method of claim 1, wherein a plurality of assay test tubes are obtained;
wherein the drug is added to each assay test tube, wherein the amount of the drug is different in each assay test tube; and
comparing the effect of the amount of the drug on cancer cells in each assay test tube with cancer cells in the control test tube.

11. The method of claim 1, wherein the drug is added to the assay test tube (2340) after moving the float into alignment with the cancer cells to capture the cancer cells in an annular volume (2360).

12. The method of claim 1, wherein the drug is added to the assay test tube (2340) before moving the float into alignment with the cancer cells to capture the cancer cells in an annular volume (2360)

13. The method of claim 1, wherein the cancer cells are extracted from the annular volume through a sidewall of the assay test tube using a removal device.

14. The method of claim 1, wherein the assay test tube comprises a sidewall and one or more circumferential notches on the assay test tube.

15. The method of claim 1, wherein the separator float (1210) comprises a main body portion (1212), a plurality of axially oriented ridges (1224) protruding from the main body portion, and does not have end sealing ridges (1214).

16. The method of claim 1, wherein the separator float is flexible and has a first cross-sectional diameter when not under centrifugal pressure and a second smaller cross-sectional diameter when under centrifugal pressure.
